(19) United States
(12) Patent Application Publication
(10) Pub. No.: US 2015/0258080 A1
(43) Pub. Date: Sep. 17, 2015

(54) THERAPEUTIC COMBINATIONS WITH ESTROGEN RECEPTOR MODULATORS

(71) Applicant: Genentech, Inc., South San Francisco, CA (US)

(72) Inventors: Jeffrey J. Hager, San Diego, CA (US); Edna Chow Maneval, San Diego, CA (US); Nicholas D. Smith, San Diego, CA (US); Lori Friedman, San Carlos, CA (US); Deepak Sampath, San Francisco, CA (US)

(73) Assignee: Genentech, Inc., South San Francisco, CA (US)

(21) Appl. No.: 14/645,585

(22) Filed: Mar. 12, 2015

Related U.S. Application Data
(60) Provisional application No. 61/952,812, filed on Mar. 13, 2014.

(51) Int. Cl. A61K 31/4439; A61K 31/553; A61K 31/5377; A61K 31/397; A61K 31/416; A61K 31/4245; A61K 31/428; A61K 31/4025; A61K 45/06; A61K 31/517

(52) U.S. Cl. CPC A61K 31/4439; A61K 45/06; A61K 31/553; A61K 31/5377; A61K 31/517; A61K 31/416; A61K 31/4245; A61K 31/428; A61K 31/4025; A61K 31/397

(57) ABSTRACT

Described herein are therapeutic combinations with estrogen receptor modulators for treating diseases or conditions that are mediated or dependent upon estrogen receptors.

### CROSS REFERENCE TO RELATED APPLICATIONS

This non-provisional application filed under 37 CFR §1.53(b), claims the benefit under 35 USC §119(e) of U.S. Provisional Application Ser. No. 61/952,812 filed on 13 Mar. 2014, which is incorporated by reference in entirety.

### FIELD OF THE INVENTION

Described herein are compounds, including pharmaceutically acceptable salts, solvates, metabolites, prodrugs thereof, pharmaceutical compositions comprising such compounds, and methods of using such compounds to treat, prevent or diagnose diseases or conditions that are estrogen sensitive, estrogen receptor dependent or estrogen receptor mediated in combination with other therapeutic agents.

### BACKGROUND OF THE INVENTION

The estrogen receptor (“ER”) is a ligand-activated transcriptional regulatory protein that mediates induction of a variety of biological effects through its interaction with endogenous estrogens. Endogenous estrogens include 17β (beta)-estradiol and estrones. ER has been found to have two isoforms, ER-α (alpha) and ER-β (beta).

Estrogens and estrogen receptors are implicated in a number of diseases or conditions, such as breast cancer, lung cancer, ovarian cancer, colon cancer, prostate cancer, endometrial cancer, uterine cancer, as well as others diseases or conditions.

ARN-810 (GDC-0810, Seragon Pharmaceuticals, Genentech Inc.) is a potent small molecule, nonsteroidal, selective ER modulator that antagonizes the effects of estrogens and induces ER degradation via proteasome. ARN-810 is in clinical trials as an orally-delivered therapy to treat advanced metastatic ER-α positive (ER+) breast cancer.

Non-steroidal, Selective estrogen receptor degraders (SERD) have been described (WO 2011/156518; U.S. Pat. No. 8,703,810; WO 2012/037411; WO 2012/037410; U.S. Pat. No. 8,299,112; U.S. Pat. No. 8,455,534; WO 2013/090829; WO 2013/142266; WO 2014/151899; WO 2013/090836; WO 2014/025138; WO 2014/205136).

Combinations of anti-cancer pharmaceutical therapeutics administered simultaneously or sequentially in a dosing regimen are now common in cancer treatment. Successful combination therapy provides improved and even synergistic effect over mono-therapy, i.e. pharmaceutical treatment limited to one drug (Ouchi et al (2006) Cancer Chemother. Pharmacol. 57:693-702; Higgins et al (2004) Anti-Cancer Drugs 15:503-512). Preclinical research has been the basis for prediction of clinical stage synergy of anti-cancer pharmaceutical therapeutic combinations such as capecitabine and taxanes for the treatment of breast cancer (Sawada et al (1998) Clin. Cancer Res. 4:1013-1019). Certain doses and schedules of combination therapy can improve safety without compromising efficacy (O'Shaughnessy et al (2006) Clin. Breast Cancer April 7(1):42-50). Synergistic effects in vitro have been correlated with clinical stage synergy (Steinbach et al (2003) Clin. Inf. Dis. October 1:37 Suppl 3:S188-224).

### SUMMARY OF THE INVENTION

In one aspect, presented herein are compounds of Formula (A), (B), and (C) that diminish the effects of estrogens with estrogen receptors and/or lower the concentrations of estrogen receptors, and therefore, are useful as agents for the treatment or prevention of diseases or conditions in which the actions of estrogens and/or estrogen receptors are involved in the etiology or pathology of the disease or condition or contribute to at least one symptom of the disease or condition and wherein such actions of estrogens and/or estrogen receptors are undesirable. In some embodiments, compounds disclosed herein are estrogen receptor degrader compounds.

In one aspect, compounds of Formula (A), (B), and (C) are useful for the treatment of ER-related diseases or conditions including, but not limited to, ER-α dysfunction associated with cancer (bone cancer, breast cancer, lung cancer, colorectal cancer, endometrial cancer, prostate cancer, ovarian and uterine cancer), central nervous system (CNS) defects (alcoholism, migraine), cardiovascular system defects (aortic aneurysm, susceptibility to myocardial infarction, aortic valve sclerosis, cardiovascular disease, coronary artery disease, hypertension), hematological system defects (deep vein thrombosis), immune and inflammation diseases (Graves' Disease, arthritis, multiple sclerosis, cirrhosis), susceptibility to infection (hepatitis B, chronic liver disease), metabolic defects (bone density, cholestasis, hypospadias, obesity, osteoarthritis, osteopenia, osteoporosis), neurological defects (Alzheimer's disease, Parkinson's disease, migraine, vertigo), psychiatric defects (anorexia nervosa, attention deficit hyperactivity disorder (ADHD), dementia, major depressive disorder, psychosis), uterine diseases (e.g. leiomyoma, uterine leiomyoma, endometrial hyperplasia, endometriosis), and reproductive defects (age of menarche, endometriosis, infertility).

In one aspect, described herein are compounds of Formula (A), (B), and (C), pharmaceutically acceptable salts, solvates, metabolites and prodrugs thereof. Compounds described herein are estrogen receptor modulators. In some embodiments, the compound of Formula (A), (B), or (C) is an estrogen receptor antagonist. In some embodiments, the compound of Formula (A), (B), or (C) is an estrogen receptor degrader. In some embodiments, the compound of Formula (A), (B), or (C) is an estrogen receptor antagonist as well as an estrogen receptor degrader. In some embodiments, the compound of Formula (A), (B), or (C) displays minimal or no estrogen receptor agonist activity. In some embodiments, in the context of treating cancers, the compound of Formula (A), (B), or (C) may offer improved therapeutic activity characterized by complete or longer-lasting tumor regression, a lower incidence or rate of development of resistance to treatment, and/or a reduction in tumor invasiveness.

In some embodiments, compounds disclosed herein have high specificity for the estrogen receptor and have desirable, tissue-selective pharmacological activities. Desirable, tissue-selective pharmacological activities include, but are not limited to, ER antagonist activity in breast cells and no ER agonist activity in uterine cells. In some embodiments, compounds disclosed herein are estrogen receptor degraders that display full estrogen receptor antagonist activity with negligible or minimal estrogen receptor agonist activity.

In some embodiments, compounds disclosed herein are estrogen receptor degraders. In some embodiments, compounds disclosed herein are estrogen receptor antagonists. In some embodiments, compounds disclosed herein have minimal or negligible estrogen receptor agonist activity.

In some embodiments, presented herein are compounds selected from active metabolites, tautomers, pharmaceutically acceptable solvates, pharmaceutically acceptable salts or prodrugs of a compound of Formula (A), (B), or (C).

In one aspect, described herein is a method for treating an ER-related disease or condition in a patient comprising administering to the patient an estrogen receptor modulator compound of Formula (A) in combination with a second therapeutic agent, wherein the compound of Formula (A) has the structure:

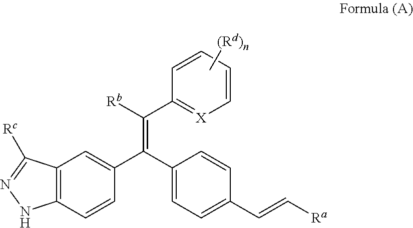

wherein,

R^{a} is —CO₂H or a 5-membered heterocycle selected from the group consisting of

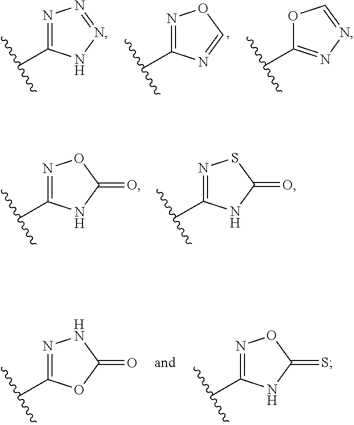

R^{b} is C₁-C₆alkyl or C₃-C₆cycloalkyl;

R^{c} is H or F;

each R^{d} is independently selected from H, halogen, —CN, —OR^{e}, —NHR^{e}, —NR^{e}R^{f}, —SR^{e}, —S(═O)R^{f}, —S(═O)₂R^{f}, C₁-C₆alkyl, and C₁-C₆fluoroalkyl;

each R^{e} is independently selected from H, —C(═O)R^{f}, —C(═O)OR^{f}, —C(═O)NHR^{f}, C₁-C₆alkyl, C₁-C₆fluoroalkyl, C₃-C₆cycloalkyl, substituted or unsubstituted monocyclic C₂-C₆heterocycloalkyl, substituted or unsubstituted phenyl, and substituted or unsubstituted monocyclic heteroaryl;

each R^{f} is independently selected from C₁-C₆alkyl, C₁-C₆fluoroalkyl, C₃-C₆cycloalkyl, substituted or unsubstituted monocyclic C₂-C₆heterocycloalkyl, substituted or unsubstituted phenyl, and substituted or unsubstituted monocyclic heteroaryl;

X is CH or N; and

n is 0, 1, or 2, or a pharmaceutically acceptable salt, or solvate thereof.

In some embodiments, the estrogen receptor modulator is a compound of Formula (A-1) having the structure:

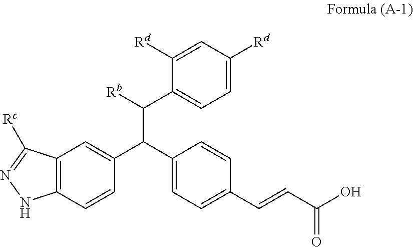

or a pharmaceutically acceptable salt, or solvate thereof.

In some embodiments, the estrogen receptor modulator is compound 1-1, 1-2, 1-3, 1-4, 1-5, 1-6, 1-7, 1-8, 1-9, 1-10, 1-11, or 1-12, or a pharmaceutically acceptable salt, or solvate thereof.

In some embodiments, the estrogen receptor modulator is compound 1-3, also known as ARN-810 (GDC-0810).

In some embodiments, the ER-related disease or condition is cancer. In some embodiments, the cancer is a breast cancer. In some embodiments, the breast cancer is a metastatic breast cancer. In some embodiments, the breast cancer is a hormone resistant breast cancer. In some embodiments, the breast cancer is an estrogen receptor positive breast cancer. In some embodiments, the breast cancer is a HER2 positive breast cancer. In some embodiments, the breast cancer is a HER2 negative breast cancer. In some embodiments, the breast cancer is resistant to treatment with an aromatase inhibitor. In some embodiments, the aromatase inhibitor is anastrozole, letrozole, or exemestane. In some embodiments, the patient has a tumor. In some embodiments, the patient is a female patient and is pre-menopausal or post-menopausal. In some embodiments, the patient has failed one or more anti-cancer therapies. In some embodiments, the patient has received a chemotherapeutic agent, a biological therapy, a cancer vaccine, an angiogenesis inhibitor, hormone therapy, radiation therapy, surgery, or any combination thereof. In some embodiments, the biological therapy is a peptide, a cytokine, an antibody, a therapeutic virus, a therapeutic bacterium, gene therapy, siRNA, adoptive T-cell transfer, or any combination thereof. In some embodiments, the patient has received an aromatase inhibitor, a selective estrogen receptor modulator (SERM), a selective estrogen degrader (SERD), a phosphoinositide 3-kinase (PI3K)/mTOR pathway inhibitor such as GDC-0032 and GDC-0941, a CDK 4/6 inhibitor such as palbociclib, a HER-2 inhibitor, an EGFR inhibitor, a PD-1 inhibitor, poly ADP-ribose polymerase (PARP) inhibitor, a histone deacetylase (HDAC) inhibitor, an HSP90 inhibitor, or any combination thereof. In some embodiments, the patient has received fulvestrant, tamoxifen, anastrozole, letrozole, exemestane, goserelin, leuprolide, raloxifene, toremifene, megestrol acetate, bazedoxifene, or any combination thereof. In some embodiments, the patient has received an anthracycline, a taxane, a platinum agent, an epothilone, or a nucleoside analog. In some embodiments, the patient has received cisplatin, carboplatin, capecitabine, cyclophosphamide, docetaxel, doxorubicin, epirubicin, eribulin, fluorouracil, gemcitabine, ixabepilone, mitoxantrone, methotrexate, paclitaxel, pamidronate, vinorelbine, or any combination thereof. In some embodiments, the patient has received pertuzumab, trastuzumab, lapatinib, everolimus, bevacizumab, or temsirolimus, or any combination thereof. In some embodiments, the second therapeutic agent is an anti-cancer agent. In some embodiments, the anti-cancer agent is doxorubicin, cyclophosphamide, capecitabine, vinorelbine, paclitaxel, doxetaxel, or cisplatin. In some embodiments, the second therapeutic agent is an aromatase inhibitor, a phosphoinositide 3-kinase (PI3K)/mTOR pathway inhibitor, a CDK 4/6 inhibitor, a HER-2 inhibitor, an EGFR inhibitor, a PD-1 inhibitor, poly ADP-ribose polymerase (PARP) inhibitor, a histone deacetylase (HDAC) inhibitor, an HSP90 inhibitor, a VEGFR inhibitor, an AKT inhibitor, chemotherapy, or any combination thereof.

In one aspect, described herein is a method for treating an ER-related disease or condition in a patient comprising administering to the patient an estrogen receptor modulator compound of Formula (B) in combination with a second therapeutic agent, wherein the compound of Formula (B) has the structure:

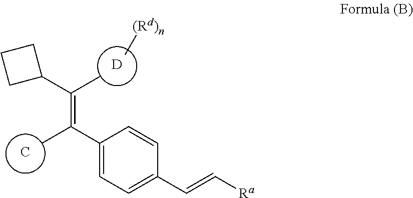

wherein,

R^{a} is —CO₂H or a 5-membered heterocycle selected from the group consisting of

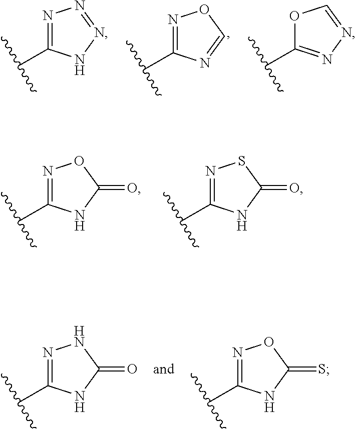

ring C is

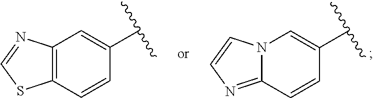 [SMILES: *c1ccc2scnc2c1 ; *c1ccc2nccn2c1]

ring D is phenyl or thienyl;

each R^{d} is independently selected from H, halogen, —CN, —OR^{e}, —NHR^{e}, —NR^{e}R^{f}, —SR^{e}, —S(═O)R^{f}, —S(═O)₂R^{f}, C₁-C₆ alkyl, and C₁-C₆fluoroalkyl;

each R^{e} is independently selected from H, —C(═O)R^{f}, —C(═O)OR^{f}, —C(═O)NHR^{f}, C₁-C₆ alkyl, C₁-C₆fluoroalkyl, C₃-C₆cycloalkyl, substituted or unsubstituted monocyclic C₂-C₆ heterocycloalkyl, substituted or unsubstituted phenyl, and substituted or unsubstituted monocyclic heteroaryl;

each R^{f} is independently selected from C₁-C₆ alkyl, C₁-C₆fluoroalkyl, C₃-C₆ cycloalkyl, substituted or unsubstituted monocyclic C₂-C₆ heterocycloalkyl, substituted or unsubstituted phenyl, and substituted or unsubstituted monocyclic heteroaryl;

n is 0, 1, or 2; or a pharmaceutically acceptable salt, or solvate thereof.

In some embodiments, the estrogen receptor modulator is compound 2-1, 2-2, 2-3, 2-4, or 2-5, or a pharmaceutically acceptable salt, or solvate thereof.

In some embodiments, the ER-related disease or condition is cancer. In some embodiments, the cancer is a breast cancer. In some embodiments, the breast cancer is a metastatic breast cancer. In some embodiments, the breast cancer is a hormone resistant breast cancer. In some embodiments, the breast cancer is an estrogen receptor positive breast cancer. In some embodiments, the breast cancer is a HER2 positive breast cancer. In some embodiments, the breast cancer is a HER2 negative breast cancer. In some embodiments, the breast cancer is resistant to treatment with an aromatase inhibitor. In some embodiments, the aromatase inhibitor is anastrozole, letrozole, or exemestane. In some embodiments, the patient has a tumor. In some embodiments, the patient is a female patient and is pre-menopausal or post-menopausal. In some embodiments, the patient has failed one or more anti-cancer therapies. In some embodiments, the patient has received a chemotherapeutic agent, a biological therapy, a cancer vaccine, an angiogenesis inhibitor, hormone therapy, radiation therapy, surgery, or any combination thereof. In some embodiments, the biological therapy is a peptide, a cytokine, an antibody, a therapeutic virus, a therapeutic bacterium, gene therapy, siRNA, adoptive T-cell transfer, or any combination thereof. In some embodiments, the patient has received an aromatase inhibitor, a selective estrogen receptor modulator (SERM), a selective estrogen degrader (SERD), a phosphoinositide 3-kinase (PI3K)/mTOR pathway inhibitor, a CDK 4/6 inhibitor, a HER-2 inhibitor, an EGFR inhibitor, a PD-1 inhibitor, poly ADP-ribose polymerase (PARP) inhibitor, a histone deacetylase (HDAC) inhibitor, an HSP90 inhibitor, or any combination thereof. In some embodiments, the patient has received fulvestrant, tamoxifen, anastrozole, letrozole, exemestane, goserelin, leuprolide, raloxifene, toremifene, megestrol acetate, bazedoxifene, or any combination thereof. In some embodiments, the patient has received an anthracycicine, a taxane, a platinum agent, an epothilone, or a nucleoside analog. In some embodiments, the patient has received cisplatin, carboplatin, capecitabine, cyclophosphamide, docetaxel, doxorubicin, epirubicin, eribulin, fluorouracil, gemcitabine, ixabepilone, mitoxantrone, methotrexate, paclitaxel, pamidronate, vinorelbine, or any combination thereof. In some embodiments, the patient has received pertuzumab, trastuzumab, lapatinib, everolimus, bevacizumab, or temsirolimus, or any combination thereof. In some embodiments, the second therapeutic agent is an anti-cancer agent. In some embodiments, the anti-cancer agent is doxorubicin, cyclophosphamide, capecitabine, vinorelbine, paclitaxel, doxetaxel, or cisplatin. In some embodiments, the second therapeutic agent is an aromatase inhibitor, a phosphoinositide 3-kinase (PI3K)/mTOR pathway inhibitor, a CDK 4/6 inhibitor, a HER-2 inhibitor, an EGFR inhibitor, a PD-1 inhibitor, poly ADP-ribose polymerase (PARP) inhibitor, a histone deacetylase (HDAC) inhibitor, an HSP90 inhibitor, a VEGFR inhibitor, an AKT inhibitor, chemotherapy, or any combination thereof.

In one aspect, described herein is a method for treating an ER-related disease or condition in a patient comprising administering to the patient an estrogen receptor modulator compound of Formula (C) in combination with a second therapeutic agent, wherein the compound of Formula (C) has the structure:

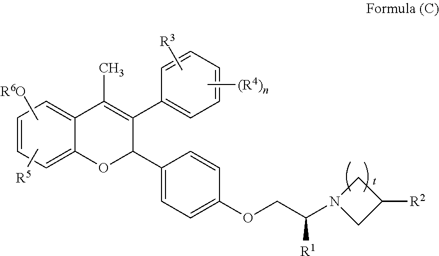

wherein, R¹ is H, C₁-C₄alkyl, or C₁-C₄fluoroalkyl;

R² is H, F, C₁-C₄alkyl or C₁-C₄fluoroalkyl;

R³ is H, halogen, —CN, —OR⁶, —NHR⁶, —NR⁶R⁷, —SR⁶, —S(═O)R⁷, —S(═O)₂R⁷, C₁-C₄alkyl, or C₁-C₄fluoroalkyl;

each R⁴ is independently selected from H, halogen, —CN, —OH, C₁-C₆alkyl, C₁-C₄fluoroalkyl, C₁-C₄fluoroalkoxy, and C₁-C₄alkoxy;

each R⁵ is H, F, Cl, —OH, —CH₃, —CF₃, or —OCH₃;

each R⁶ is independently selected from H, —C(═O)R⁷, —C(═O)OR⁷, —C(═O)NHR⁷, C₁-C₆alkyl, C₁-C₆fluoroalkyl, C₃-C₆cycloalkyl, substituted or unsubstituted C₂-C₆heterocycloalkyl, substituted or unsubstituted phenyl, and substituted or unsubstituted monocyclic heteroaryl; each R⁷ is independently selected from C₁-C₆alkyl, C₁-C₆fluoroalkyl, substituted or unsubstituted C₃-C₆cycloalkyl, substituted or unsubstituted monocyclic C₂-C₆heterocycloalkyl, substituted or unsubstituted phenyl, and substituted or unsubstituted monocyclic heteroaryl;

n is 0, 1, or 2;

t is 1 or 2;

or a pharmaceutically acceptable salt, or solvate thereof.

In some embodiments, the estrogen receptor modulator of Formula (C) is selected from Formulas (C-1), (C-2), (C-3), (C-4), (C-5), and (C-6), having the structures:

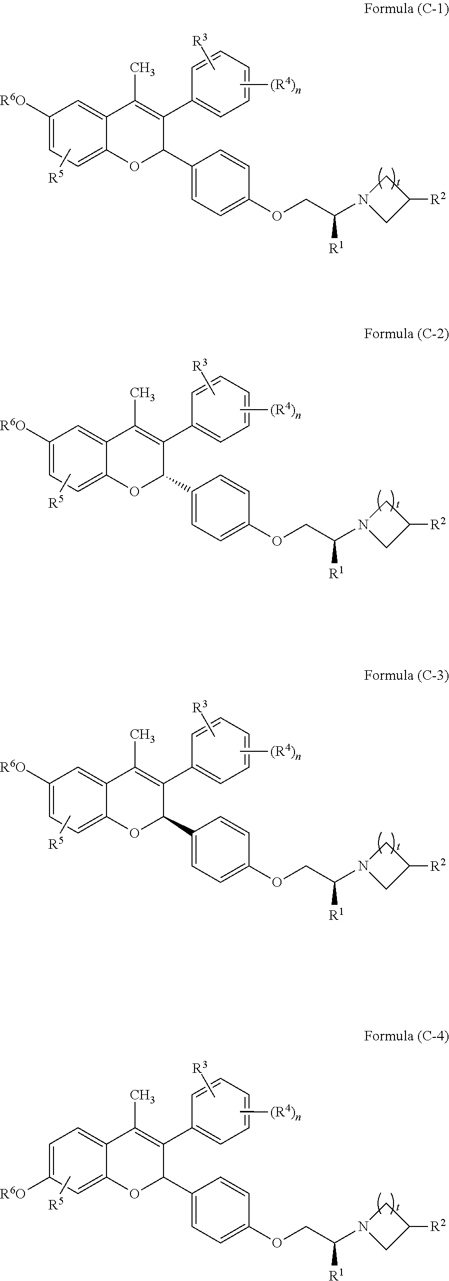

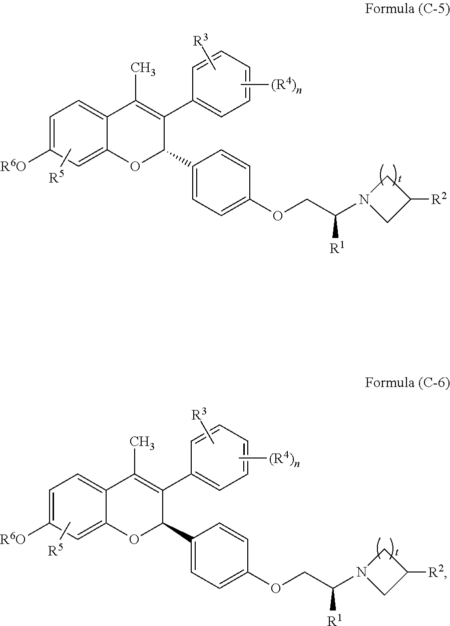

or a pharmaceutically acceptable salt, or solvate thereof.

In some embodiments, the estrogen receptor modulator is compound 4-1, 4-2, 4-3, 4-4, 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 4-25, 4-26, 4-27, 4-28, 4-29, 4-30, 4-31, 4-32, 4-33; 4-34, 4-35, or a pharmaceutically acceptable salt, or solvate thereof.

In some embodiments, the ER-related disease or condition is cancer. In some embodiments, the cancer is a breast cancer. In some embodiments, the breast cancer is a metastatic breast cancer. In some embodiments, the breast cancer is a hormone resistant breast cancer. In some embodiments, the breast cancer is an estrogen receptor positive breast cancer. In some embodiments, the breast cancer is a HER2 positive breast cancer. In some embodiments, the breast cancer is a HER2 negative breast cancer. In some embodiments, the breast cancer is resistant to treatment with an aromatase inhibitor. In some embodiments, the aromatase inhibitor is anastrozole, letrozole, or exemestane. In some embodiments, the patient has a tumor. In some embodiments, the patient is a female patient and is pre-menopausal or post-menopausal. In some embodiments, the patient has failed one or more anti-cancer therapies. In some embodiments, the patient has received a chemotherapeutic agent, a biological therapy, a cancer vaccine, an angiogenesis inhibitor, hormone therapy, radiation therapy, surgery, or any combination thereof. In some embodiments, the biological therapy is a peptide, a cytokine, an antibody, a therapeutic virus, a therapeutic bacterium, gene therapy, siRNA, adoptive T-cell transfer, or any combination thereof. In some embodiments, the patient has received an aromatase inhibitor, a selective estrogen receptor modulator (SERM), a selective estrogen degrader (SERD), a phosphoinositide 3-kinase (PI3K)/mTOR pathway inhibitor, a CDK 4/6 inhibitor, a HER-2 inhibitor, an EGFR inhibitor, a PD-1 inhibitor, poly ADP-ribose polymerase (PARP) inhibitor, a histone deacetylase (HDAC) inhibitor, an HSP90 inhibitor, or any combination thereof. In some embodiments, the patient has received fulvestrant, tamoxifen, anastrozole, letrozole, exemestane, goserelin, leuprolide, raloxifene, toremifene, megestrol acetate, bazedoxifene, or any combination thereof. In some embodiments, the patient has received an anthracycicine, a taxane, a platinum agent, an epothilone, or a nucleoside analog. In some embodiments, the patient has received cisplatin, carboplatin, capecitabine, cyclophosphamide, docetaxel, doxorubicin, epirubicin, eribulin, fluorouracil, gemcitabine, ixabepilone, mitoxantrone, methotrexate, paclitaxel, pamidronate, vinorelbine, or any combination thereof. In some embodiments, the patient has received pertuzumab, trastuzumab, lapatinib, everolimus, bevacizumab, or temsirolimus, or any combination thereof. In some embodiments, the second therapeutic agent is an anti-cancer agent. In some embodiments, the anti-cancer agent is doxorubicin, cyclophosphamide, capecitabine, vinorelbine, paclitaxel, docetaxel, or cisplatin. In some embodiments, the second therapeutic agent is an aromatase inhibitor, a phosphoinositide 3-kinase (PI3K)/mTOR pathway inhibitor, a CDK 4/6 inhibitor, a HER-2 inhibitor, an EGFR inhibitor, a PD-1 inhibitor, poly ADP-ribose polymerase (PARP) inhibitor, a histone deacetylase (HDAC) inhibitor, an HSP90 inhibitor, a VEGFR inhibitor, an AKT inhibitor, chemotherapy, or any combination thereof.

In some embodiments, the compound of Formula (A), (B) or (C), or a pharmaceutically acceptable salt, or solvate thereof is administered as a pharmaceutical composition, wherein the pharmaceutical composition is formulated for intravenous injection, subcutaneous injection, oral administration, or topical administration. In some embodiments, the pharmaceutical composition is a tablet, a pill, a capsule, a liquid, a suspension, a gel, a dispersion, a solution, an emulsion, an ointment, or a lotion.

In some embodiments, the pharmaceutical composition described herein further comprises, in addition to the compound of Formula (A), (B), or (C), one or more additional therapeutically active agents. In some embodiments, one or more additional therapeutically active agents are selected from: corticosteroids, anti-emetic agents, analgesics, anti-cancer agents, anti-inflammatories, kinase inhibitors, antibodies, HSP90 inhibitors, histone deacetylase (HDAC) inhibitors, poly ADP-ribose polymerase (PARP) inhibitors, and aromatase inhibitors.

Embodiments include a method comprising administering a compound of Formula (A), (B), or (C), or a pharmaceutically acceptable salt or prodrug thereof, to a human with a diseases or condition that is estrogen sensitive, estrogen receptor meditated or estrogen receptor dependent. In some embodiments, the human is already being administered one or more additional therapeutically active agents other than a compound of Formula (A), (B), or (C), or a pharmaceutically acceptable salt or prodrug thereof. In some embodiments, the method further comprises administering one or more additional therapeutically active agents other than a compound of Formula (A), (B), or (C), or a pharmaceutically acceptable salt or prodrug thereof.

In some embodiments, the one or more additional therapeutically active agents other than a compound of Formula (A), (B), or (C), or a pharmaceutically acceptable salt or prodrug thereof, are selected from: corticosteroids, anti-emetic agents, analgesics, anti-cancer agents, anti-inflammatories, kinase inhibitors, antibodies, HSP90 inhibitors, histone deacetylase (HDAC) inhibitors, and aromatase inhibitors.

Pharmaceutical formulations described herein are administered to a mammal in a variety of ways, including but not limited to, oral, parenteral (e.g., intravenous, subcutaneous, intramuscular), buccal, topical or transdermal administration routes. The pharmaceutical formulations described herein include, but are not limited to, aqueous liquid dispersions, self-emulsifying dispersions, solid solutions, liposomal dispersions, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compound of Formula (A), (B), or (C), or a pharmaceutically acceptable salt or prodrug thereof, is administered orally, systemically, intravenously, subcutaneously or topically. In such embodiments, the compound of Formula (A), (B), or (C), or a pharmaceutically acceptable salt or prodrug thereof, is formulated into a variety of topically administrable compositions, such as solutions, suspensions, lotions, gels, pastes, shampoos, scrubs, rubs, smears, medicated sticks, medicated bandages, balms, creams or ointments. In some embodiments, the compound of Formula (A), (B), or (C), or a pharmaceutically acceptable salt or prodrug thereof, is administered topically to the skin of mammal.

In another aspect is the use of a compound of Formula (A), (B), or (C), or a pharmaceutically acceptable salt or prodrug thereof, in the manufacture of a medicament for treating a disease, disorder or conditions in which the activity of estrogen receptors contributes to the pathology and/or symptoms of the disease or condition. In one aspect, the disease or condition is any of the diseases or conditions specified herein.

In any of the aforementioned aspects are further embodiments in which the effective amount of the compound of Formula (A), (B), or (C), or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) adminstered non-systemically or locally to the mammal.

In any of the aforementioned aspects are further embodiments in which the effective amount of the additional therapeutic agent (other than the compound of Formula (A), (B), or (C), or a pharmaceutically acceptable salt thereof), is: (a) systemically administered to the mammal; and/or (b) administered orally to the mammal; and/or (c) intravenously administered to the mammal; and/or (d) administered by injection to the mammal; and/or (e) administered topically to the mammal; and/or (f) adminstered non-systemically or locally to the mammal.

In any of the aforementioned aspects are further embodiments comprising single administrations of the effective amount of the compound, including further embodiments in which (i) the compound is administered once; (ii) the compound is administered to the mammal multiple times over the span of one day; (iii) continually; or (iv) continuously.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

Also provided is a method of reducing ER activation in a mammal comprising administering to the mammal at least one compound having the structure of Formula (A), (B), or (C), or a pharmaceutically acceptable salt thereof. In some embodiments, the method comprises reducing ER activation in breast cells, lung cells, ovarian cells, colon cells, prostate cells, endometrial cells, or uterine cells in the mammal. In some embodiments, the method comprises reducing ER activation in breast cells, ovarian cells, colon cells, prostate cells, endometrial cells, or uterine cells in the mammal. In some embodiments, the method of reducing ER activation in the mammal comprises reducing the binding of estrogens to estrogen receptors in the mammal. In some embodiments, the method of reducing ER activation in the mammal comprises reducing ER concentrations in the mammal.

In one aspect is the use of a compound of Formula (A), (B), or (C), or a pharmaceutically acceptable salt thereof, in the treatment or prevention of diseases or conditions of the uterus in a mammal. In some embodiments, the disease or condition of the uterus is leiomyoma, uterine leiomyoma, endometrial hyperplasia, or endometriosis. In some embodiments, the disease or condition of the uterus is a cancerous disease or condition of the uterus. In some other embodiments, the disease or condition of the uterus is a non-cancerous disease or condition of the uterus.

In one aspect is the use of a compound of Formula (A), (B), or (C), or a pharmaceutically acceptable salt thereof, in the manufacture of a medicament for the treatment of diseases or conditions that are estrogen sensitive, estrogen receptor dependent or estrogen receptor mediated. In some embodiments, the disease or condition is breast cancer, lung cancer, ovarian cancer, colon cancer, prostate cancer, endometrial cancer, or uterine cancer. In some embodiments, the disease or condition is described herein.

In some cases disclosed herein is the use of a compound of Formula (A), (B), or (C), or a pharmaceutically acceptable salt thereof, in the treatment or prevention of diseases or conditions that are estrogen sensitive, estrogen receptor dependent or estrogen receptor mediated. In some embodiments, the disease or condition is described herein.

In any of the embodiments disclosed herein, the mammal is a human.

In some embodiments, compounds provided herein are used to diminish, reduce, or eliminate the activity of estrogen receptors.

Articles of manufacture, which include: packaging material; a compound of Formula (A), (B), or (C), or a pharmaceutically acceptable salt, active metabolite, prodrug, or pharmaceutically acceptable solvate thereof, or composition thereof, within the packaging material; and a label that indicates that the compound or pharmaceutically acceptable salt, active metabolite, prodrug, or pharmaceutically acceptable solvate thereof, or composition thereof, or composition thereof, is used for reducing, diminishing or eliminating the effects of estrogen receptors, or for the treatment, prevention or amelioration of one or more symptoms of a disease or condition that would benefit from a reduction or elimination of estrogen receptor activity, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description

### BRIEF DESCRIPTION OF THE DRAWINGS

FIG. 1 shows the fitted tumor volume change over 42 days in cohorts of 8 immunocompromised mice bearing HCI-003 breast tumor (BC PDX model) xenografts harboring ESR1 WT (wild type) and PIK3CA H1047R (PI3Kα) mutation, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 1-3 from Table 1 at 10 mg/kg, and the combination of ERM 1-3 and GDC-0032. Vehicle (+) is solvent/buffer with ethynyl estradiol (0.1 mg/kg). Vehicle (−) is solvent/buffer without ethynyl estradiol.

FIG. 2 shows the fitted tumor volume change over 38 days in cohorts of 8 immunocompromised mice bearing HCI-005 breast tumor (BC PDX model) xenografts harboring ESR1 L536P mutant, HER2+, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 1-3 from Table 1 at 100 mg/kg, and the combination of ERM 1-3 and GDC-0032.

FIG. 3 shows the fitted tumor volume change over 27 days in cohorts of 8 immunocompromised mice bearing HCI-011 breast tumor (BC PDX model) xenografts harboring ESR1 WT (wild type) and PIK3CA E545K (PI3Kα) mutation, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 1-3 from Table 1 at 10 mg/kg, and the combination of ERM 1-3 and GDC-0032.

FIG. 4 shows the fitted tumor volume change over 26 days in cohorts of 8 or 9 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, PI3K inhibitor GDC-0032 at 2 mg/kg and 5 mg/kg, estrogen receptor modulator (ERM) 1-3 from Table 1 at 30 mg/kg and 100 mg/kg, and combinations of ERM 1-3 (30 mg/kg) and GDC-0032 (2 and 5 mg/kg).

FIG. 5 shows the fitted tumor volume change over 42 days in cohorts of 7 immunocompromised mice bearing HCI-003 breast tumor (BC PDX model) xenografts harboring ESR1 WT (wild type) and PIK3CA H1047R (PI3Kα) mutation, dosed daily by 100 microliter (μl) PO (oral) administration with Vehicle (+), Vehicle (−), pan-PI3K inhibitor GDC-0941 at 50, 100 and 150 mg/kg, estrogen receptor modulator (ERM) 1-3 from Table 1 at 100 mg/kg, and the combination of ERM 1-3 and GDC-0941.

FIG. 6 shows the fitted tumor volume change over 40 days in cohorts of 8 immunocompromised mice bearing HCI-005 breast tumor (BC PDX model) xenografts harboring ESR1 L536P mutant, and HER2+, dosed daily by 100 microliter (μl) PO (oral) administration with Vehicle (+), Vehicle (−), pan-PI3K inhibitor GDC-0941 at 50, 100 and 150 mg/kg, estrogen receptor modulator (ERM) 1-3 from Table 1 at 100 mg/kg, and the combination of ERM 1-3 and GDC-0941.

FIG. 7 shows the fitted tumor volume change over 27 days in cohorts of 8 immunocompromised mice bearing HCI-011 breast tumor (BC PDX model) xenografts harboring ESR1 WT (wild type) and PIK3CA E545K (PI3Kα) mutation, dosed daily by 100 microliter (μl) PO (oral) administration with Vehicle (+), Vehicle (−), pan-PI3K inhibitor GDC-0941 at 50, 100 and 150 mg/kg, estrogen receptor modulator (ERM) 1-3 from Table 1 at 100 mg/kg, the combination of ERM 1-3 and GDC-0941, and ERM 4-35 from Table 3.

FIG. 8 shows the fitted tumor volume change over 25 days in cohorts of 8 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, pan-PI3K inhibitor GDC-0941 at 50, 100 and 150 mg/kg, estrogen receptor modulator (ERM) 1-3 from Table 1 at 100 mg/kg, and the combination of ERM 1-3 (30 mg/kg) and GDC-0941.

FIG. 9 shows the fitted tumor volume change over 41 days in cohorts of 7 immunocompromised mice bearing HCI-003 breast tumor (BC PDX model) xenografts harboring ESR1 WT (wild type) and PIK3CA H1047R (PI3Kα) mutation, dosed daily by 100 microliter (μl) PO (oral) administration with Vehicle (+), Vehicle (−), AKT inhibitor GDC-0068 at 20 and 40 mg/kg, estrogen receptor modulator (ERM) 1-3 from Table 1 at 100 mg/kg, the combination of ERM 1-3 and GDC-0068, and ERM 4-35 from Table 3.

FIG. 10 shows the fitted tumor volume change over 23 days in cohorts of 10 immunocompromised mice bearing HCI-011 breast tumor (BC PDX model) xenografts harboring ESR1 WT (wild type) and PIK3CA E545K (PI3Kα) mutation, dosed daily by 100 microliter (μl) PO (oral) administration with Vehicle (+), Vehicle (−), AKT inhibitor GDC-0068 at 20 and 40 mg/kg, estrogen receptor modulator (ERM) 1-3 from Table 1 at 100 mg/kg, the combination of ERM 1-3 and GDC-0941, estrogen receptor modulator (ERM) 4-34 from Table 3 at 100 mg/kg, and the combination of ERM 4-34 and GDC-0068.

FIG. 11 shows the fitted tumor volume change over 26 days in cohorts of 9 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, AKT inhibitor GDC-0068 at 20 and 40 mg/kg, estrogen receptor modulator (ERM) 1-3 from Table 1 at 100 mg/kg, and the combination of ERM 1-3 (30 mg/kg) and GDC-0068.

FIG. 12 shows the fitted tumor volume change over 35 days in cohorts of 8 immunocompromised mice bearing HCI-003 breast tumor (BC PDX model) xenografts harboring ESR1 WT (wild type) and PIK3CA H1047R (PI3Kα) mutation, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 4-34 from Table 3 at 10 mg/kg, and the combination of ERM 4-34 and GDC-0032.

FIG. 13 shows the fitted tumor volume change over 38 days in cohorts of 8 immunocompromised mice bearing HCI-005 breast tumor (BC PDX model) xenografts harboring ESR1 L536P mutant, HER2+, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 4-34 from Table 3 at 100 mg/kg, and the combination of ERM 4-34 and GDC-0032.

FIG. 14 shows the fitted tumor volume change over 25 days in cohorts of 8 immunocompromised mice bearing HCI-011 breast tumor (BC PDX model) xenografts harboring ESR1 WT (wild type) and PIK3CA E545K (PI3Kα) mutation, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 4-34 from Table 3 at 100 mg/kg, and the combination of ERM 4-34 and GDC-0032.

FIG. 15 shows the fitted tumor volume change over 26 days in cohorts of 8 or 9 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, PI3K inhibitor GDC-0032 at 2 mg/kg and 5 mg/kg, estrogen receptor modulator (ERM) 4-34 from Table 3 at 10 mg/kg and 100 mg/kg, and the combination of ERM 4-34 (10 mg/kg) and GDC-0032 (2 mg/kg).

FIG. 16 shows the fitted tumor volume change over 42 days in cohorts of 7 immunocompromised mice bearing HCI-005 breast tumor (BC PDX model) xenografts harboring ESR1 L536P mutant, and HER2+, dosed daily by 100 microliter (μl) PO (oral) administration with Vehicle (+), Vehicle (−), pan-PI3K inhibitor GDC-0941 at 100 and 150 mg/kg, estrogen receptor modulator (ERM) 4-34 from Table 3 at 100 mg/kg, and the combination of ERM 4-34 (100 mg/kg) and GDC-0941 (100 mg/kg).

FIG. 17 shows the fitted tumor volume change over 25 days in cohorts of 8 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, pan-PI3K inhibitor GDC-0941 at 50, 100 and 150 mg/kg, estrogen receptor modulator (ERM) 4-34 from Table 3 at 100 mg/kg, and the combination of ERM 1-3 (100 mg/kg) and GDC-0941 (100 mg/kg).

FIG. 18 shows the fitted tumor volume change over 26 days in cohorts of 9 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, AKT inhibitor GDC-0068 at 20 and 40 mg/kg, estrogen receptor modulator (ERM) 4-34 from Table 3 at 100 mg/kg, and the combination of ERM 4-34 (100 mg/kg) and GDC-0068 (40 mg/kg).

### DETAILED DESCRIPTION OF EXEMPLARY EMBODIMENTS

Estrogen receptor alpha (ER-α; NR3A1) and estrogen receptor beta (ER-β; NR3A2) are steroid hormone receptors, which are members of the large nuclear receptor superfamily. Nuclear receptors share a common modular structure, which minimally includes a DNA binding domain (DBD) and a ligand binding domain (LBD). Steroid hormone receptors are soluble, intracellular proteins that act as ligand-regulated transcription factors. Vertebrates contain five closely related steroid hormone receptors (estrogen receptor, androgen receptor, progesterone receptor, glucocorticoid receptor, mineralcorticoid receptor), which regulate a wide spectrum of reproductive, metabolic and developmental activities. The activities of ER are controlled by the binding of endogenous estrogens, including 1713-estradiol and estrones.

The ER-α gene is located on 6q25.1 and encodes a 595 AA protein. The ER-β gene resides on chromosome 14q23.3 and produces a 530 AA protein. However, due to alternative splicing and translation start sites, each of these genes can give rise to multiple isoforms. In addition to the DNA binding domain (called C domain) and ligand binding domain (E domain) these receptors contain an N-terminal (A/B) domain, a hinge (D) domain that links the C and E domains, and a C-terminal extension (F domain) (Gronemeyer and Laudet; Protein Profile 2: 1173-1308, 1995). While the C and E domains of ER-α and ER-β are quite conserved (95% and 55% amino acid identity, respectively), conservation of the A/B, D and F domains is poor (below 30% amino acid identity). Both receptors are involved in the regulation and development of the female reproductive tract but also play various roles in the central nervous system, cardiovascular systems and bone metabolism.

The ligand binding pocket of steroid hormone receptors is deeply buried within the ligand binding domain. Upon binding, the ligand becomes part of the hydrophobic core of this domain. Consequently most steroid hormone receptors are instable in the absence of hormone and require assistance from chaperones, such as Hsp90, in order to maintain hormone-binding competency. The interaction with Hsp90 also controls nuclear translocation of these receptors. Ligand-binding stabilizes the receptor and initiates sequential conformational changes that release the chaperones, alter the interactions between the various receptor domains and remodel protein interaction surfaces that allow these receptors to translocate into the nucleus, bind DNA and engage in interactions with chromatin remodeling complexes and the transcriptional machinery. Although ER can interact with Hsp90, this interaction is not required for hormone binding and, dependent on the cellular context, apo-ER can be both cytoplasmic and nuclear. Biophysical studies indicated that DNA binding rather than ligand binding contributes to the stability of the receptor (Greenfield et al., Biochemistry 40: 6646-6652, 2001).

ER can interact with DNA either directly by binding to a specific DNA sequence motif called estrogen response element (ERE) (classical pathway), or indirectly via protein-protein interactions (nonclassical pathway) (Welboren et al., Endocrine-Related Cancer 16: 1073-1089, 2009). In the nonclassical pathway, ER has been shown to tether to other transcription factors including SP-1, AP-1 and NF-κB. These interactions appear to play critical roles in the ability of ER to regulate cell proliferation and differentiation.

Both types of ER DNA interactions can result in gene activation or repression dependent on the transcriptional coregulators that are recruited by the respective ER-ERE complex (Klinge, Steroid 65: 227-251, 2000). The recruitment of coregulators is primarily mediated by two protein interaction surfaces, the AF2 and AF1. AF2 is located in the ER E-domain and its conformation is directly regulated by the ligand (Brzozowski et al., Nature 389: 753-758, 1997). Full agonists appear to promote the recruitment of co-activators, whereas weak agonists and antagonists facilitate the binding of co-repressors. The regulation of protein with the AF1 is less well understood but can be controlled by serine phosphorylation (Ward and Weigel, Biofactors 35: 528-536, 2009). One of the involved phosphorylation sites (S118) appears to control the transcriptional activity of ER in the presence of antagonists such as tamoxifen, which plays an important role in the treatment of breast cancer. While full agonists appear to arrest ER in certain conformation, weak agonists tend to maintain ER in equilibrium between different conformations, allowing cell-dependent differences in co-regulator repertoires to modulate the activity of ER in a cell-dependent manner (Tamrazi et al., Mol. Endocrinol. 17: 2593-2602, 2003). Interactions of ER with DNA are dynamic and include, but are not limited to, the degradation of ER by the proteasome (Reid et al., Mol Cell 11: 695-707, 2003). The degradation of ER with ligands provides an attractive treatment strategy for diseases or conditions that are estrogen-sensitive and/or resistant to available anti-hormonal treatments. ER signaling is crucial for the development and maintenance of female reproductive organs including breasts, ovulation and thickening of the endometrium. ER signaling also has a role in bone mass, lipid metabolism, cancers, etc. About 70% of breast cancers express ER-α (ER-α positive) and are dependent on estrogens for growth and survival. Other cancers also are thought to be dependent on ER-α signaling for growth and survival, such as for example ovarian and endometrial cancers. The ER-α antagonist tamoxifen has been used to treat early and advanced ER-α positive breast cancer in both pre- and post-menopausal women. Fulvestrant (FASLODEX™, AstraZeneca) a steroid-based ER antagonist is used to treat breast cancer in women which have progressed despite therapy with tamoxifen (Howell A. (2006) Endocr Relat Cancer; 13:689-706; U.S. Pat. No. 6,774,122; U.S. Pat. No. 7,456,160; U.S. Pat. No. 8,329,680; U.S. Pat. No. 8,466,139). Steroidal and non-steroidal aromatase inhibitors are also used to treat cancers in humans. In some embodiments, the steroidal and non-steroidal aromatase inhibitors block the production of estrogen from androstenedione and testosterone in post-menopausal women, thereby blocking ER dependent growth in the cancers. In addition to these anti-hormonal agents, progressive ER positive breast cancer is treated in some cases with a variety of other chemotherapeutics, such as for example, the anthracylines, platins, taxanes. In some cases, ER positive breast cancers that harbor genetic amplication of the ERB-B/HER2 tyrosine kinase receptor are treated with the monoclonal antibody trastuzumab (Herceptin™) or the small molecule pan-ERB-B inhibitor lapatinib. Despite this battery of anti-hormonal, chemotherapeutic and small-molecule and antibody-based targeted therapies, many women with ER-α positive breast develop progressive metastatic disease and are in need of new therapies. Importantly, the majority of ER positive tumors that progress on existing anti-hormonal, as well as and other therapies, are thought to remain dependent on ER-α for growth and survival. Thus, there is a need for new ER-α targeting agents that have activity in the setting of metastatic disease and acquired resistance. In one aspect, described herein are compounds that are selective estrogen receptor modulators (SERMs). In specific embodiments, the SERMs described herein are selective estrogen receptor degraders (SERDs). In some embodiments, in cell-based assays the compounds described herein result in a reduction in steady state ER-α levels (i.e. ER degradation) and are useful in the treatment of estrogen sensitive diseases or conditions and/or diseases or conditions that have developed resistant to anti-hormonal therapies.

Given the central role of ER-α in breast cancer development and progression, compounds disclosed herein are useful in the treatment of breast cancer, either alone or in combination with other agent agents that can modulate other critical pathways in breast cancer, including but not limited to those that target IGF1R, EGFR, CDK 4/6, erB-B2 and 3, the PI3K/AKT/mTOR axis, HSP90, PARP or histone deacetylases.

Given the central role of ER-α in breast cancer development and progression, compounds disclosed herein are useful in the treatment of breast cancer, either alone or in combination with other agent used to treat breast cancer, including but not limited to aromatase inhibitors, anthracylines, platins, nitrogen mustard alkylating agents, taxanes. Illustrative agent used to treat breast cancer, include, but are not limited to, paclitaxel, anastrozole, exemestane, cyclophosphamide, epirubicin, fulvestrant, letrozole, gemcitabine, trastuzumab, pegfilgrastim, filgrastim, tamoxifen, docetaxel, toremifene, vinorelbine, capecitabine, ixabepilone, as well as others described herein.

ER-related diseases or conditions include ER-α dysfunction is associated with cancer (bone cancer, breast cancer, lung cancer, colorectal cancer, endometrial cancer, prostate cancer, ovarian and uterine cancer), central nervous system (CNS) defects (alcoholism, migraine), cardiovascular system defects (aortic aneurysm, susceptibility to myocardial infarction, aortic valve sclerosis, cardiovascular disease, coronary artery disease, hypertension), hematological system defects (deep vein thrombosis), immune and inflammation diseases (Graves' Disease, arthritis, multiple sclerosis, cirrhosis), susceptibility to infection (hepatitis B, chronic liver disease), metabolic defects (bone density, cholestasis, hypospadias, obesity, osteoarthritis, osteopenia, osteoporosis), neurological defects (Alzheimer's disease, Parkinson's disease, migraine, vertigo), psychiatric defects (anorexia nervosa, attention deficit hyperactivity disorder (ADHD), dementia, major depressive disorder, psychosis) and reproductive defects (age of menarche, endometriosis, infertility.

In some embodiments, compounds disclosed herein are used in the treatment of an estrogen receptor dependent or estrogen receptor mediated disease or condition in mammal.

In some embodiments, the estrogen receptor dependent or estrogen receptor mediated disease or condition is selected from cancer, central nervous system (CNS) defects, cardiovascular system defects, hematological system defects, immune and inflammation diseases, susceptibility to infection, metabolic defects, neurological defects, psychiatric defects and reproductive defects.

In some embodiments, the estrogen receptor dependent or estrogen receptor mediated disease or condition is selected from bone cancer, breast cancer, lung cancer, colorectal cancer, endometrial cancer, prostate cancer, ovarian cancer, uterine cancer, alcoholism, migraine, aortic aneurysm, susceptibility to myocardial infarction, aortic valve sclerosis, cardiovascular disease, coronary artery disease, hypertension, deep vein thrombosis, Graves' Disease, arthritis, multiple sclerosis, cirrhosis, hepatitis B, chronic liver disease, bone density, cholestasis, hypospadias, obesity, osteoarthritis, osteopenia, osteoporosis, Alzheimer's disease, Parkinson's disease, migraine, vertigo, anorexia nervosa, attention deficit hyperactivity disorder (ADHD), dementia, major depressive disorder, psychosis, age of menarche, endometriosis, and infertility.

In some embodiments, compounds disclosed herein are used to treat cancer in a mammal. In some embodiments, the cancer is breast cancer, ovarian cancer, endometrial cancer, prostate cancer, or uterine cancer. In some embodiments, the cancer is breast cancer, lung cancer, ovarian cancer, endometrial cancer, prostate cancer, or uterine cancer. In some embodiments, the cancer is breast cancer. In some embodiments, the cancer is a hormone dependent cancer. In some embodiments, the cancer is an estrogen receptor dependent cancer. In some embodiments, the cancer is an estrogen-sensitive cancer. In some embodiments, the cancer is resistant to anti-hormonal treatment. In some embodiments, the cancer is an estrogen-sensitive cancer or an estrogen receptor dependent cancer that is resistant to anti-hormonal treatment. In some embodiments, the cancer is a hormone-sensitive cancer or a hormone receptor dependent cancer that is resistant to anti-hormonal treatment. In some embodiments, anti-hormonal treatment includes treatment with at least one agent selected from tamoxifen, fulvestrant, steroidal aromatase inhibitors, and non-steroidal aromatase inhibitors.

In some embodiments, compounds disclosed herein are used to treat hormone receptor positive metastatic breast cancer in a postmenopausal woman with disease progression following anti-estrogen therapy.

In some embodiments, compounds disclosed herein are used to treat a hormonal dependent benign or malignant disease of the breast or reproductive tract in a mammal. In some embodiments, the benign or malignant disease is breast cancer.

In some embodiments, the compound used in any of the methods described herein is an estrogen receptor degrader; is an estrogen receptor antagonist; has minimal or negligible estrogen receptor agonist activity; or combinations thereof.

In some embodiments, methods of treatment with compounds described herein include a treatment regimen that includes administering radiation therapy to the mammal.

In some embodiments, methods of treatment with compounds described herein include administering the compound prior to or following surgery.

In some embodiments, methods of treatment with compounds described herein include administering to the mammal at least one additional anti-cancer agent.

In some embodiments, compounds disclosed herein are used to treat cancer in a mammal, wherein the mammal is chemotherapy-naïve.

In some embodiments, compounds disclosed herein are used in the treatment of cancer in a mammal. In some embodiments, compounds disclosed herein are used to treat cancer in a mammal, wherein the mammal is being treated for cancer with at least one anti-cancer agent. In one embodiment, the cancer is a hormone refractory cancer.

In some embodiments, compounds disclosed herein are used in the treatment or prevention of diseases or conditions of the uterus in a mammal. In some embodiments, the disease or condition of the uterus is leiomyoma, uterine leiomyoma, endometrial hyperplasia, or endometriosis. In some embodiments, the disease or condition of the uterus is a cancerous disease or condition of the uterus. In some other embodiments, the disease or condition of the uterus is a non-cancerous disease or condition of the uterus.

In some embodiments, compounds disclosed herein are used in the treatment of endometriosis in a mammal.

In some embodiments, compounds disclosed herein are used in the treatment of leiomyoma in a mammal. In some embodiments, the leiomyoma is a uterine leiomyoma, esophageal leiomyoma, cutaneous leiomyoma, or small bowel leiomyoma. In some embodiments, compounds disclosed herein are used in the treatment of fibroids in a mammal.

In some embodiments, compounds disclosed herein are used in the treatment of uterine fibroids in a mammal.

### Compound of Formula (A), (B), or (C)

The Compound of Formula (A), (B), or (C), including pharmaceutically acceptable salts, prodrugs, active metabolites and pharmaceutically acceptable solvates thereof, is an estrogen receptor modulator (ERM). In specific embodiments, the compound is an estrogen receptor degrader. In specific embodiments, the compound is an estrogen receptor antagonist. In specific embodiments, the compound is an estrogen receptor degrader and estrogen receptor antagonist with minimal or no estrogen receptor agonist activity.

In some embodiments, compounds disclosed herein are estrogen receptor degraders and estrogen receptor antagonists that exhibit: minimal or no estrogen receptor agonism; and/or anti-proliferative activity against breast cancer, ovarian cancer, endometrial cancer, cervical cancer cell lines; and/or maximal anti-proliferative efficacy against breast cancer, ovarian cancer, endometrial cancer, cervical cell lines in-vitro; and/or maximal anti-proliferative efficacy against patient-derived breast cancer, patient-derived ovarian cancer, patient-derived endometrial cancer, patient-derived cervical cell lines in-vitro; and/or minimal agonism in the human endometrial (Ishikawa) cell line; and/or minimal or no agonism in the human endometrial (Ishikawa) cell line; and/or minimal or no agonism in the immature rat uterine assay in-vivo; and/or inverse agonism in the immature rat uterine assay in-vivo; and/or anti-tumor activity in breast cancer, ovarian cancer, endometrial cancer, cervical cancer cell lines in xenograft assays in-vivo or other rodent models of these cancers; and/or anti-tumor activity in patient-derived breast cancer, patient-derived ovarian cancer, patient-derived endometrial cancer, or patient-derived cervical cancer cell lines in xenograft assays in-vivo.

In some embodiments, compounds described herein have reduced or minimal interaction with the hERG (the human Ether-à-go-go-Related Gene) channel and/or show a reduced potential for QT prolongation and/or a reduced risk of ventricular tachyarrhythmias like torsades de pointes.

In some embodiments, the compound of Formula (A), (B), or (C), has reduced or minimal potential to access the hypothalamus and/or have reduced or minimal potential to modulate the Hypothalamic-Pituitary-Ovarian (HPO) axis and/or show a reduced potential to cause hyper-stimulation of the ovaries and/or show a reduced potential for ovary toxicity.

In some embodiments, the compound of Formula (A), (B), or (C), for use in the treatment of a disease or condition in a pre-menopausal woman have reduced or minimal potential to access the hypothalamus and/or have reduced or minimal potential to modulate the Hypothalamic-Pituitary-Ovarian (HPO) axis and/or show a reduced potential to cause hyper-stimulation of the ovaries and/or show a reduced potential for ovary toxicity. In some embodiments, the disease or condition in the pre-menopausal woman is endometriosis. In some embodiments, the disease or condition in the pre-menopausal woman is an uterine disease or condition.

In one aspect, described herein is a compound of Formula (A), or a pharmaceutically acceptable salt, solvate or prodrug thereof:

In one aspect, the estrogen receptor modulator compound for use in the methods and compositions described herein is a compound of Formula (A), or a pharmaceutically acceptable salt, or solvate thereof:

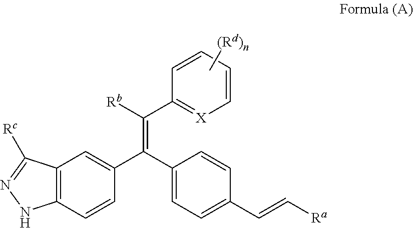

wherein,

R^{a} is —CO₂H or a 5-membered heterocycle selected from the group consisting of

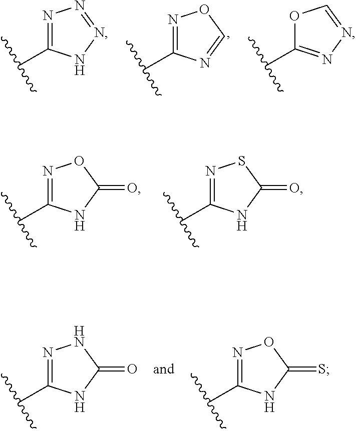

R^{b} is C₁-C₆alkyl or C₃-C₆cycloalkyl;

R^{c} is H or F;

each R^{d} is independently selected from H, halogen, —CN, —OR^{e}, —NHR^{e}, —NR^{e}R^{f}, —SR^{e}, —S(═O)R^{f}, —S(═O)₂R^{f}, C₁-C₆alkyl, and C₁-C₆fluoroalkyl;

each R^{e} is independently selected from H, —C(═O)R^{f}, —C(═O)OR^{f}, —C(═O)NHR^{f}, C₁-C₆alkyl, C₁-C₆fluoroalkyl, C₃-C₆cycloalkyl, substituted or unsubstituted monocyclic C₂-C₆heterocycloalkyl, substituted or unsubstituted phenyl, and substituted or unsubstituted monocyclic heteroaryl;

each R^{f} is independently selected from C₁-C₆alkyl, C₁-C₆fluoroalkyl, C₃-C₆cycloalkyl, substituted or unsubstituted monocyclic C₂-C₆heterocycloalkyl, substituted or unsubstituted phenyl, and substituted or unsubstituted monocyclic heteroaryl;

X is CH or N;

n is 0, 1, or 2.

In some embodiments, R^{a} is —CO₂H.

In some embodiments, R^{a} is a 5-membered heterocycle selected from the group consisting of

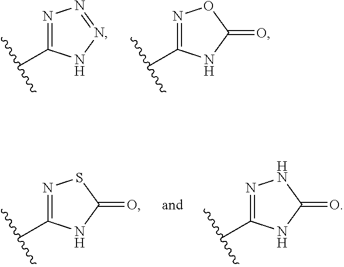 [SMILES: *c1nnn[nH]1 ; *c1noc(=O)[nH]1 ; *c1nsc(=O)[nH]1 ; *c1n[nH]c(=O)[nH]1]

In some embodiments, R^{c} is H. In some embodiments, R^{c} is F.

In some embodiments, R^{b} is —CH₃, —CH₂CH₃, cyclopropyl, or cyclobutyl. In some embodiments, R^{b} is —CH₂CH₃. In some embodiments, R^{b} is cyclobutyl.

In some embodiments X is CH. In some embodiments X is N.

In some embodiments, n is 1 or 2. In some embodiments, n is 1. In some embodiments, n is 2. In some embodiments, n is 0.

In some embodiments, each R^{d} is independently selected from H, F, Cl, —CN, —OH, —OCH₃, —OCH₂CH₃, —S(═O)₂CH₃, —CH₃, —CH₂H₃, and —CF₃. In some embodiments, each R^{d} is independently selected from H, F, Cl, —CN, —OH, —OCH₃, —OCH₂CH₃, —CH₃, —CH₂H₃, and —CF₃.

In some embodiments, the estrogen receptor modulator compound of Formula (A) has the following structure of Formula (A-1), or a pharmaceutically acceptable salt, or solvate thereof:

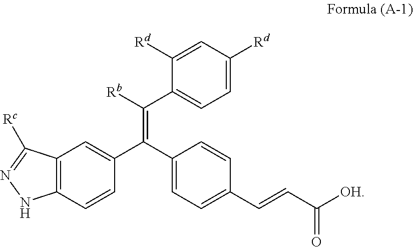

In some embodiments, the estrogen receptor modulator (ERM) compound of Formula (A) is a compound described in Table 1, or a pharmaceutically acceptable salt, or solvate thereof:

TABLE 1

| ERM | | |
|---|---|---|
| Compound | | |
| No. | Name | Structure |
| 1-1 | (E)-3-(4-((E)-2-(2,4-dichlorophenyl)-1- (1H-indazol-5-yl)but-1-en-1- yl)phenyl)acrylic acid | 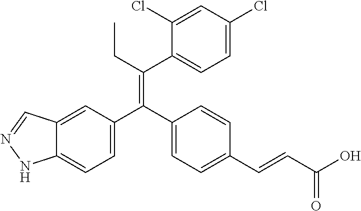 [SMILES: CC/C(=C(/c1ccc(/C=C/C(=O)O)cc1)c1ccc2[nH]ncc2c1)c1ccc(Cl)cc1Cl] |
| 1-2 | (E)-3-(4-((E)-2-(2,4-difluorophenyl)-1- (1H-indazol-5-yl)but-1-en-1- yl)phenyl)acrylic acid | 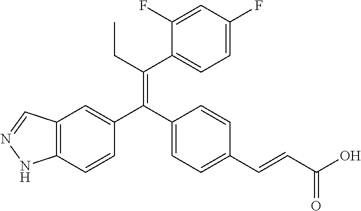 [SMILES: CC/C(=C(/c1ccc(/C=C/C(=O)O)cc1)c1ccc2[nH]ncc2c1)c1ccc(F)cc1F] |
| 1-3 | (E)-3-(4-((E)-2-(2-chloro-4- fluorophenyl)-1-(1H-indazol-5-yl)-but- 1-en-1-yl)phenyl)acrylic acid | 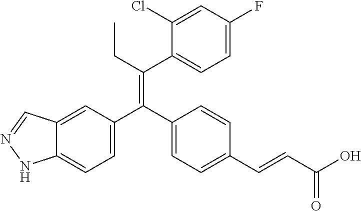 [SMILES: CC/C(=C(/c1ccc(/C=C/C(=O)O)cc1)c1ccc2[nH]ncc2c1)c1ccc(F)cc1Cl] |
| 1-4 | (E)-3-(4-((E)-2-(2-chloro-4- fluorophenyl)-1-(3-fluoro-1H-indazol- 5-yl)but-1-en-1-yl)phenyl)acrylic acid | 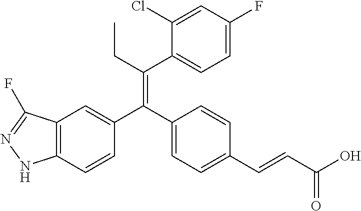 [SMILES: CC/C(=C(/c1ccc(/C=C/C(=O)O)cc1)c1ccc2[nH]nc(F)c2c1)c1ccc(F)cc1Cl] |
| 1-5 | (E)-3-(4-((E)-2-(2-chloro-4- fluorophenyl)-2-cyclobutyl-1-(1H- indazol-5-yl)vinyl)phenyl)acrylic acid | 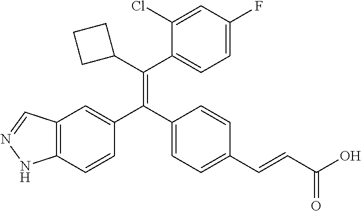 [SMILES: O=C(O)/C=C/c1ccc(/C(=C(\c2ccc(F)cc2Cl)C2CCC2)c2ccc3[nH]ncc3c2)cc1] |
| 1-6 | (E)-3-(4-((E)-2-(2-chloro-4- fluorophenyl)-2-cyclobutyl-1-(3- fluoro-1H-indazol-5- yl)vinyl)phenyl)acrylic acid | 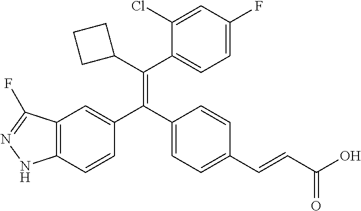 [SMILES: O=C(O)/C=C/c1ccc(/C(=C(\c2ccc(F)cc2Cl)C2CCC2)c2ccc3[nH]nc(F)c3c2)cc1] |
| 1-7 | (E)-3-(4-((E)-2-(4-chloro-2- cyanophenyl)-2-cyclobutyl-1-(3-fluoro- 1H-indazol-5-yl)vinyl)phenyl)acrylic acid | 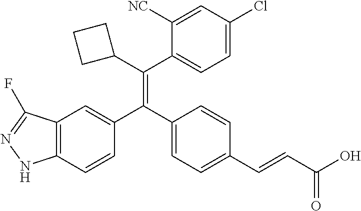 [SMILES: N#Cc1cc(Cl)ccc1/C(=C(\c1ccc(/C=C/C(=O)O)cc1)c1ccc2[nH]nc(F)c2c1)C1CCC1] |
| 1-8 | (E)-3-(4-((E)-2-(2-chloro-4- methoxyphenyl)-2-cyclobutyl-1-(3- fluoro-1H-indazol-5- yl)vinyl)phenyl)acrylic acid | 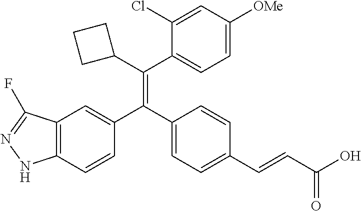 [SMILES: COc1ccc(/C(=C(\c2ccc(/C=C/C(=O)O)cc2)c2ccc3[nH]nc(F)c3c2)C2CCC2)c(Cl)c1] |
| 1-9 | (E)-3-(4-((E)-2-(3-chloro-5- (trifluoromethyl)pyridin-2-yl)-2- cyclobutyl-1-(3-fluoro-1H-indazol-5- yl)vinyl)phenyl)acrylic acid | 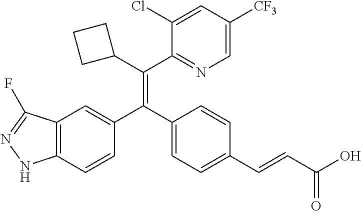 [SMILES: O=C(O)/C=C/c1ccc(/C(=C(\c2ncc(C(F)(F)F)cc2Cl)C2CCC2)c2ccc3[nH]nc(F)c3c2)cc1] |
| 1-10 | (E)-3-(4-((E)-2-(2-cyano-4- (trifluoromethyl)phenyl)-2-cyclobutyl- 1-(3-fluoro-1H-indazol-5- yl)vinyl)phenyl)acrylic acid | 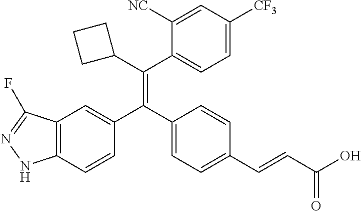 [SMILES: N#Cc1cc(C(F)(F)F)ccc1/C(=C(\c1ccc(/C=C/C(=O)O)cc1)c1ccc2[nH]nc(F)c2c1)C1CCC1] |
| 1-11 | (E)-3-(4-((E)-2-(2-chloro-4- cyanophenyl)-2-cyclobutyl-1-(3-fluoro- 1H-indazol-5-yl)vinyl)phenyl)acrylic acid | 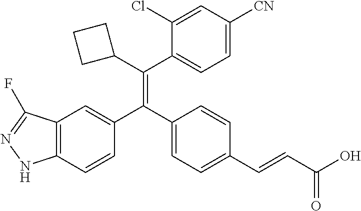 [SMILES: N#Cc1ccc(/C(=C(\c2ccc(/C=C/C(=O)O)cc2)c2ccc3[nH]nc(F)c3c2)C2CCC2)c(Cl)c1] |
| 1-12 | 3-((E)-4-((E)-2-(2-chloro-4- methoxyphenyl)-2-cyclobutyl-1-(3- fluoro-1H-indazol-5-yl)vinyl)styryl)- 1,2,4-oxadiazol-5(4H)-one | 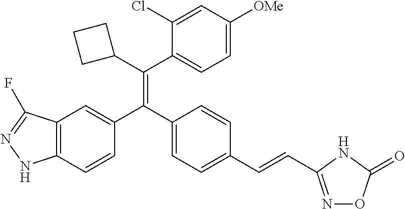 [SMILES: COc1ccc(/C(=C(\c2ccc(/C=C/c3noc(=O)[nH]3)cc2)c2ccc3[nH]nc(F)c3c2)C2CCC2)c(Cl)c1] |

In another aspect, the estrogen receptor modulator compound for use in the methods and compositions described herein is a compound of Formula (B) or a pharmaceutically acceptable salt, or solvate thereof:

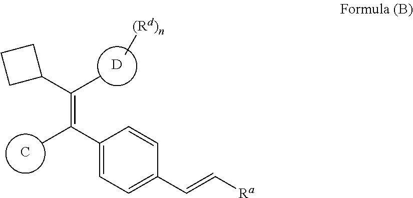

wherein,

R^{a} is —CO₂H or a 5-membered heterocycle selected from the group consisting of

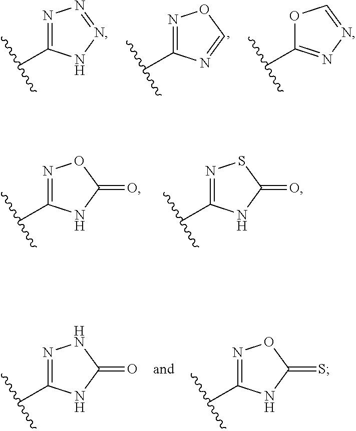

ring C is

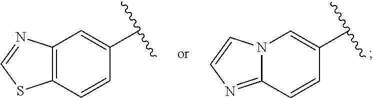 [SMILES: *c1ccc2scnc2c1 ; *c1ccc2nccn2c1]

ring D is phenyl or thienyl;

each R^{d} is independently selected from H, halogen, —CN, —OR^{e}, —NHR^{e}, —NR^{e}R^{f}, —SR^{e}, —S(═O)R^{f}, —S(═O)₂R^{f}, C₁-C₆alkyl, and C₁-C₆fluoroalkyl;

each R^{e} is independently selected from H, —C(═O)R^{f}, —C(═O)OR^{f}, —C(═O)NHR^{f}, C₁-C₆alkyl, C₁-C₆fluoroalkyl, C₃-C₆cycloalkyl, substituted or unsubstituted monocyclic C₂-C₆heterocycloalkyl, substituted or unsubstituted phenyl, and substituted or unsubstituted monocyclic heteroaryl;

each R^{f} is independently selected from C₁-C₆alkyl, C₁-C₆fluoroalkyl, C₃-C₆cycloalkyl, substituted or unsubstituted monocyclic C₂-C₆heterocycloalkyl, substituted or unsubstituted phenyl, and substituted or unsubstituted monocyclic heteroaryl;

n is 0, 1, or 2.

In some embodiments, ring C is

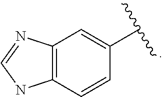 [SMILES: *c1ccc2[nH]cnc2c1]

In some embodiments, ring C is

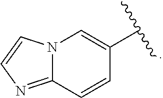 [SMILES: *c1ccc2nccn2c1]

In some embodiments,

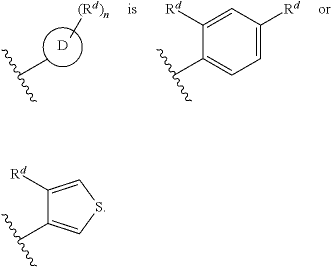

In some embodiments,

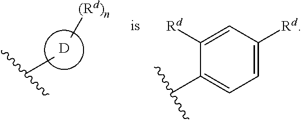

In some embodiments,

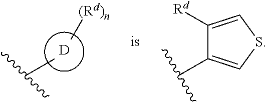

In some embodiments, R^{a} is —CO₂H.

In some embodiments, n is 1 or 2. In some embodiments, n is 1. In some embodiments, n is 2. In some embodiments, n is 0. In some embodiments, each R^{d} is independently selected from H, F, Cl, —CN, —OH, —OCH₃, —OCH₂CH₃, —S(═O)₂CH₃, —CH₃, —CH₂H₃, and —CF₃. In some embodiments, each R^{d} is independently selected from H, F, Cl, —CN, —OH, —OCH₃, —OCH₂CH₃, —CH₃, —CH₂H₃, and —CF₃.

In some embodiments, the estrogen receptor modulator (ERM) compound of Formula (B) is a compound described in Table 2, or a pharmaceutically acceptable salt, or solvate thereof:

TABLE 2

| ERM | | |
|---|---|---|
| Compound | | |
| No. | Name | Structure |
| 2-1 | (E)-3-(4-((E)-1-(benzo[d]thiazol-5- yl)-2-(2-chloro-4-methoxyphenyl)- 2-cyclobutylvinyl)phenyl)acrylic acid | 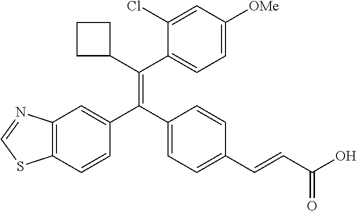 [SMILES: COc1ccc(/C(=C(\c2ccc(/C=C/C(=O)O)cc2)c2ccc3scnc3c2)C2CCC2)c(Cl)c1] |
| 2-2 | (E)-3-(4-((E)-1-(benzo[d]thiazol-5- yl)-2-cyclobutyl-2-(4- methylthiophen-3- yl)vinyl)phenyl)acrylic acid | 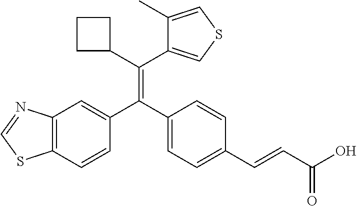 [SMILES: Cc1cscc1/C(=C(\c1ccc(/C=C/C(=O)O)cc1)c1ccc2scnc2c1)C1CCC1] |
| 2-3 | (E)-3-(4-((E)-1-(benzo[d]thiazol-5- yl)-2-(2-cyano-4-methoxyphenyl)- 2-cyclobutylvinyl)phenyl)acrylic acid | 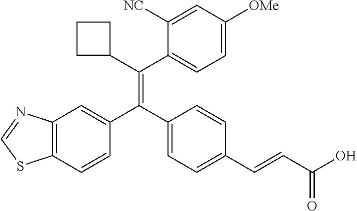 [SMILES: COc1ccc(/C(=C(\c2ccc(/C=C/C(=O)O)cc2)c2ccc3scnc3c2)C2CCC2)c(C#N)c1] |
| 2-4 | (E)-3-(4-((E)-1-(benzo[d]thiazol-5- yl)-2-(2-cyano-4- (trifluoromethyl)phenyl)-2- cyclobutylvinyl)phenyl)acrylic acid | 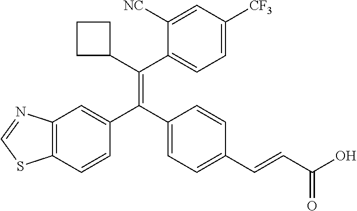 [SMILES: N#Cc1cc(C(F)(F)F)ccc1/C(=C(\c1ccc(/C=C/C(=O)O)cc1)c1ccc2scnc2c1)C1CCC1] |
| 2-5 | (E)-3-(4-((E)-2-(2-chloro-4- methoxyphenyl)-2-cyclobutyl-1- (imidazo[1,2-a]pyridin-6- yl)vinyl)phenyl)acrylic acid | 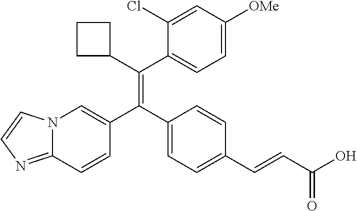 [SMILES: COc1ccc(/C(=C(\c2ccc(/C=C/C(=O)O)cc2)c2ccc3nccn3c2)C2CCC2)c(Cl)c1] |

In another aspect, the estrogen receptor modulator compound for use in the methods and compositions described herein is a compound of Formula (C), or a pharmaceutically acceptable salt, or solvate thereof:

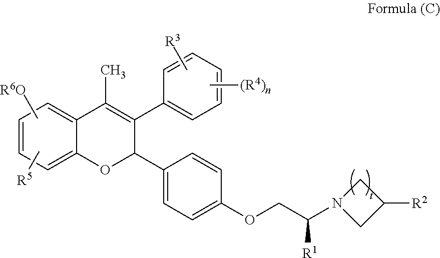

wherein,

R¹ is H, C₁-C₄alkyl, or C₁-C₄fluoroalkyl;

R² is H, F, C₁-C₄alkyl or C₁-C₄fluoroalkyl;

R³ is H, halogen, —CN, —OR⁶, —NHR⁶, —NR⁶R⁷, —SR⁶, —S(═O)R⁷, —S(═O)₂R⁷, C₁-C₄alkyl, or C₁-C₄fluoroalkyl;

each R⁴ is independently selected from H, halogen, —CN, —OH, C₁-C₆alkyl, C₁-C₄fluoroalkyl, C₁-C₄fluoroalkoxy, and C₁-C₄alkoxy;

each R⁵ is H, F, Cl, —OH, —CH₃, —CF₃, or —OCH₃;

each R⁶ is independently selected from H, —C(═O)R⁷, —C(═O)OR⁷, —C(═O)NHR⁷, C₁-C₆alkyl, C₁-C₆fluoroalkyl, C₃-C₆cycloalkyl, substituted or unsubstituted C₂-C₆heterocycloalkyl, substituted or unsubstituted phenyl, and substituted or unsubstituted monocyclic heteroaryl;

each R⁷ is independently selected from C₁-C₆alkyl, C₁-C₆fluoroalkyl, substituted or unsubstituted C₃-C₆cycloalkyl, substituted or unsubstituted monocyclic C₂-C₆heterocycloalkyl, substituted or unsubstituted phenyl, and substituted or unsubstituted monocyclic heteroaryl;

n is 0, 1, or 2;

t is 1 or 2.

In some embodiments, R¹ is H, —CH₃, —CH₂F, —CHF₂, or —CF₃.

In some embodiments, each R² is independently F, —CH₃, —CH₂CH₃, —CH₂F, —CHF₂, —CF₃, —CHFCH₃, —CH₂CH₂F, —CH₂CHF₂, —CH₂CF₃, —CH₂CH₂CF₃, —CH₂CH₂CH₂CF₃, —CHCH₃CF₃, —CH(CF₃)₂, or —CF(CH₃)₂. In some embodiments, each R² is independently F, —CH₃, —CH₂F, —CHF₂, or —CF₃. In some embodiments, each R² is independently —CH₃, —CH₂F, —CHF₂, or —CF₃. In some embodiments, each R² is independently —CH₂F. In some embodiments, each R² is independently —CH₃.

In some embodiments, t is 1. In some embodiments, t is 2.

In some embodiments, R³ is —OR⁶. In some embodiments, R³ is —OH.

In some embodiments, each R⁴ is independently selected from H, F, Cl, —OH, —CH₃, —CF₃, or —OCH₃.

In some embodiments, each R⁵ is independently selected from H and F.

In some embodiments, each R⁶ is independently selected from H, —C(═O)R⁷, —C(═O)OR⁷, —C(═O)NHR⁷, C₁-C₆alkyl, C₁-C₆fluoroalkyl, and substituted or unsubstituted phenyl. In some embodiments, each R⁶ is H.

In some embodiments, each R⁷ is independently selected from C₁-C₆alkyl, C₁-C₆fluoroalkyl, and substituted or unsubstituted phenyl.

In some embodiments,

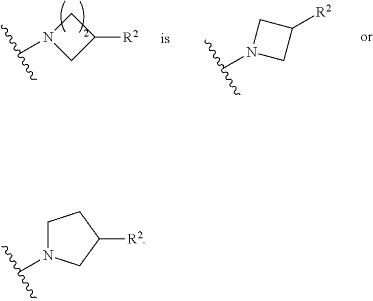

In some embodiments,

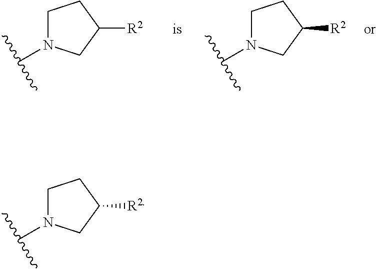

In some embodiments,

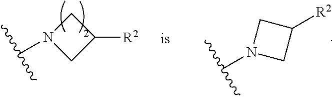

In some embodiments,

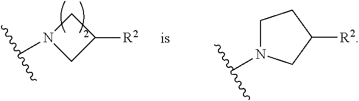

In some embodiments,

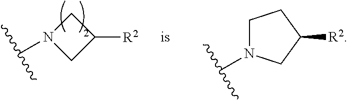

In some embodiments,

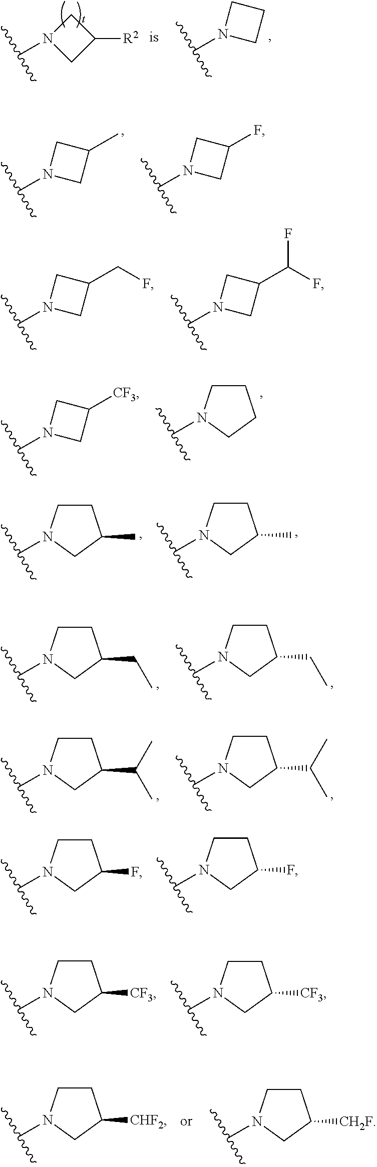

In some embodiments,

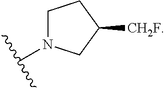

In some embodiments,

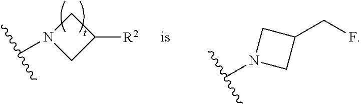

In some embodiments,

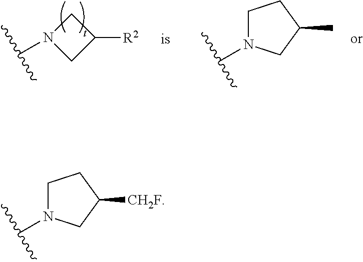

In some embodiments,

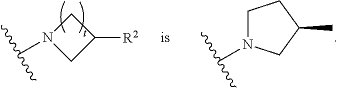

In some embodiments,

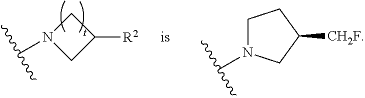

In some embodiments, the estrogen receptor modulator compound of Formula (C) has the following structure of Formula (C-1), or a pharmaceutically acceptable salt, or solvate thereof:

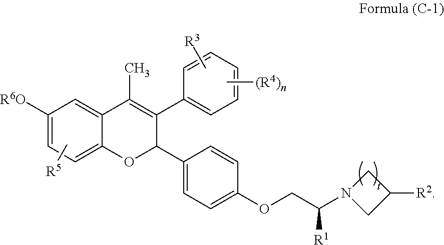

In some embodiments, the estrogen receptor modulator compound of Formula (C) has the following structure of Formula (C-2), or a pharmaceutically acceptable salt, or solvate thereof:

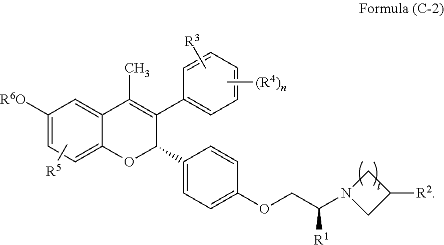

In some embodiments, the estrogen receptor modulator compound of Formula (C) has the following structure of Formula (C-3), or a pharmaceutically acceptable salt, or solvate thereof:

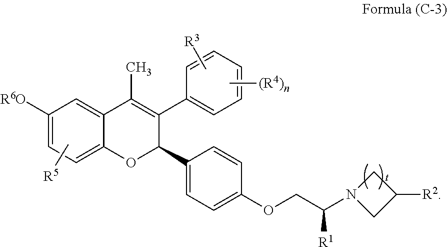

In some embodiments, the estrogen receptor modulator compound of Formula (C) has the following structure of Formula (C-4), or a pharmaceutically acceptable salt, or solvate thereof:

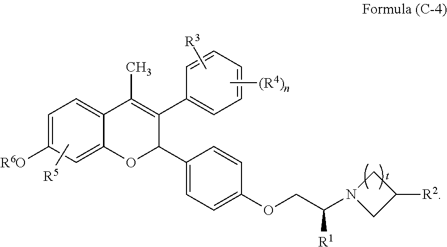

In some embodiments, the estrogen receptor modulator compound of Formula (C) has the following structure of Formula (C-5), or a pharmaceutically acceptable salt, or solvate thereof:

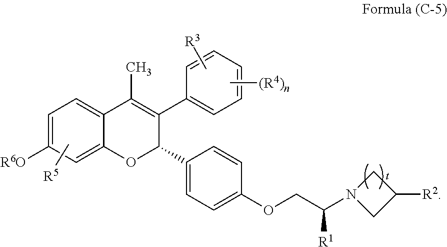

In some embodiments, the estrogen receptor modulator compound of Formula (C) has the following structure of Formula (C-6), or a pharmaceutically acceptable salt, or solvate thereof:

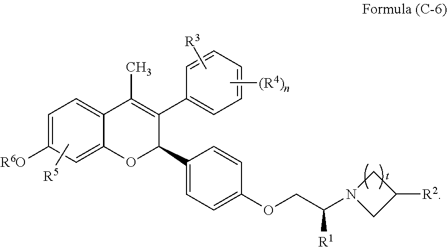

In some embodiments,

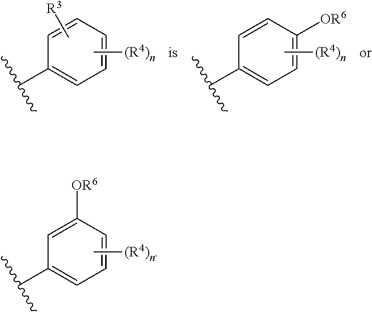

In some embodiments, the estrogen receptor modulator (ERM) compound of Formula (C) is a compound described in Table 3, or a pharmaceutically acceptable salt, or solvate thereof:

TABLE 3

| ERM | | |
|---|---|---|
| Compound | | |
| No. | Name | Structure |
| 4-1 | 3-(3-hydroxyphenyl)-4- methyl-2-(4-(2-((R)-3- methylpyrrolidin-1- yl)ethoxy)phenyl)-2H- chromen-6-ol | 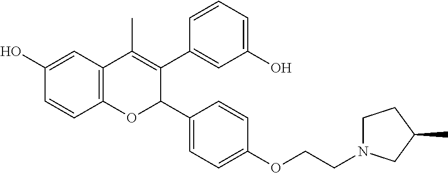 [SMILES: CC1=C(c2cccc(O)c2)C(c2ccc(OCCN3CC[C@@H](C)C3)cc2)Oc2ccc(O)cc21] |
| 4-2 | (S)-3-(3-hydroxyphenyl)-4- methyl-2-(4-(2-((R)-3- methylpyrrolidin-1- yl)ethoxy)phenyl)-2H- chromen-6-ol | 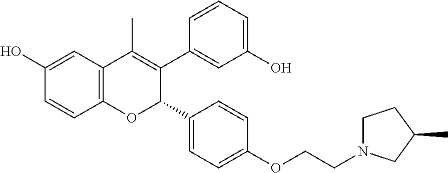 [SMILES: CC1=C(c2cccc(O)c2)[C@H](c2ccc(OCCN3CC[C@@H](C)C3)cc2)Oc2ccc(O)cc21] |
| 4-3 | (R)-3-(3-hydroxyphenyl)-4- methyl-2-(4-(2-((R)-3- methylpyrrolidin-1- yl)ethoxy)phenyl)-2H- chromen-6-ol | 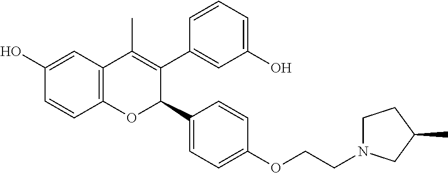 [SMILES: CC1=C(c2cccc(O)c2)[C@@H](c2ccc(OCCN3CC[C@@H](C)C3)cc2)Oc2ccc(O)cc21] |
| 4-4 | 3-(3-hydroxyphenyl)-4- methyl-2-(4-((S)-2- (pyrrolidin-1- yl)propoxy)phenyl)-2H- chromen-6-ol | 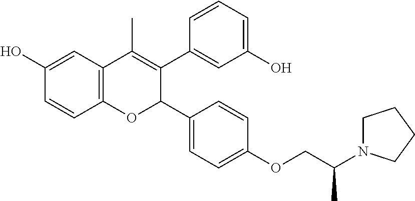 [SMILES: CC1=C(c2cccc(O)c2)C(c2ccc(OC[C@H](C)N3CCCC3)cc2)Oc2ccc(O)cc21] |
| 4-5 | (S)-3-(3-hydroxyphenyl)-4- methyl-2-(4-((S)-2- (pyrrolidin-1- yl)propoxy)phenyl)-2H- chromen-6-ol | 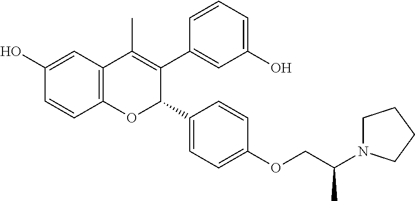 [SMILES: CC1=C(c2cccc(O)c2)[C@H](c2ccc(OC[C@H](C)N3CCCC3)cc2)Oc2ccc(O)cc21] |
| 4-6 | (R)-3-(3-hydroxyphenyl)-4- methyl-2-(4-((S)-2- (pyrrolidin-1- yl)propoxy)phenyl)-2H- chromen-6-ol | 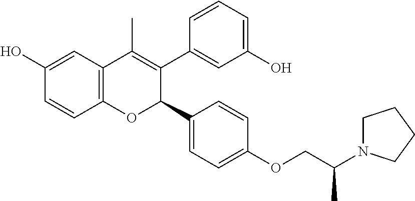 [SMILES: CC1=C(c2cccc(O)c2)[C@@H](c2ccc(OC[C@H](C)N3CCCC3)cc2)Oc2ccc(O)cc21] |
| 4-7 | 3-(3-hydroxyphenyl)-4- methyl-2-(4-((S)-2-((R)-3- methylpyrrolidin-1- yl)propoxy)phenyl)-2H- chromen-7-ol | 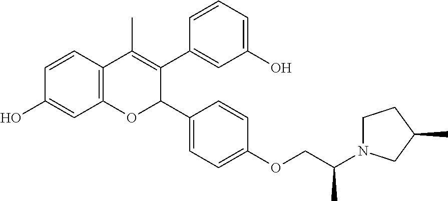 [SMILES: CC1=C(c2cccc(O)c2)C(c2ccc(OC[C@H](C)N3CC[C@@H](C)C3)cc2)Oc2cc(O)ccc21] |
| 4-8 | (S)-3-(3-hydroxyphenyl)-4- methyl-2-(4-((S)-2-((R)-3- methylpyrrolidin-1- yl)propoxy)phenyl)-2H- chromen-7-ol | 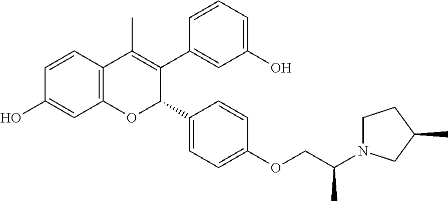 [SMILES: CC1=C(c2cccc(O)c2)[C@H](c2ccc(OC[C@H](C)N3CC[C@@H](C)C3)cc2)Oc2cc(O)ccc21] |
| 4-9 | (R)-3-(3-hydroxyphenyl)-4- methyl-2-(4-((S)-2-((R)-3- methylpyrrolidin-1- yl)propoxy)phenyl)-2H- chromen-7-ol | 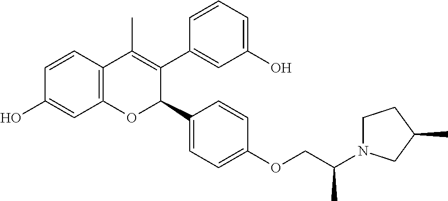 [SMILES: CC1=C(c2cccc(O)c2)[C@@H](c2ccc(OC[C@H](C)N3CC[C@@H](C)C3)cc2)Oc2cc(O)ccc21] |
| 4-10 | 3-(4-hydroxyphenyl)-4- methyl-2-(4-((S)-2-((R)-3- methylpyrrolidin-1- yl)propoxy)phenyl)-2H- chromen-6-ol | 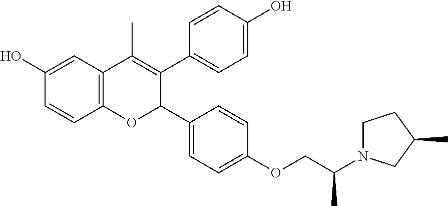 [SMILES: CC1=C(c2ccc(O)cc2)C(c2ccc(OC[C@H](C)N3CC[C@@H](C)C3)cc2)Oc2ccc(O)cc21] |
| 4-11 | (S)-3-(4-hydroxyphenyl)-4- methyl-2-(4-((S)-2-((R)-3- methylpyrrolidin-1- yl)propoxy)phenyl)-2H- chromen-6-ol | 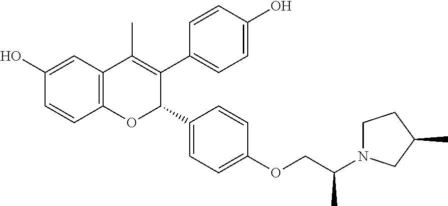 [SMILES: CC1=C(c2ccc(O)cc2)[C@H](c2ccc(OC[C@H](C)N3CC[C@@H](C)C3)cc2)Oc2ccc(O)cc21] |
| 4-12 | (R)-3-(4-hydroxyphenyl)-4- methyl-2-(4-((S)-2-((R)-3- methylpyrrolidin-1- yl)propoxy)phenyl)-2H- chromen-6-ol | 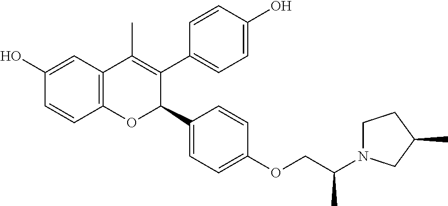 [SMILES: CC1=C(c2ccc(O)cc2)[C@@H](c2ccc(OC[C@H](C)N3CC[C@@H](C)C3)cc2)Oc2ccc(O)cc21] |
| 4-13 | 3-(3-hydroxyphenyl)-4- methyl-2-(4-((S)-2-((R)-3- methylpyrrolidin-1- yl)propoxy)phenyl)-2H- chromen-6-ol | 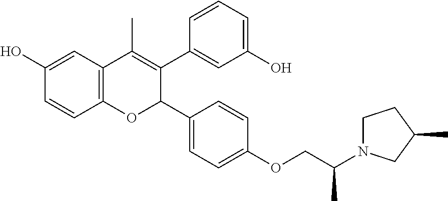 [SMILES: CC1=C(c2cccc(O)c2)C(c2ccc(OC[C@H](C)N3CC[C@@H](C)C3)cc2)Oc2ccc(O)cc21] |
| 4-14 | (S)-3-(3-hydroxyphenyl)-4- methyl-2-(4-((S)-2-((R)-3- methylpyrrolidin-1- yl)propoxy)phenyl)-2H- chromen-6-ol | 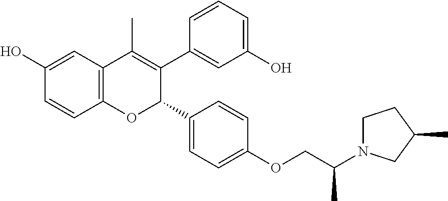 [SMILES: CC1=C(c2cccc(O)c2)[C@H](c2ccc(OC[C@H](C)N3CC[C@@H](C)C3)cc2)Oc2ccc(O)cc21] |
| 4-15 | (R)-3-(3-hydroxyphenyl)-4- methyl-2-(4-((S)-2-((R)-3- methylpyrrolidin-1- yl)propoxy)phenyl)-2H- chromen-6-ol | 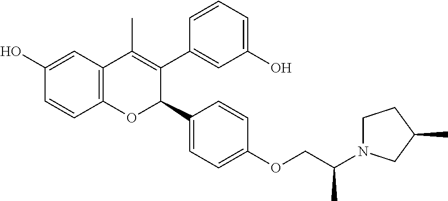 [SMILES: CC1=C(c2cccc(O)c2)[C@@H](c2ccc(OC[C@H](C)N3CC[C@@H](C)C3)cc2)Oc2ccc(O)cc21] |
| 4-16 | 2-(4-((S)-2-((R)-3- (fluoromethyl)pyrrolidin-1- yl)propoxy)phenyl)-3-(3- hydroxyphenyl)-4-methyl-2H- chromen-6-ol | 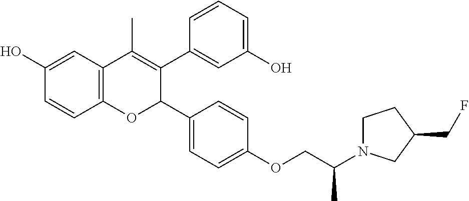 [SMILES: CC1=C(c2cccc(O)c2)C(c2ccc(OC[C@H](C)N3CC[C@@H](CF)C3)cc2)Oc2ccc(O)cc21] |
| 4-17 | (S)-2-(4-((S)-2-((R)-3- (fluoromethyl)pyrrolidin-1- yl)propoxy)phenyl)-3-(3- hydroxyphenyl)-4-methyl-2H- chromen-6-ol | 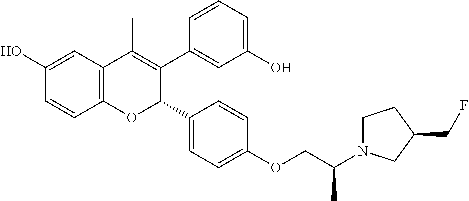 [SMILES: CC1=C(c2cccc(O)c2)[C@H](c2ccc(OC[C@H](C)N3CC[C@@H](CF)C3)cc2)Oc2ccc(O)cc21] |
| 4-18 | (R)-2-(4-((S)-2-((R)-3- (fluoromethyl)pyrrolidin-1- yl)propoxy)phenyl)-3-(3- hydroxyphenyl)-4-methyl-2H- chromen-6-ol | 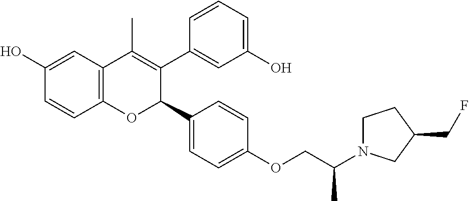 [SMILES: CC1=C(c2cccc(O)c2)[C@@H](c2ccc(OC[C@H](C)N3CC[C@@H](CF)C3)cc2)Oc2ccc(O)cc21] |
| 4-19 | 2-(4-(2-((R)-3- (fluoromethyl)pyrrolidin-1- yl)ethoxy)phenyl)-3-(3- hydroxyphenyl)-4-methyl-2H- chromen-6-ol | 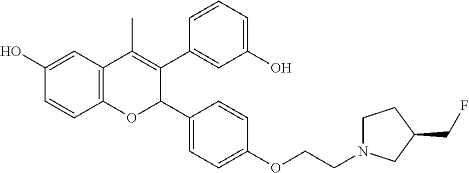 [SMILES: CC1=C(c2cccc(O)c2)C(c2ccc(OCCN3CC[C@@H](CF)C3)cc2)Oc2ccc(O)cc21] |
| 4-20 | (S)-2-(4-(2-((R)-3- (fluoromethyl)pyrrolidin-1- yl)ethoxy)phenyl)-3-(3- hydroxyphenyl)-4-methyl-2H- chromen-6-ol | 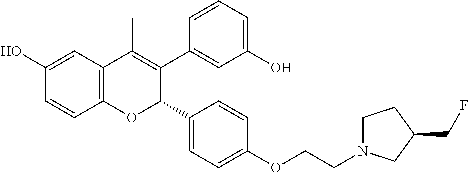 [SMILES: CC1=C(c2cccc(O)c2)[C@H](c2ccc(OCCN3CC[C@@H](CF)C3)cc2)Oc2ccc(O)cc21] |
| 4-21 | (R)-2-(4-(2-((R)-3- (fluoromethyl)pyrrolidin-1- yl)ethoxy)phenyl)-3-(3- hydroxyphenyl)-4-methyl-2H- chromen-6-ol | 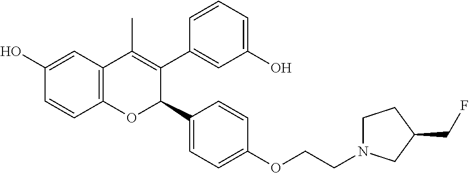 [SMILES: CC1=C(c2cccc(O)c2)[C@@H](c2ccc(OCCN3CC[C@@H](CF)C3)cc2)Oc2ccc(O)cc21] |
| 4-22 | 2-(4-(2-((R)-3- (fluoromethyl)pyrrolidin-1- yl)ethoxy)phenyl)-3-(4- fluorophenyl)-4-methyl-2H- chromen-6-ol | 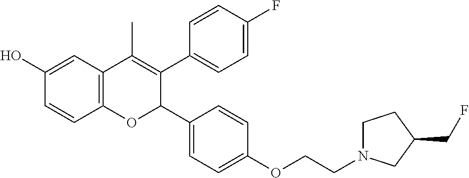 [SMILES: CC1=C(c2ccc(F)cc2)C(c2ccc(OCCN3CC[C@@H](CF)C3)cc2)Oc2ccc(O)cc21] |
| 4-23 | (S)-2-(4-(2-((R)-3- (fluoromethyl)pyrrolidin-1- yl)ethoxy)phenyl)-3-(4- fluorophenyl)-4-methyl-2H- chromen-6-ol | 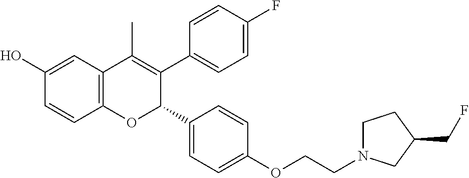 [SMILES: CC1=C(c2ccc(F)cc2)[C@H](c2ccc(OCCN3CC[C@@H](CF)C3)cc2)Oc2ccc(O)cc21] |
| 4-24 | (R)-2-(4-(2-((R)-3- (fluoromethyl)pyrrolidin-1- yl)ethoxy)phenyl)-3-(4- fluorophenyl)-4-methyl-2H- chromen-6-ol | 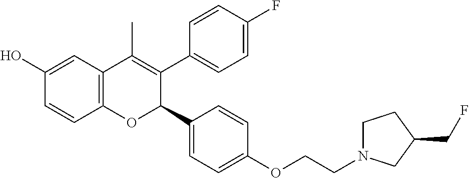 [SMILES: CC1=C(c2ccc(F)cc2)[C@@H](c2ccc(OCCN3CC[C@@H](CF)C3)cc2)Oc2ccc(O)cc21] |
| 4-25 | 2-(4-((S)-2-((R)-3- (fluoromethyl)pyrrolidin-1- yl)propoxy)phenyl)-3-(4- fluorophenyl)-4-methyl-2H- chromen-6-ol | 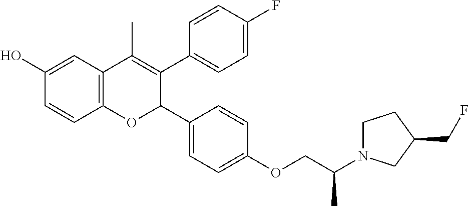 [SMILES: CC1=C(c2ccc(F)cc2)C(c2ccc(OC[C@H](C)N3CC[C@@H](CF)C3)cc2)Oc2ccc(O)cc21] |
| 4-26 | (S)-2-(4-((S)-2-((R)-3- (fluoromethyl)pyrrolidin-1- yl)propoxy)phenyl)-3-(4- fluorophenyl)-4-methyl-2H- chromen-6-ol | 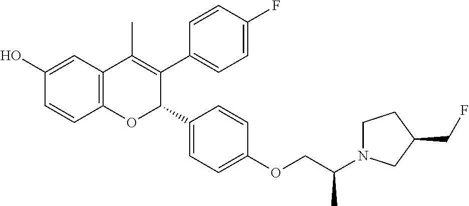 [SMILES: CC1=C(c2ccc(F)cc2)[C@H](c2ccc(OC[C@H](C)N3CC[C@@H](CF)C3)cc2)Oc2ccc(O)cc21] |
| 4-27 | (R)-2-(4-((S)-2-((R)-3- (fluoromethyl)pyrrolidin-1- yl)propoxy)phenyl)-3-(4- fluorophenyl)-4-methyl-2H- chromen-6-ol | 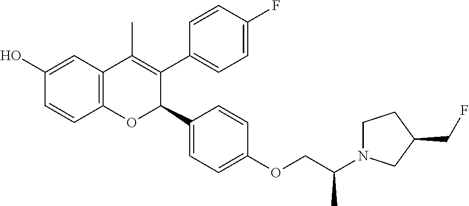 [SMILES: CC1=C(c2ccc(F)cc2)[C@@H](c2ccc(OC[C@H](C)N3CC[C@@H](CF)C3)cc2)Oc2ccc(O)cc21] |
| 4-28 | 2-(4-(2-(3- (difluoromethyl)azetidin-1- yl)ethoxy)phenyl)-3-(3- hydroxyphenyl)-4-methyl-2H- chromen-6-ol | 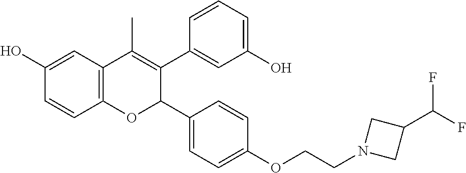 [SMILES: CC1=C(c2cccc(O)c2)C(c2ccc(OCCN3CC(C(F)F)C3)cc2)Oc2ccc(O)cc21] |
| 4-29 | (S)-2-(4-(2-(3- (difluoromethyl)azetidin-1- yl)ethoxy)phenyl)-3-(3- hydroxyphenyl)-4-methyl-2H- chromen-6-ol | 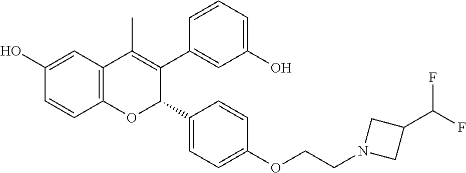 [SMILES: CC1=C(c2cccc(O)c2)[C@H](c2ccc(OCCN3CC(C(F)F)C3)cc2)Oc2ccc(O)cc21] |
| 4-30 | (R)-2-(4-(2-(3- (difluoromethyl)azetidin-1- yl)ethoxy)phenyl)-3-(3- hydroxyphenyl)-4-methyl-2H- chromen-6-ol | 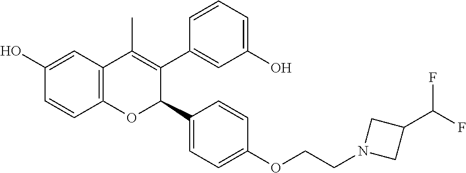 [SMILES: CC1=C(c2cccc(O)c2)[C@@H](c2ccc(OCCN3CC(C(F)F)C3)cc2)Oc2ccc(O)cc21] |
| 4-31 | 2-(4-(2-((R)-3- (fluoromethyl)pyrrolidin-1- yl)ethoxy)phenyl)-4-methyl- 3-phenyl-2H-chromen-6-ol | 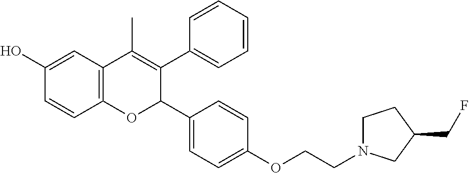 [SMILES: CC1=C(c2ccccc2)C(c2ccc(OCCN3CC[C@@H](CF)C3)cc2)Oc2ccc(O)cc21] |
| 4-32 | (S)-2-(4-(2-((R)-3- (fluoromethyl)pyrrolidin-1- yl)ethoxy)phenyl)-4-methyl- 3-phenyl-2H-chromen-6-ol | 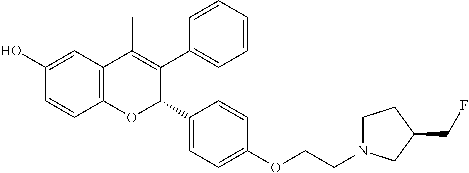 [SMILES: CC1=C(c2ccccc2)[C@H](c2ccc(OCCN3CC[C@@H](CF)C3)cc2)Oc2ccc(O)cc21] |
| 4-33 | (R)-2-(4-(2-((R)-3- (fluoromethyl)pyrrolidin-1- yl)ethoxy)phenyl)-4-methyl- 3-phenyl-2H-chromen-6-ol | 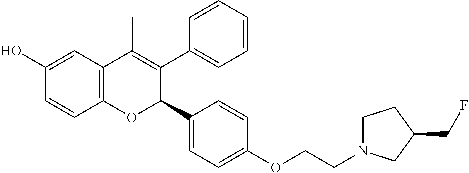 [SMILES: CC1=C(c2ccccc2)[C@@H](c2ccc(OCCN3CC[C@@H](CF)C3)cc2)Oc2ccc(O)cc21] |
| 4-34 | (S)-2-(4-(2-(3- (fluoromethyl)azetidin-1- yl)ethoxy)phenyl)-3-(3- hydroxyphenyl)-4-methyl-2H- chromen-6-ol | 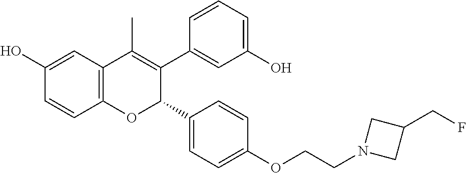 [SMILES: CC1=C(c2cccc(O)c2)[C@H](c2ccc(OCCN3CC(CF)C3)cc2)Oc2ccc(O)cc21] |
| 4-35 | (S)-2-(4-(2-(3- (fluoromethyl)azetidin-1- yl)ethoxy)phenyl)-3-(4- fluorophenyl)-4-methyl-2H- chromen-6-ol | 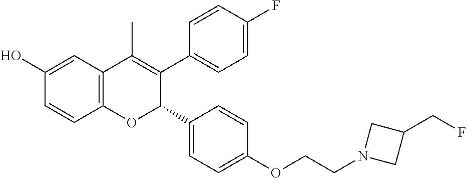 [SMILES: CC1=C(c2ccc(F)cc2)[C@H](c2ccc(OCCN3CC(CF)C3)cc2)Oc2ccc(O)cc21] |

Throughout the specification, groups and substituents thereof are chosen by one skilled in the field to provide stable moieties and compounds.

### Further Forms of Compounds

In one aspect, compounds described herein exist as a racemic mixture or in enantiomerically enriched or enantiomerically pure form. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In certain embodiments, compounds described herein are prepared as their individual stereoisomers by enzymatic resolution. In some embodiments, resolution of individual stereoisomers is carried out using a lipase or an esterase. In some embodiments, resolution of individual stereoisomers is carried out by lipase or esterase-catalyzed asymmetric deacylation. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

The methods and compositions described herein include the use of amorphous forms as well as crystalline forms (also known as polymorphs). In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. In some embodiments, the active entity is a phenolic compound as described herein. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like.

Prodrug forms of the herein described compounds, wherein the prodrug is metabolized in vivo to produce a compound of Formula (A), (B), or (C), as set forth herein are included within the scope of the claims. In some cases, some of the herein-described compounds may be a prodrug for another derivative or active compound.

In some embodiments, sites on the aromatic ring portion of compounds described herein are susceptible to various metabolic reactions. Incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, deuterium or an alkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms that are present in the compounds described herein is replaced with one or more deuterium atoms.

In additional or further embodiments, the compounds described herein are metabolized upon administration to an organism in need to produce a metabolite that is then used to produce a desired effect, including a desired therapeutic effect.

“Pharmaceutically acceptable,” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound described herein with acids. Pharmaceutically acceptable salts are also obtained by reacting a compound described herein with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid to form a salt such as, for example, a hydrochloric acid salt, a hydrobromic acid salt, a sulfuric acid salt, a phosphoric acid salt, a metaphosphoric acid salt, and the like; or with an organic acid to form a salt such as, for example, an acetic acid salt, a propionic acid salt, a hexanoic acid salt, a cyclopentanepropionic acid salt, a glycolic acid salt, a pyruvic acid salt, a lactic acid salt, a malonic acid salt, a succinic acid salt, a malic acid salt, a L-malic acid salt, a maleic acid salt, an oxalic acid salt, a fumaric acid salt, a trifluoroacetic acid salt, a tartaric acid salt, a L-tartaric acid salt, a citric acid salt, a benzoic acid salt, a 3-(4-hydroxybenzoyl)benzoic acid salt, a cinnamic acid salt, a mandelic acid salt, a methanesulfonic acid salt, an ethanesulfonic acid salt, a 1,2-ethanedisulfonic acid salt, a 2-hydroxyethanesulfonic acid salt, a benzenesulfonic acid salt, a toluenesulfonic acid salt, a 2-naphthalenesulfonic acid salt, a 4-methylbicyclo-[2.2.2]oct-2-ene-1-carboxylic acid salt, a glucoheptonic acid salt, a 4,4′-methylenebis-(3-hydroxy-2-ene-1-carboxylic acid) salt, a 3-phenylpropionic acid salt, a trimethylacetic acid salt, a tertiary butylacetic acid salt, a lauryl sulfuric acid salt, a gluconic acid salt, a glutamic acid salt, a hydroxynaphthoic acid salt, a salicylic acid salt, a stearic acid salt, a muconic acid salt, a butyric acid salt, a phenylacetic acid salt, a phenylbutyric acid salt, a valproic acid salt, and the like; (2) salts formed when an acidic proton present in the parent compound is replaced by a metal ion, e.g., an alkali metal ion (e.g. a lithium salt, a sodium salt, or a potassium salt), an alkaline earth ion (e.g. a magnesium salt, or a calcium salt), or an aluminum ion (e.g. an aluminum salt). In some cases, compounds described herein may coordinate with an organic base to form a salt, such as, but not limited to, an ethanolamine salt, a diethanolamine salt, a triethanolamine salt, a tromethamine salt, a N-methylglucamine salt, a dicyclohexylamine salt, or a tris(hydroxymethyl)methylamine salt. In other cases, compounds described herein may form salts with amino acids such as, but not limited to, an arginine salt, a lysine salt, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms.

### DEFINITIONS

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error.

An “alkyl” group refers to an aliphatic hydrocarbon group. The alkyl moiety may be branched or straight chain. The “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). Typical alkyl groups include, but are in no way limited to, methyl, ethyl, propyl, isopropyl, butyl, isobutyl, sec-butyl, tertiary butyl, pentyl, neopentyl, hexyl, and the like. In some embodiments, 1 or more hydrogen atoms of an alkyl are replaced with 1 or more deuterium atoms.

The term “halo” or, alternatively, “halogen” or “halide” means fluoro (F), chloro (Cl), bromo (Br) or iodo (I).

An “alkyl” group refers to an aliphatic hydrocarbon group. The alkyl moiety may be branched or straight chain. The “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). In one aspect the alkyl is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, sec-butyl, tertiary butyl, pentyl, neopentyl, hexyl, and the like. In some embodiments, 1 or more hydrogen atoms of an alkyl are replaced with 1 or more deuterium atoms.

An “alkoxy” group refers to a (alkyl)O— group, where alkyl is as defined herein.

The term “aromatic” refers to a planar ring having a delocalized π-electron system containing 4n+2 π electrons, where n is an integer. Aromatics are optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from heterocyclic rings in which the ring backbone contains at least one atom which is different from carbon.

As used herein, the term “aryl” refers to an aromatic ring wherein each of the atoms forming the ring is a carbon atom. In one aspect, an aryl is a phenyl or a naphthalenyl. In one aspect, an aryl is a phenyl. In one aspect, an aryl is a C₆-C₁₀ aryl. In some embodiments, 1 or more hydrogen atoms of an aryl are replaced with 1 or more deuterium atoms

The term “cycloalkyl” refers to a cyclic aliphatic hydrocarbon radical. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, and cyclooctyl. In one aspect, a cycloalkyl is a C₃-C₆ cycloalkyl.

The term “halo” or, alternatively, “halogen” or “halide” means fluoro (F), chloro (Cl), bromo (Br) or iodo (I). In some embodiments, halogen is F or Cl. In some embodiments, halogen is F.

The term “fluoroalkyl” refers to an alkyl in which one or more hydrogen atoms are replaced by a fluorine atom. In one aspect, a fluoralkyl is a C₁-C₆fluoroalkyl. In some embodiments, a fluoroalkyl is a monofluoroalkyl, wherein one hydrogen atom of the alkyl is replaced by a fluorine atom. In some embodiments, a fluoroalkyl is a difluoroalkyl, wherein two hydrogen atoms of the alkyl are replaced by a fluorine atom. In some embodiments, a fluoroalkyl is a trifluoroalkyl, wherein three hydrogen atom of the alkyl are replaced by a fluorine atom. In some embodiments, a fluoroalkyl is a monofluoroalkyl, difluoroalkyl, or trifluoroalkyl. In some embodiments, a monofluoroalkyl is —CH₂F, —CHF₂, —CF₃, —CHFCH₃, —CH₂CH₂F, —CH₂CHF₂, —CH₂CF₃, —CH₂CH₂CF₃, —CH₂CH₂CH₂CF₃, —CHCH₃CF₃, —CH(CF₃)₂, or —CF(CH₃)₂.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that the any ring does not contain two adjacent 0 or S atoms. Non-aromatic heterocyclic groups (also known as heterocycloalkyls) include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl. An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups may be C-attached (or C-linked) or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems.

The terms “heteroaryl” refers to an aryl group that includes one or more ring heteroatoms selected from nitrogen, oxygen and sulfur. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. In some embodiments, a heteroaryl contains 0-3 N atoms in the ring. In some embodiments, a heteroaryl contains 1-3 N atoms in the ring. In some embodiments, a heteroaryl contains 0-3 N atoms, 0-1 O atoms, and 0-1 S atoms in the ring. In some embodiments, monocyclic heteroaryl is a 5-membered or 6-membered heteroaryl.

A “heterocycloalkyl” or “heteroalicyclic” group refers to a cycloalkyl group wherein at least one of the carbon atoms of the cycloalkyl is replaced with nitrogen (unsubstituted or substituted, e.g. —NH—, —NR^{e}—), oxygen (—O—), or sulfur (e.g. —S—, —S(═O)— or —S(═O)₂—). The radicals may be fused with an aryl or heteroaryl. In some embodiments, the heterocycloalkyl is selected from oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, and indolinyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, nitro, haloalkyl, fluoroalkyl, fluoroalkoxy, and amino, including mono- and di-substituted amino groups, and the protected derivatives thereof. In some embodiments, optional substituents are independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The methods and formulations described herein include the use of N-oxides (if appropriate), crystalline forms (also known as polymorphs), or pharmaceutically acceptable salts of a compound having the structure of Formula (A), (B), or (C), as well as active metabolites of these compounds having the same type of activity. In some situations, compounds may exist as tautomers. All tautomers are included within the scope of the compounds presented herein. In specific embodiments, the compounds described herein exist in solvated forms with pharmaceutically acceptable solvents such as water, ethanol, and the like. In other embodiments, the compounds described herein exist in unsolvated form.

The term “enantiomeric ratio” refers to ratio of the percentage of one enantiomer in a mixture to that of the other. In some embodiments, compositions disclosed herein include a Formula (C-2) or Formula (C-5) with an enantiomeric ratio of at least 80%—(S):20%—(R), at least 85%—(S):15%—(R), at least 90%—(S):10%—(R), at least 95%—(S):5%—(R), at least 99%—(S):1%—(R), or greater than 99%—(S):1%—(R). In some embodiments, compositions described herein include enantiomerically pure compound of Formula (C-2) or Formula (C-5). In some embodiments, compositions disclosed herein include a Formula (C-3) or Formula (C-6) with an enantiomeric ratio of at least 80%—(R):20%—(S), at least 85%—(R):15%—(S), at least 90%—(R):10%—(S), at least 95%—(R):5%—(S), at least 99%—(R):1%—(S), or greater than 99%—(R):1%—(S). In some embodiments, compositions described herein include enantiomerically pure compound of Formula (C-3) or Formula (C-6).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist. In some embodiments, a modulator is a degrader.

“Selective estrogen receptor modulator” or “SERM” as used herein, refers to a molecule that differentially modulates the activity of estrogen receptors in different tissues. For example, in some embodiments, a SERM displays ER antagonist activity in some tissues and ER agonist activity in other tissues. In some embodiments, a SERM displays ER antagonist activity in some tissues and minimal or no ER agonist activity in other tissues. In some embodiments, a SERM displays ER antagonist activity in breast tissues, ovarian tissues, endometrial tissues, and/or cervical tissues but minimal or no ER agonist activity in uterine tissues. In some embodiments, a SERM displays ER degradation properties. In some embodiments, a SERM displays ER degradation properties in some tissues and no ER degradation properties in other tissues. In some embodiments, a SERM displays ER degradation and ER antagonist properties. In some embodiments, a SERM displays ER degradation and ER antagonist properties in some tissues and ER degradation but no ER agonist activity in other tissues. In some embodiments, a SERM displays ER degradation and ER antagonist properties in some tissues and ER degradation and ER antagonist properties but no ER degradation properties in other tissues. In some embodiments, a SERM displays ER degradation and ER antagonist properties in breast tissues, ovarian tissues, endometrial tissues, and/or cervical tissues but minimal or no ER degradation and/or ER antagonist properties in uterine tissues.

The term “antagonist” as used herein, refers to a small-molecule agent that binds to a nuclear hormone receptor and subsequently decreases the agonist induced transcriptional activity of the nuclear hormone receptor.

The term “agonist” as used herein, refers to a small-molecule agent that binds to a nuclear hormone receptor and subsequently increases nuclear hormone receptor transcriptional activity in the absence of a known agonist.

The term “inverse agonist” as used herein, refers to a small-molecule agent that binds to a nuclear hormone receptor and subsequently decreases the basal level of nuclear hormone receptor transcriptional activity that is present in the absence of a known agonist.

The term “degrader” as used herein, refers to a small molecule agent that binds to a nuclear hormone receptor and subsequently lowers the steady state protein levels of said receptor. In some embodiments, a degrader as described herein lowers steady state estrogen receptor levels by at least 10%, at least 20%, at least 30%, at least 40%, at least 50%, at least 60%, at least 65%, at least 70%, at least 75%, at least 80%, at least 85%, at least 90%, at least 95%, at least 96%, at least 97%, at least 98%, or at least 99%. In some embodiments, a degrader as described herein lowers steady state estrogen receptor levels by at least 65%. In some embodiments, a degrader as described herein lowers steady state estrogen receptor levels by at least 85%.

The term “selective estrogen receptor degrader” or “SERD” as used herein, refers to a small molecule agent that preferentially binds to estrogen receptors versus other receptors and subsequently lowers the steady state estrogen receptor levels.

The term “ER-dependent”, as used herein, refers to diseases or conditions that would not occur, or would not occur to the same extent, in the absence of estrogen receptors.

The term “ER-mediated”, as used herein, refers to diseases or conditions that would not occur in the absence of estrogen receptors but can occur in the presence of estrogen receptors.

The term “ER-sensitive”, as used herein, refers to diseases or conditions that would not occur, or would not occur to the same extent, in the absence of estrogens.

The term “cancer” as used herein refers to an abnormal growth of cells which tend to proliferate in an uncontrolled way and, in some cases, to metastasize (spread). The types of cancer include, but is not limited to, solid tumors (such as those of the bladder, bowel, brain, breast, endometrium, heart, kidney, lung, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

Additional non-limiting examples of cancers include, acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, lung cancer, non-small cell lung cancer, small cell lung cancer, Burkitt lymphoma, cutaneous T-cell lymphoma, Hodgkin lymphoma, non-Hodgkin lymphoma, lymphoma, Waldenström macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sézary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenström macroglobulinemia, Wilms tumor.

The term “breast cancer” as used herein refers to histologically or cytologically confirmed cancer of the breast. In some embodiments, the breast cancer is a carcinoma. In some embodiments, the breast cancer is an adenocarcinoma. In some embodiments, the breast cancer is a sarcoma.

The term “locally advanced breast cancer” refers to cancer that has spread from where it started in the breast to nearby tissue or lymph nodes, but not to other parts of the body.

The term “metastatic breast cancer” refers to cancer that has spread from the breast to other parts of the body, such as the bones, liver, lungs, or brain. Metastatic breast cancer is also referred to as stage IV breast cancer.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (A), (B), or (C), or a pharmaceutically acceptable salt thereof, and a co-agent, are both administered to a patient simultaneously in the form of a single entity or dosage. The term “non-fixed combination” means that the active ingredients, e.g. a compound of Formula (A), (B), or (C), or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, delaying progression of condition, relieving a condition caused by the disease or condition, or stopping the symptoms of the disease or condition either prophylactically and/or therapeutically. In some embodiments, treatment includes extending progression-free survival. In some embodiments, treatment includes reducing the relative risk of disease progression compared to other treatment options. In some embodiments, other treatment options include but are not limited to hormonal treatments (e.g., anti-estrogen therapy, such as tamoxifen and/or fulvestrant or aromatase therapy).

The term “progression-free survival” is the amount of time during and after the treatment of a disease, such as cancer, that a patient lives with the disease but it does not get worse. In a clinical trial, measuring progression-free survival is one way to see how well a treatment works.

The term “metastasis-free survival” or “MFS” refers to the percentage of subjects in a study who have survived without cancer spread for a defined period of time or death. MFS is usually reported as time from the beginning of treatment in the study. MFS is reported for an individual or a study population. In some embodiments, the increase in the metastasis-free survival is about 1 month, about 2 months, about 2 months, about 3 months, about 4 months, about 5 months, about 6 months, about 7 months, about 8 months, about 10 months, about 11 months, about 12 months, about 13 months, about 14 months, about 15 months, about 16 months, about 17 months, about 18 months, about 19 months, about 20 months, or greater than 20 months.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions/Formulations

In some embodiments, the estrogen receptor modulator compounds as described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound of Formula (A), (B), or (C), with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to a mammal.

The pharmaceutical compositions will include at least one compound of Formula (A), (B), or (C), or a pharmaceutically acceptable salt thereof, as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds described herein exist in unsolvated form or in solvated forms with pharmaceutically acceptable solvents such as water, ethanol, and the like.

The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, enteric coated formulations, pulsatile release formulations, multiparticulate formulations, and mixed immediate and controlled release formulations.

In some embodiments, the compound of Formula (A), (B), or (C), or a pharmaceutically acceptable salt thereof, is administered systemically.

In some embodiments, the compound of Formula (A), (B), or (C), or a pharmaceutically acceptable salt thereof, is administered orally. All formulations for oral administration are in dosages suitable for such administration. In some embodiments, the solid dosage forms disclosed herein are in the form of a tablet, a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. In still other embodiments, the pharmaceutical formulation is in the form of a tablet. In still other embodiments, the pharmaceutical formulation is in the form of a suspension tablet, a fast-melt tablet, a bite-disintegration tablet, a rapid-disintegration tablet, an effervescent tablet, or a caplet. In other embodiments, pharmaceutical formulation is in the form of a capsule.

In some embodiments, the pharmaceutical solid oral dosage forms are formulated to provide a controlled release of the active compound. Controlled release profiles include, for example, sustained release, prolonged release, pulsatile release, and delayed release profiles.

In one aspect, liquid formulation dosage forms for oral administration are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002).

For buccal or sublingual administration, the compositions optionally take the form of tablets, lozenges, or gels formulated in a conventional manner.

In one aspect, a compound of Formula (A), (B), or (C), or a pharmaceutically acceptable salt thereof, is formulated into a pharmaceutical composition suitable for intramuscular, subcutaneous, or intravenous injection. Parenteral injections involve either bolus injection and/or continuous infusion.

In some embodiments, the compound of Formula (A), (B), or (C), or a pharmaceutically acceptable salt thereof, is administered intravenously. In some embodiments, the compound of Formula (A), (B), or (C), or a pharmaceutically acceptable salt thereof, is administered subcutaneously.

In some embodiments, the compound of Formula (A), (B), or (C), or a pharmaceutically acceptable salt thereof, is administered topically. In such embodiments, the compound of Formula (A), (B), or (C), or a pharmaceutically acceptable salt thereof, is formulated into a variety of topically administrable compositions, such as solutions, suspensions, lotions, gels, pastes, shampoos, scrubs, rubs, smears, medicated sticks, medicated bandages, balms, creams or ointments. In some embodiments, the compound of Formula (A), (B), or (C), or a pharmaceutically acceptable salt thereof, is administered topically to the skin of mammal. In some embodiments, the compound of Formula (A), (B), or (C), is prepared as a transdermal dosage form.

In another aspect is the use of a compound of Formula (A), (B), or (C), or a pharmaceutically acceptable salt thereof, in the manufacture of a medicament for treating a disease, disorder or conditions in which the activity of estrogen receptors contributes to the pathology and/or symptoms of the disease or condition. In one aspect, the disease or condition is any of the diseases or conditions specified herein.

A therapeutically effective amount of an estrogen receptor modulator, or a pharmaceutically acceptable salt thereof, can vary widely depending on the severity of the disease, the age and relative health of the subject, and other factors.

### Methods of Dosing and Treatment Regimens

In one embodiment, the compounds of Formula (A), (B), or (C), or a pharmaceutically acceptable salt thereof, are used in the preparation of medicaments for the treatment of diseases or conditions in a mammal that would benefit from a reduction of estrogen receptor activity. Methods for treating any of the diseases or conditions described herein in a mammal in need of such treatment, involves administration of pharmaceutical compositions that include at least one compound of Formula (A), (B), or (C), or a pharmaceutically acceptable salt thereof, or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound of Formula (A), (B), or (C), or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but can nevertheless be determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 2000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound of Formula (A), (B), or (C), or a pharmaceutically acceptable salt thereof, described herein are from about 0.01 to about 10 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

In some embodiments, an estrogen receptor modulator, or a pharmaceutically acceptable salt thereof, is administered orally to postmenopausal women.

In some embodiments, an estrogen receptor modulator of Formula (A), (B), or (C), or a pharmaceutically acceptable salt thereof, is administered daily to the patient. In some embodiments, an estrogen receptor modulator of Formula (A), (B), or (C), or a pharmaceutically acceptable salt thereof, is administered every other day to the patient. In some embodiments, an estrogen receptor modulator of Formula (A), (B), or (C), or a pharmaceutically acceptable salt thereof, is administered daily, every other day, every third day, every fourth day, every fifth day, every sixth day, once a week, once every two weeks, once every three weeks, or once a month to the patient.

In some embodiments, an estrogen receptor modulator of Formula (A), (B), or (C), or a pharmaceutically acceptable salt thereof, is administered orally to the patient on a continuous daily dosing schedule.

The term “continuous daily dosing schedule” refers to the administration of an estrogen receptor modulator, or a pharmaceutically acceptable salt thereof, daily without any drug holidays. In some embodiments, a continuous daily dosing schedule comprises administration of an estrogen receptor modulator, or a pharmaceutically acceptable salt thereof, everyday at roughly the same time each day.

In some embodiments, about 10 mg per day to about 4000 mg per day of an estrogen receptor modulator of Formula (A), (B), or (C), or a pharmaceutically acceptable salt thereof, is administered to the patient. In some embodiments, about 10 mg per day to about 3000 mg per day of an estrogen receptor modulator of Formula (A), (B), or (C), or a pharmaceutically acceptable salt thereof, is administered to the patient. In some embodiments, about 10 mg per day to about 2000 mg per day of an estrogen receptor modulator of Formula (A), (B), or (C), or a pharmaceutically acceptable salt thereof, is administered to the patient. In some embodiments, about 10 mg per day to about 1000 mg per day of an estrogen receptor modulator of Formula (A), (B), or (C), or a pharmaceutically acceptable salt thereof, is administered to the patient. In some embodiments, about 20 mg per day to about 2000 mg per day of an estrogen receptor modulator of Formula (A), (B), or (C), or a pharmaceutically acceptable salt thereof, is administered to the patient.

In some embodiments, about 10 mg per day, about 15 mg per day, about 20 mg per day, about 25 mg per day, about 30 mg per day, about 35 mg per day, about 35 mg per day, about 40 mg per day, about 45 mg per day, about 50 mg per day, about 55 mg per day, about 60 mg per day, about 65 mg per day, about 70 mg per day, about 75 mg per day, about 80 mg per day, about 85 mg per day, about 90 mg per day, about 100 mg per day, about 150 mg per day, about 200 mg per day, about 250 mg per day, about 300 mg per day, about 350 mg per day, about 400 mg per day, about 450 mg per day, about 500 mg per day, about 550 mg per day, about 600 mg per day, about 650 mg per day, about 700 mg per day, about 750 mg per day, about 800 mg per day, 850 mg per day, about 900 mg per day, about 950 mg per day, about 1000 mg per day, about 1050 mg per day, about 1100 mg per day, about 1150 mg per day, about 1200 mg per day, about 1250 mg per day, about 1300 mg per day, about 1350 mg per day, about 1400 mg per day, about 1450 mg per day, about 1500 mg per day, about 1550 mg per day, about 1600 mg per day, about 1650 mg per day, about 1700 mg per day, about 1750 mg per day, about 1800 mg per day, about 1850 mg per day, about 1900 mg per day, about 1950 mg per day, about 2000 mg per day, about 2050 mg per day, about 2100 mg per day, about 2150 mg per day, about 2200 mg per day, about 2250 mg per day, about 2300 mg per day, about 2350 mg per day, about 2400 mg per day, about 2450 mg per day, about 2500 mg per day, about 2550 mg per day, about 2600 mg per day, about 2650 mg per day, about 2700 mg per day, about 2750 mg per day, about 2800 mg per day, about 2850 mg per day, about 2900 mg per day, about 2950 mg per day, or about 3000 mg per day of an estrogen receptor modulator of Formula (A), (B), or (C), or a pharmaceutically acceptable salt thereof, is administered to the patient.

In some embodiments, about 600 mg per day of an estrogen receptor modulator of Formula (A), (B), or (C), or a pharmaceutically acceptable salt thereof, is administered to the patient.

In some embodiments, about 1000 mg per day of an estrogen receptor modulator of Formula (A), (B), or (C), or a pharmaceutically acceptable salt thereof, is administered to the patient.

In some embodiments, about 100 mg per day, about 200 mg per day, about 300 mg per day, about 400 mg per day, about 500 mg per day, about 600 mg per day, about 700 mg per day, about 800 mg per day, about 900 mg per day, about 1000 mg per day, about 1100 mg per day, about 1200 mg per day, about 1300 mg per day, about 1400 mg per day, about 1500 mg per day, about 1600 mg per day, about 1700 mg per day, about 1800 mg per day, about 1900 mg per day, or about 2000 mg per day of an estrogen receptor modulator of Formula (A), (B), or (C), or a pharmaceutically acceptable salt thereof, is administered to the patient.

In one embodiment, the desired daily dose is conveniently presented in a single dose or in divided doses administered simultaneously (or over a short period of time) or at appropriate intervals, for example as two, three, four or more sub-doses per day. In some embodiments, the desired daily dose is conveniently presented in divided doses that are administered simultaneously (or over a short period of time) once a day. In some embodiments, the desired daily dose is conveniently presented in divided doses that are administered in equal portions twice-a-day, three times a day, or more than three times a day.

In some embodiments, the desired daily amount of an estrogen receptor modulator of Formula (A), (B), or (C), or a pharmaceutically acceptable salt thereof, that is administered to a patient is administered once a day.

In some embodiments, the daily amount of an estrogen receptor modulator of Formula (A), (B), or (C), or a pharmaceutically acceptable salt thereof, administered to a patient is administered twice a day in evenly divided doses.

In some embodiments, the daily amount of an estrogen receptor modulator of Formula (A), (B), or (C), or a pharmaceutically acceptable salt thereof, administered to a patient is administered three times a day in evenly divided doses.

In some embodiments, the daily amount of an estrogen receptor modulator of Formula (A), (B), or (C), or a pharmaceutically acceptable salt thereof, administered to a patient is administered more than three times a day in evenly divided doses.

In certain embodiments wherein improvement in the status of the breast cancer in the patient is not observed, the daily dose of an estrogen receptor modulator of Formula (A), (B), or (C), or a pharmaceutically acceptable salt thereof, is increased. In some embodiments, a once-a-day dosing schedule is changed to a twice-a-day dosing schedule. In some embodiments, a three times a day dosing schedule is employed to increase the amount of an estrogen receptor modulator, or a pharmaceutically acceptable salt thereof, that is administered.

In some embodiments, an estrogen receptor modulator of Formula (A), (B), or (C), or a pharmaceutically acceptable salt thereof, is administered to a patient in the fasted state. In some embodiments, an estrogen receptor modulator of Formula (A), (B), or (C), or a pharmaceutically acceptable salt thereof, is administered to a patient in the fed state.

In some embodiments, the amount of an estrogen receptor modulator of Formula (A), (B), or (C), or a pharmaceutically acceptable salt thereof, that is given to a patient varies depending upon factors such as, but not limited to, condition and severity of the breast cancer, and the identity (e.g., weight) of the woman.

### Combination Treatments

In certain instances, it is appropriate to administer at least one compound of Formula (A), (B), or (C), or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant may have minimal therapeutic benefit, but in combination with another therapeutic agent, the overall therapeutic benefit to the patient is enhanced). Or, in some embodiments, the benefit experienced by a patient is increased by administering one of the compounds described herein with another therapeutic agent (which also includes a therapeutic regimen) that also has therapeutic benefit.

In one specific embodiment, a compound of Formula (A), (B), or (C), or a pharmaceutically acceptable salt thereof, is co-administered with a second therapeutic agent, wherein the compound of Formula (A), (B), or (C), or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient may simply be additive of the two therapeutic agents or the patient may experience a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens can be determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound of Formula (A), (B), or (C), or a pharmaceutically acceptable salt thereof, is initiated prior to, during, or after treatment with a second agent described herein, and continues until any time during treatment with the second agent or after termination of treatment with the second agent. It also includes treatments in which a compound of Formula (A), (B), or (C), or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compound of Formula (A), (B), or (C), or a pharmaceutically acceptable salt thereof, as well as combination therapies, is administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### Exemplary Agents for Use in Combination Therapy

In some embodiments, methods for treatment of estrogen receptor-dependent or estrogen receptor-mediated conditions or diseases, such as proliferative disorders, including cancer, comprises administration to a mammal a compound of Formula (A), (B), or (C), or a pharmaceutically acceptable salt thereof, in combination with at least one additional therapeutic agent.

In some embodiments, a compound of Formula (A), (B), or (C), or a pharmaceutically acceptable salt thereof, is used in combination with one or more additional therapeutically active agents selected from: corticosteroids, anti-emetic agents, analgesics, anti-cancer agents, anti-inflammatories, kinase inhibitors, antibodies, HSP90 inhibitors, histone deacetylase (HDAC) inhibitors, modulators of the immune system, PD-1 inhibitors, poly ADP-ribose polymerase (PARP) inhibitors, and aromatase inhibitors.

In certain instances, it is appropriate to administer at least one compound of Formula (A), (B), or (C), or a pharmaceutically acceptable salt thereof, in combination with one or more other therapeutic agents. In certain embodiments, the one or more other therapeutic agents is an anti-cancer agent(s).

In some embodiments, a compound of Formula (A), (B), or (C), or a pharmaceutically acceptable salt thereof, is used in combination with an aromatase inhibitor, a phosphoinositide 3-kinase (PI3K)/mTOR pathway inhibitor, a CDK 4/6 inhibitor, a HER-2 inhibitor, an EGFR inhibitor, a PD-1 inhibitor, poly ADP-ribose polymerase (PARP) inhibitor, a histone deacetylase (HDAC) inhibitor, an HSP90 inhibitor, a VEGFR inhibitor, an AKT inhibitor, chemotherapy, or any combination thereof.

In some embodiments, a compound of Formula (A), (B), or (C), or a pharmaceutically acceptable salt thereof, is used in combination with hormone blocking therapy, chemotherapy, radiation therapy, monoclonal antibodies, or combinations thereof.

Hormone blocking therapy includes the use of agents that block the production of estrogens or block the estrogen receptors. In some embodiments, hormone blocking therapy includes the use of estrogen receptor modulators and/aromatase inhibitors. Estrogen receptor modulators include triphenylethylene derivatives (e.g. tamoxifen, toremifene, droloxifene, 3-hydroxytamoxifen, idoxifene, TAT-59 (a phosphorylated derivative of 4-hydroxytamoxifen) and GW5638 (a carboxylic acid derivative of tamoxifen)); non-steroidal estrogen receptor modulators (e.g. raloxifene, LY353381 (SERM3) and LY357489); steroidal estrogen receptor modulators (e.g. ICI-182,780). Aromatase inhibitors include steroidal aromatase inhibitors and non-steroidal aromatase inhibitors. Steroidal aromatase inhibitors include, but are not limited to, such exemestane. Non-steroidal aromatase inhibitors include, but are not limited to, as anastrozole, and letrozole.

Chemotherapy includes the use of anti-cancer agents.

Monoclonal antibodies include, but are not limited to, trastuzumab (Herceptin).

In some embodiments, a compound of Formula (A), (B), or (C), or a pharmaceutically acceptable salt thereof, is used in combination with at least one additional therapeutic agent selected from: abiraterone; abarelix; adriamycin; aactinomycin; acivicin; aclarubicin; acodazole hydrochloride; acronine; adozelesin; aldesleukin; alemtuzumab; allopurinol; alitretinoin; altretamine; ambomycin; ametantrone acetate; aminoglutethimide; aminolevulinic acid; amifostine; amsacrine; anastrozole; anthramycin; aprepitant; arsenic trioxide; asparaginase; asperlin; azacitidine; azetepa; azotomycin; batimastat; bendamustine hydrochloride; benzodepa; bevacizumab; bexarotene; bicalutamide; bisantrene hydrochloride; bisnafide dimesylate; bizelesin; bleomycin; bleomycin sulfate; bortezomib; brequinar sodium; bropirimine; busulfan; cactinomycin; calusterone; caracemide; carbetimer; carboplatin; carmustine; carubicin hydrochloride; carzelesin; capecitabine; cedefingol; cetuximab; chlorambucil; cirolemycin; cisplatin; cladribine; clofarabine; crisnatol mesylate; cyclophosphamide; cytarabine; dacarbazine; dasatinib; daunorubicin hydrochloride; dactinomycin; darbepoetin alfa; decitabine; degarelix; denileukin diftitox; dexormaplatin; dexrazoxane hydrochloride; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; eltrombopag olamine; enloplatin; enpromate; epipropidine; epirubicin hydrochloride; epoetin alfa; erbulozole; erlotinib hydrochloride; esorubicin hydrochloride; estramustine; estramustine phosphate sodium; etanidazole; etoposide; etoposide phosphate; etoprine; everolimus; exemestane; fadrozole hydrochloride; fazarabine; fenretinide; filgrastim; floxuridine; fludarabine phosphate; fluorouracil; flurocitabine; fosquidone; fostriecin sodium; fulvestrant; gefitinib; gemcitabine; gemcitabine hydrochloride; gemcitabine-cisplatin; gemtuzumab ozogamicin; goserelin acetate; histrelin acetate; hydroxyurea; idarubicin hydrochloride; ifosfamide; iimofosine; ibritumomab tiuxetan; idarubicin; ifosfamide; imatinib mesylate; imiquimod; interleukin Il (including recombinant interleukin II, or r1L2), interferon alfa-2a; interferon alfa-2b; interferon alfa-n1; interferon alfa-n3; interferon beta-1 a; interferon gamma-1 b; iproplatin; irinotecan hydrochloride; ixabepilone; lanreotide acetate; lapatinib; lenalidomide; letrozole; leuprolide acetate; leucovorin calcium; leuprolide acetate; levamisole; liposomal cytarabine; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; methoxsalen; metoprine; meturedepa; mitindomide; mitocarcin; mitocromin; mitogillin; mitomalcin; mitomycin C; mitosper; mitotane; mitoxantrone hydrochloride; mycophenolic acid; nandrolone phenpropionate; nelarabine; nilotinib; nocodazoie; nofetumomab; nogalamycin; ofatumumab; oprelvekin; ormaplatin; oxaliplatin; oxisuran; paclitaxel; palifermin; palonosetron hydrochloride; pamidronate; pegfilgrastim; pemetrexed disodium; pentostatin; panitumumab; pazopanib hydrochloride; pemetrexed disodium; plerixafor; pralatrexate; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; quinacrine; raloxifene hydrochloride; rasburicase; recombinant HPV bivalent vaccine; recombinant HPV quadrivalent vaccine; riboprine; rogletimide; rituximab; romidepsin; romiplostim; safingol; safingol hydrochloride; sargramostim; semustine; simtrazene; sipuleucel-T; sorafenib; sparfosate sodium; sparsomycin; spirogermanium hydrochloride; spiromustine; spiroplatin; streptonigrin; streptozocin; sulofenur; sunitinib malate; talisomycin; tamoxifen citrate; tecogalan sodium; tegafur; teloxantrone hydrochloride; temozolomide; temoporfin; temsirolimus; teniposide; teroxirone; testolactone; thalidomide; thiamiprine; thioguanine; thiotepa; tiazofurin; tirapazamine; topotecan hydrochloride; toremifene; tositumomab and I 131 Iodine tositumomab; trastuzumab; trestolone acetate; tretinoin; triciribine phosphate; trimetrexate; trimetrexate glucuronate; triptorelin; tubulozole hydrochloride; uracil mustard; uredepa; valrubicin; vapreotide; verteporfin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorinostat; vorozole; zeniplatin; zinostatin; zoledronic acid; or zorubicin hydrochloride.

In some embodiments, the compound of Formula (A), (B), or (C), or a pharmaceutically acceptable salt thereof, is administered or formulated in combination with one or more chemotherapeutic agent selected from, by way of example only, alemtuzumab, arsenic trioxide, asparaginase (pegylated or non-), bevacizumab, temsirolimus, cetuximab, platinum-based compounds such as cisplatin, cladribine, daunorubicin/doxorubicin/idarubicin, irinotecan, fludarabine, 5-fluorouracil, gemtuzumab, methotrexate, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, or dronabinol.

In one aspect, the compound of Formula (A), (B), or (C), or a pharmaceutically acceptable salt thereof, is administered or formulated in combination with one or more anti-cancer agents. In some embodiments, one or more of the anti-cancer agents are proapoptotic agents. Examples of anti-cancer agents include, but are not limited to, any of the following: gossypol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib, geldanamycin, 17-N-Allylamino-17-Demethoxygeldanamycin (17-AAG), flavopiridol, LY294002, bortezomib, trastuzumab, BAY 11-7082, PKC412, or PD184352, paclitaxel, and analogs of paclitaxel. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with the compounds of Formula (A), (B), or (C), or a pharmaceutically acceptable salt thereof, include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Further examples of anti-cancer agents for use in combination with the compounds of Formula (A), (B), or (C), or a pharmaceutically acceptable salt thereof, include aromatase inhibitors. Aromatase inhibitors include steroidal aromatase inhibitors and non-steroidal aromatase inhibitors. Steroidal aromatase inhibitors include, but are not limited to, exemestane. Non-steroidal aromatase inhibitors include, but are not limited to, anastrozole, and letrozole. In some embodiments, the aromatase inhibitor is anastrozole, letrozole or exemestane.

In some embodiments, a compound of Formula (A), (B), or (C), or a pharmaceutically acceptable salt thereof, is administered in combination with a CDK 4/6 inhibitor. In some embodiments, the CDK 4/6 inhibitor is LEE011 or LY283519.

In some embodiments, a compound of Formula (A), (B), or (C), or a pharmaceutically acceptable salt thereof, is administered in combination with a phosphoinositide 3-kinase (PI3K)/mTOR pathway inhibitor. In some embodiments, the a phosphoinositide 3-kinase (PI3K)/mTOR pathway inhibitor is everolimus, temsirolimus, BEZ235, BYL719, GDC-0032, BKM120, BGT226, GDC-0068, GDC-0980, GDC-0941, INK128 (MLN0128), INK1117, MK-2206, OSI-027, CC-223, AZD8055, SAR245408, SAR245409, PF04691502, WYE125132, GSK2126458, GSK-2636771, BAY806946, PF-05212384, SF1126, PX866, AMG319, ZSTK474, Cal101, PWT33597, CU-906, or CUDC-907.

In some embodiments, a compound of Formula (A), (B), or (C), or a pharmaceutically acceptable salt thereof, is administered in combination with a histone deacetylase inhibitor (HDAC). In some embodiments, the HDAC inhibitor is entinostat or mocetinostat.

In some embodiments, a compound of Formula (A), (B), or (C), or a pharmaceutically acceptable salt thereof, is administered in combination with a HER-2 inhibitor. In some embodiments, the HER-2 inhibitor is trastuzumab, pertuzumab or TDM-1.

In some embodiments, a compound of Formula (A), (B), or (C), or a pharmaceutically acceptable salt thereof, is administered in combination with an epidermal growth factor receptor (EGFR) inhibitor. In some embodiments, the EGFR inhibitor is lapatinib, gefitinib, erlotinib, cetuximab, canertinib, panitumumab, nimotuzumab, OSI-632, vandetanib, afatinib, MP-412, AEE-788, neratinib, XL-647, dacomitinib, AZD-8931, CUDC-101, AP-26113 or CO-1686.

In some embodiments, a compound of Formula (A), (B), or (C), or a pharmaceutically acceptable salt thereof, is administered in combination with an anti-angiogenesis agent. In some embodiments, the anti-angiogenesis agent is a VEGFR inhibitor. In some embodiments, the anti-angiogenesis agent is a multi-kinase targeting agent. In some embodiments, the anti-angiogenesis agent is bevacizumab, ABR-215050 (tasquinimod), CHIR-258 (dovitinib), EXEL-7647, OSI-930, BIBF-1120, BAY-73-4506, BMS-582664 (brivanib), RO-4929097, JNJ-26483327, AZD-2171 (cediranib), sorafenib, aflibercept, enzastaurin, AG-013736 (axitinib), GSK-786034 (pazopanib), AP-23573, or sunitinib.

In some embodiments, a compound of Formula (A), (B), or (C), or a pharmaceutically acceptable salt thereof, is administered in combination with an anti-PD-1 agent. In some embodiments, the anti-PD-1 agent is MK-3475, Nivolumab, MPDL3280A, or MEDI4736.

In some embodiments, a compound of Formula (A), (B), or (C), or a pharmaceutically acceptable salt thereof, is administered in combination with an AKT inhibitor. In some embodiments, the AKT inhibitor is GDC-0068, MK-2206, AT7867, GSK2110183, GSK2141795 named as N-[(1S)-2-amino-1-[(3,4-difluorophenyl)methyl]ethyl]-5-chloro-4-(4-chloro-1-methyl-1H-pyrazol-5-yl)-2-furancarboxamide (WO 2008/098104), GSK690693, or AZD5363 named as (S)-4-amino-N-[1-(4-chlorophenyl)-3-hydroxypropyl]-1-(7H-pyrrolo[2,3-d]pyrimidin 4-yl)piperidine-4-carboxamide (Davies B R, et al (2012) Mol Canc Ther, 11:873-887).

In some embodiments, a compound of Formula (A), (B), or (C), or a pharmaceutically acceptable salt thereof, is administered in combination with doxorubicin, cyclophosphamide, capecitabine, vinorelbine, paclitaxel, doxetaxel, or cisplatin.

Yet other anticancer agents for use in combination with the compounds of Formula (A), (B), or (C), or a pharmaceutically acceptable salt thereof, include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, ete.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products for use in combination with the compounds of Formula (A), (B), or (C), or a pharmaceutically acceptable salt thereof, include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents for use in combination with the compounds of Formula (A), (B), or (C), or a pharmaceutically acceptable salt thereof, include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.).

In some embodiments, compounds of Formula (A), (B), or (C), or a pharmaceutically acceptable salt thereof, are used to treat cancer in combination with: a second antiestrogen (e.g., tamoxifen), an antiandrogen (e.g., bicalutamide, flutamide), a gonadotropin releasing hormone analog (e.g., leuprolide).

Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules include without limitation the following marketed drugs and drugs in development: Erbulozole, Dolastatin 10, Mivobulin isethionate, Vincristine, NSC-639829, Discodermolide, ABT-751, Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride, Epothilones (such as Epothilone A, Epothilone B, Epothilone C, Epothilone D, Epothilone E, Epothilone F, Epothilone B N-oxide, Epothilone A N-oxide, 16-aza-epothilone B, 21-aminoepothilone B, 21-hydroxyepothilone D, 26-fluoroepothilone, Auristatin PE, Soblidotin, Vincristine sulfate, Cryptophycin 52, Vitilevuamide, Tubulysin A, Canadensol, Centaureidin, Oncocidin A1 Fijianolide B, Laulimalide, Narcosine, Nascapine, Hemiasterlin, Vanadocene acetylacetonate, Indanocine Eleutherobins (such as Desmethyleleutherobin, Desaetyleleutherobin, lsoeleutherobin A, and Z-Eleutherobin), Caribaeoside, Caribaeolin, Halichondrin B, Diazonamide A, Taccalonolide A, Diozostatin, (−)-Phenylahistin, Myoseverin B, Resverastatin phosphate sodium.

In one aspect, a compound of Formula (A), (B), or (C), or a pharmaceutically acceptable salt thereof, is co-administered with thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048.

In some embodiments, a compound of Formula (A), (B), or (C), or a pharmaceutically acceptable salt thereof, is used in combination with anti-emetic agents to treat nausea or emesis, which may result from the use of a compound of Formula (A), (B), or (C), or a pharmaceutically acceptable salt thereof, anti-cancer agent(s) and/or radiation therapy.

Anti-emetic agents include, but are not limited to: neurokinin-1 receptor antagonists, 5HT3 receptor antagonists (such as ondansetron, granisetron, tropisetron, palonosetron, and zatisetron), GABA_{B} receptor agonists (such as baclofen), corticosteroids (such as dexamethasone, prednisone, prednisolone, or others), dopamine antagonists (such as, but not limited to, domperidone, droperidol, haloperidol, chlorpromazine, promethazine, prochlorperazine, metoclopramide), antihistamines (H1 histamine receptor antagonists, such as but not limited to, cyclizine, diphenhydramine, dimenhydrinate, meclizine, promethazine, hydroxyzine), cannabinoids (such as but not limited to, cannabis, marinol, dronabinol), and others (such as, but not limited to, trimethobenzamide; ginger, emetrol, propofol).

In some embodiments, a compound of Formula (A), (B), or (C), or a pharmaceutically acceptable salt thereof, is used in combination with an agent useful in the treatment of anemia. Such an anemia treatment agent is, for example, a continuous eythropoiesis receptor activator (such as epoetin-α).

In some embodiments, a compound of Formula (A), (B), or (C), or a pharmaceutically acceptable salt thereof, is used in combination with an agent useful in the treatment of neutropenia. Examples of agents useful in the treatment of neutropenia include, but are not limited to, a hematopoietic growth factor which regulates the production and function of neutrophils such as a human granulocyte colony stimulating factor, (G-CSF). Examples of a G-CSF include filgrastim.

In some embodiments, a compound of Formula (A), (B), or (C), or a pharmaceutically acceptable salt thereof, is administered with corticosteroids. Corticosteroids, include, but are not limited to: betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

In one embodiment, a compound of Formula (A), (B), or (C), or a pharmaceutically acceptable salt thereof, is administered to a mammal in combination with a non-steroidal anti-inflammatory drug (NSAID). NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, lumiracoxib, CS-502, JTE-522, L-745,337 and NS398).

In some embodiments, a compound of Formula (A), (B), or (C), or a pharmaceutically acceptable salt thereof, is coadministered with an analgesic.

In some embodiments, a compound of Formula (A), (B), or (C), or a pharmaceutically acceptable salt thereof, is used in combination with radiation therapy (or radiotherapy). Radiation therapy is the treatment of cancer and other diseases with ionizing radiation. Radiation therapy can be used to treat localized solid tumors, such as cancers of the skin, tongue, larynx, brain, breast, prostate, colon, uterus and/or cervix. It can also be used to treat leukemia and lymphoma (cancers of the blood-forming cells and lymphatic system, respectively).

A technique for delivering radiation to cancer cells is to place radioactive implants directly in a tumor or body cavity. This is called internal radiotherapy (brachytherapy, interstitial irradiation, and intracavitary irradiation are types of internal radiotherapy.) Using internal radiotherapy, the radiation dose is concentrated in a small area, and the patient stays in the hospital for a few days. Internal radiotherapy is frequently used for cancers of the tongue, uterus, prostate, colon, and cervix.

The term “radiotherapy” or “ionizing radiation” include all forms of radiation, including but not limited to α, β, and γ radiation and ultraviolet light.

In some embodiments, a compound of Formula (A), (B), or (C), or a pharmaceutically acceptable salt thereof, is used in the treatment of breast cancer in combination with at least one additional treatment option for the breast cancer. In some embodiments, a compound of Formula (A), (B), or (C), or a pharmaceutically acceptable salt thereof, is used either alone or in combination with other agents used to treat breast cancer, including but not limited to aromatase inhibitors, anthracylines, platins, nitrogen mustard, alkylating agents, taxanes, nucleoside analogs, a phosphoinositide 3-kinase (PI3K)/mTOR pathway inhibitor, CDK 4/6 inhibitors, HER-2 inhibitors, EGFR inhibitors, PD-1 inhibitors, poly ADP-ribose polymerase (PARP) inhibitors, histone deacetylase (HDAC) inhibitors, and HSP90 inhibitors. Illustrative agents used to treat breast cancer, include, but are not limited to, fulvestrant, tamoxifen, anastrozole, letrozole, exemestane, GDC0032, goserelin, leuprolide, raloxifene, toremifene, megestrol acetate, bazedoxifene, cisplatin, carboplatin, capecitabine, cyclophosphamide, docetaxel, doxorubicin, epirubicin, eribulin, filgrastim, fluorouracil, gemcitabine, ixabepilone, LEE011, LY2835219, mitoxantrone, methotrexate, paclitaxel, pamidronate, vinorelbine, pegfilgrastim, pertuzumab, trastuzumab, lapatinib, everolimus, bevacizumab, temsirolimus and combinations thereof, as well as others described herein. Additional non-limiting exemplary agents for the treatment of breast cancer are provided elsewhere herein. In some embodiments, a compound of Formula (A), (B), or (C), or a pharmaceutically acceptable salt thereof, is used either alone or in combination with breast cancer surgery. In some embodiments, breast cancer surgery comprises lumpectomy, mastectomy, sentinel node biopsy, or axillary node dissection. In some embodiments, a compound of Formula (A), (B), or (C), or a pharmaceutically acceptable salt thereof, is used either alone or in combination with radiation therapy. In some embodiments, radiation comprises external beam radiation or brachytherapy. In some embodiments, a compound of Formula (A), (B), or (C), or a pharmaceutically acceptable salt thereof, is used either alone or in combination with hormone therapy (i.e. hormone blocking therapy). In some embodiments, hormone therapy comprises the use of a selective estrogen receptor modulator (e.g. tamoxifen), aromatase inhibitor, or fulvestrant. In some embodiments, a compound of Formula (A), (B), or (C), or a pharmaceutically acceptable salt thereof, is used either alone or in combination with surgery to remove the ovaries or medications to stop the ovaries from making estrogen. In some embodiments, a compound of Formula (A), (B), or (C), or a pharmaceutically acceptable salt thereof, is used either alone or in combination with trastuzumab, lapatinib, or bevacizumab. In some embodiments, a compound of Formula (A), (B), or (C), or a pharmaceutically acceptable salt thereof, is used either alone or in combination with bone-building drugs to prevent breast cancer recurrence (e.g. zoledronic acid (Reclast, Zometa)).

GDC-0032, also known as taselisib (CAS Reg. No. 1282512-48-4, Genentech Inc.,), is a phosphoinositide 3-kinase (PI3K)/mTOR pathway inhibitor. GDC-0032 is named as 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide, and has the structure:

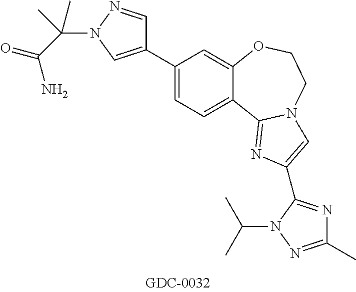 [SMILES: Cc1nc(-c2cn3c(n2)-c2ccc(-c4cnn(C(C)(C)C(N)=O)c4)cc2OCC3)n(C(C)C)n1]

including stereoisomers, geometric isomers, tautomers, and pharmaceutically acceptable salts thereof. GDC-0032 can be prepared and characterized as described in WO 2011/036280, U.S. Pat. No. 8,242,104, and U.S. Pat. No. 8,343,955.

GDC-0941, also known as pictilisib or pictrelisib, (CAS Reg. No. 957054-30-7, Genentech Inc., Roche, RG-7321) is a potent multitargeted class I (pan) inhibitor of PI3K isoforms. GDC-0941 is currently in phase II clinical trials for the treatment of advanced solid tumors. GDC-0941 is named as 4-(2-(1H-indazol-4-yl)-6-((4-(methylsulfonyl)piperazin-1-yl)methyl)thieno[3,2-d]pyrimidin-4-yl)morpholine (U.S. Pat. No. 7,781,433; U.S. Pat. No. 7,750,002; Folkes et al (2008) Jour. of Med. Chem. 51(18):5522-5532), and has the structure:

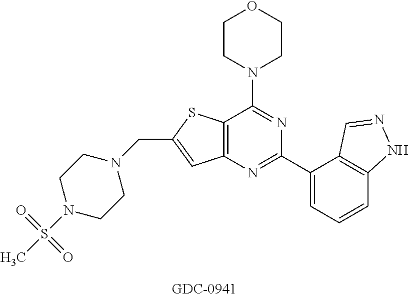 [SMILES: CS(=O)(=O)N1CCN(Cc2cc3nc(-c4cccc5[nH]ncc45)nc(N4CCOCC4)c3s2)CC1]

including stereoisomers, geometric isomers, tautomers, and pharmaceutically acceptable salts thereof.

GDC-0068, also known as ipatasertib (CAS Reg. No. 1001264-89-6, Genentech Inc., Roche, RG-7440) is a highly selective, pan-Akt inhibitor targeting Akt1/2/3 in clinical trials for the potential oral treatment of solid tumors. GDC-0068 is named as (S)-2-(4-chlorophenyl)-1-(4-((5R,7R)-7-hydroxy-5-methyl-6,7-dihydro-5H-cyclopenta[d]pyrimidin-4-yl)piperazin-1-yl)-3-(isopropylamino)propan-1-one (U.S. Pat. No. 8,853,199), and has the structure:

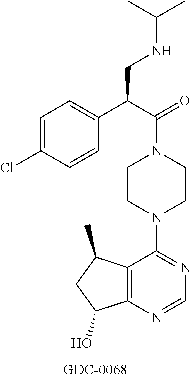 [SMILES: CC(C)NC[C@@H](C(=O)N1CCN(c2ncnc3c2[C@H](C)C[C@H]3O)CC1)c1ccc(Cl)cc1]

including stereoisomers, geometric isomers, tautomers, and pharmaceutically acceptable salts thereof.

### Estrogen Receptor Modulator and Chemotherapeutic Combination—In Vitro Activity

The cytotoxic or cytostatic activity of combinations of estrogen receptor modulator (ERM) compounds, including but not limited to those in Tables 1, 2, and 3, and exemplary chemotherapeutic agents is measured by: establishing a proliferating mammalian tumor cell line in a cell culture medium, adding a test compound, culturing the cells for a period from about 6 hours to about 5 days; and measuring cell viability (Example 3). Cell-based in vitro assays were used to measure viability, i.e. proliferation (IC₅₀), cytotoxicity (EC₅₀), and induction of apoptosis (caspase activation).

The in vitro potency of the combinations of ERM compounds with chemotherapeutic agents is measured by the cell proliferation assay of Example 7; the CellTiter-Glo® Luminescent Cell Viability Assay, commercially available from Promega Corp., Madison, Wis. This homogeneous assay method is based on the recombinant expression of Coleoptera luciferase (U.S. Pat. No. 5,583,024; U.S. Pat. No. 5,674,713; U.S. Pat. No. 5,700,670) and determines the number of viable cells in culture based on quantitation of the ATP present, an indicator of metabolically active cells (Crouch et al (1993) J. Immunol. Meth. 160:81-88; U.S. Pat. No. 6,602,677). The CellTiter-Glo® Assay was conducted in 96 or 384 well format, making it amenable to automated high-throughput screening (HTS) (Cree et al (1995) AntiCancer Drugs 6:398-404). The homogeneous assay procedure involves adding the single reagent (CellTiterGlo® Reagent) directly to cells cultured in serum-supplemented medium. Cell washing, removal of medium and multiple pipetting steps are not required. The system detects as few as 15 cells/well in a 384-well format in 10 minutes after adding reagent and mixing.

The anti-proliferative effects of combinations of ERM compounds and chemotherapeutic agents are measured by the CellTiter-Glo® Assay (Example 3) against tumor cell lines. EC₅₀ values are established for the tested compounds and combinations. The range of in vitro cell potency activities may be about 100 nM to about 10 μM.

The individual measured EC50 values of ERM compounds and of the chemotherapeutic agent against the particular cell are compared to the combination EC50 value. The combination index (CI) score is calculated by the Chou and Talalay method (Chou, T. and Talalay, P. (1984) Adv. Enzyme Regul. 22:27-55). A CI less than about 0.7 indicates synergy. A CI between 0.8 and 1.2 indicates additivity. A CI greater than 1.2 indicates antagonism. The strength of synergy is assessed according to Chou and Talalay. Certain therapeutic combinations show the surprising and unexpected property of synergy in the in vitro cell proliferation assays with tumor type cell lines including breast cancer cells. Other combinations show no synergy; and only show mere additivity or antagonism. Certain combinations are synergistic with one or more tumor types, but not others. The synergy demonstrated in the in vitro cell proliferation assays provides a basis to expect a corresponding synergy in treating cancer in human patients.

### Estrogen Receptor Modulator and Chemotherapeutic Combinations—In Vivo Tumor Xenograft Activity

The efficacy of the combinations of ERM compounds and various chemotherapeutic agents was measured in vivo by implanting allografts or xenografts of cancer cells in rodents and treating the tumor-bearing animals with the drug combinations. Results are dependent on the cell line, the presence or absence of certain mutations in the tumor cells, the sequence of administration of ERM compounds and chemotherapeutic agent, dosing regimen, and other factors. Subject mice were treated with drug(s) or control (Vehicle) and monitored over several weeks or more to measure the time to tumor doubling, log cell kill, and tumor inhibition (Example 4). FIGS. 23-36 show plots of tumor volume change over time after treatment of tumor-bearing mice treated with combinations of ERM compounds and various chemotherapeutic agents according to the protocol of Example 8.

FIG. 1 shows the fitted tumor volume change over 42 days in cohorts of 8 immunocompromised mice bearing HCI-003 breast tumor (BC PDX model) xenografts harboring ESR1 WT (wild type) and PIK3CA H1047R (PI3Kα) mutation, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 1-3 from Table 1 at 10 mg/kg, and the combination of ERM 1-3 and GDC-0032. Vehicle (+) is solvent/buffer with ethynyl estradiol (0.1 mg/kg). Vehicle (−) is solvent/buffer without ethynyl estradiol. The HCI-003 breast tumor model is a PI3K homozygous mutant and very sensitive to PI3K inhibition. ERM 1-3 enhances the efficacy of GDC-0032 by increasing tumor regressions in HCI-003.

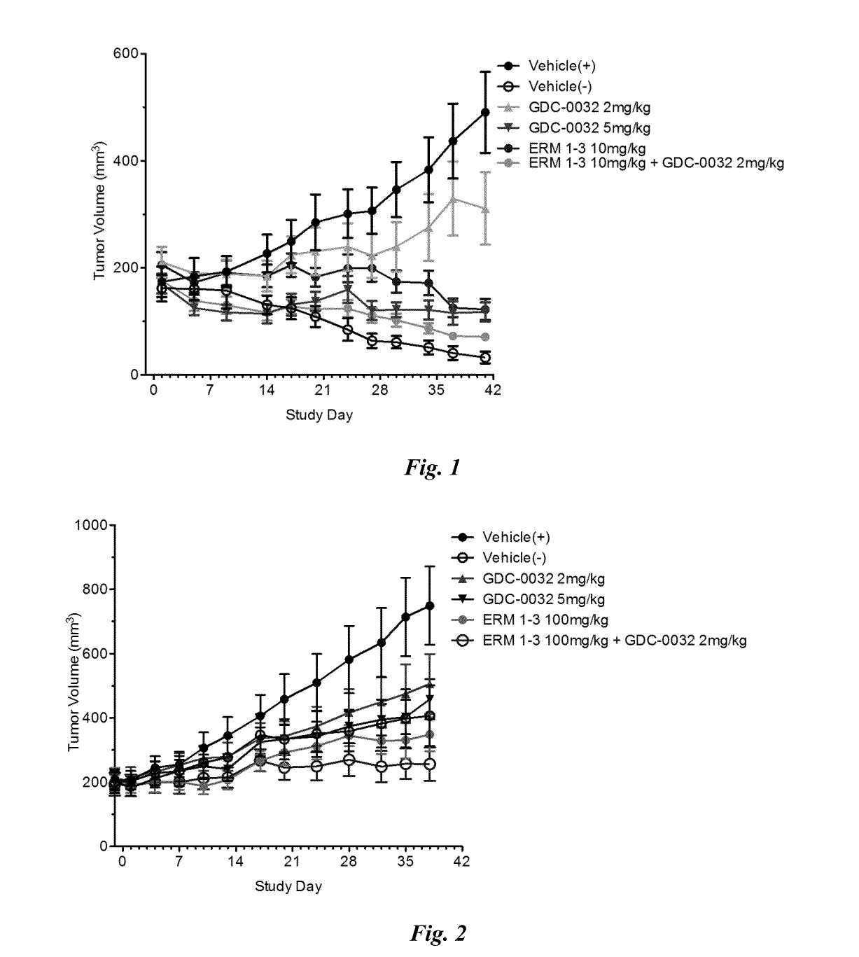

FIG. 2 shows the fitted tumor volume change over 38 days in cohorts of 8 immunocompromised mice bearing HCI-005 breast tumor (BC PDX model) xenografts harboring ESR1 L536P mutant, HER2+, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 1-3 from Table 1 at 100 mg/kg, and the combination of ERM 1-3 and GDC-0032. The HCI-005 breast tumor model is ESR1 mutant and HER2+. ERM 1-3 enhances the efficacy of GDC-0032 by increasing tumor regressions in HCI-005.

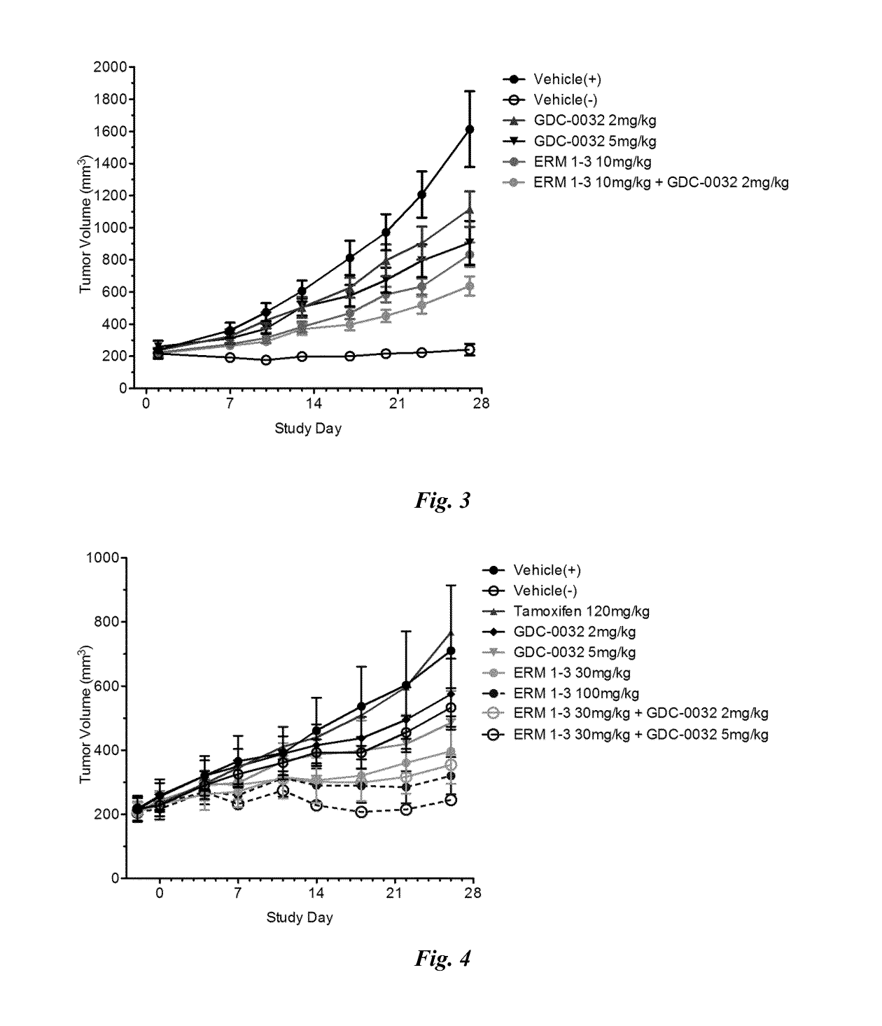

FIG. 3 shows the fitted tumor volume change over 27 days in cohorts of 8 immunocompromised mice bearing HCI-011 breast tumor (BC PDX model) xenografts harboring ESR1 WT (wild type) and PIK3CA E545K (PI3Kα) mutation, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 1-3 from Table 1 at 10 mg/kg, and the combination of ERM 1-3 and GDC-0032. The HCI-011 breast tumor model is PI3K mutant E545K. ERM 1-3 enhances the efficacy of GDC-0032 by increasing tumor regressions in HCI-011.

FIG. 4 shows the fitted tumor volume change over 26 days in cohorts of 8 or 9 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, PI3K inhibitor GDC-0032 at 2 mg/kg and 5 mg/kg, estrogen receptor modulator (ERM) 1-3 from Table 1 at 30 mg/kg and 100 mg/kg, and combinations of ERM 1-3 (30 mg/kg) and GDC-0032 (2 and 5 mg/kg). ERM 1-3 enhances the efficacy of GDC-0032 by increasing tumor regressions in TamR1. The combination of ERM 1-3 and GDC-0032 is not more efficacious than ERM 1-3 single agent anti-tumor activity in TamR1.

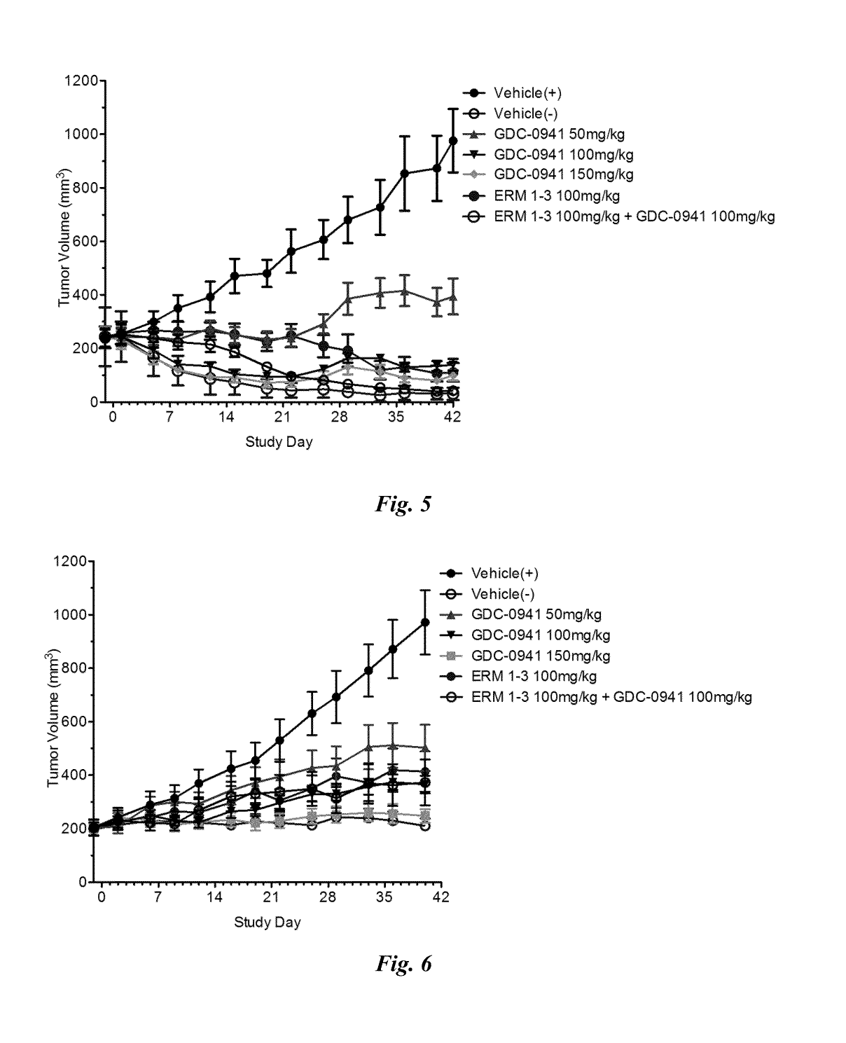

FIG. 5 shows the fitted tumor volume change over 42 days in cohorts of 7 immunocompromised mice bearing HCI-003 breast tumor (BC PDX model) xenografts harboring ESR1 WT (wild type) and PIK3CA H1047R (PI3Kα) mutation, dosed daily by 100 microliter (μl) PO (oral) administration with Vehicle (+), Vehicle (−), pan-PI3K inhibitor GDC-0941 at 50, 100 and 150 mg/kg, estrogen receptor modulator (ERM) 1-3 from Table 1 at 100 mg/kg, and the combination of ERM 1-3 and GDC-0941. ERM 1-3 enhances the efficacy of GDC-0941 by increasing tumor regressions in HCI-003.

FIG. 6 shows the fitted tumor volume change over 40 days in cohorts of 8 immunocompromised mice bearing HCI-005 breast tumor (BC PDX model) xenografts harboring ESR1 L536P mutant, and HER2+, dosed daily by 100 microliter (μl) PO (oral) administration with Vehicle (+), Vehicle (−), pan-PI3K inhibitor GDC-0941 at 50, 100 and 150 mg/kg, estrogen receptor modulator (ERM) 1-3 from Table 1 at 100 mg/kg, and the combination of ERM 1-3 and GDC-0941. The HCI-005 breast tumor model is ESR1 mutant and HER2+. ERM 1-3 enhances the efficacy of 100 mg/kg GDC-0941 by increasing tumor regressions in HCI-005.

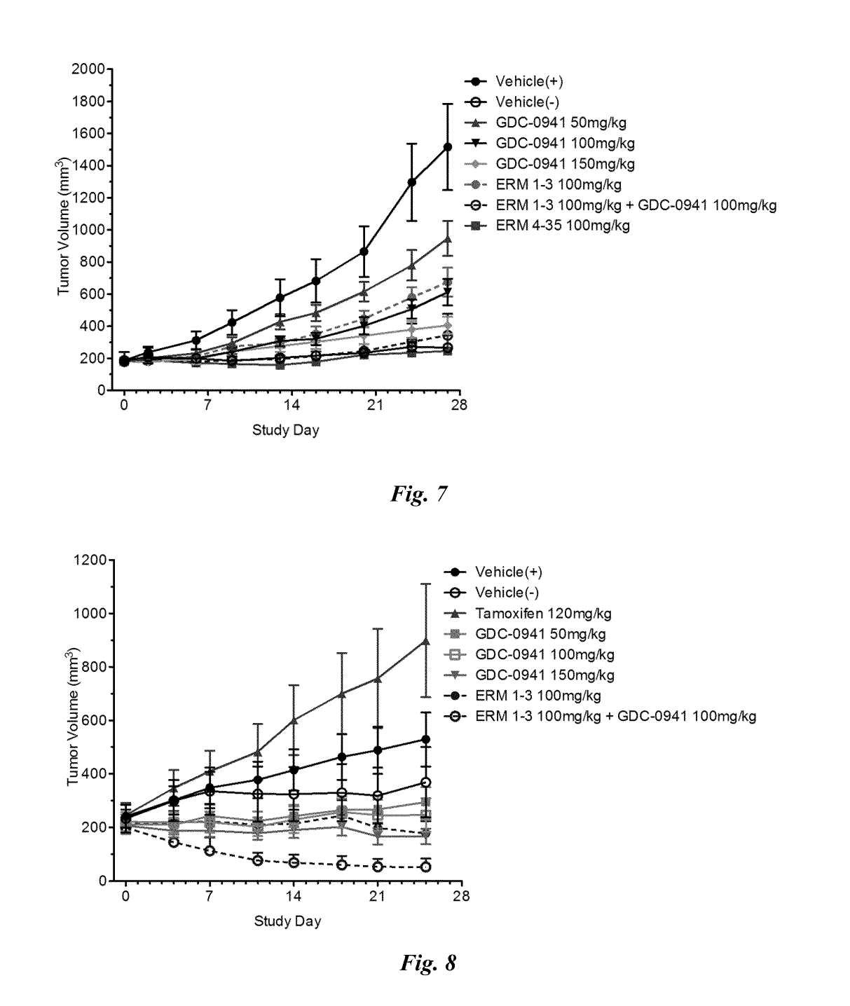

FIG. 7 shows the fitted tumor volume change over 27 days in cohorts of 8 immunocompromised mice bearing HCI-011 breast tumor (BC PDX model) xenografts harboring ESR1 WT (wild type) and PIK3CA E545K (PI3Kα) mutation, dosed daily by 100 microliter (μl) PO (oral) administration with Vehicle (+), Vehicle (−), pan-PI3K inhibitor GDC-0941 at 50, 100 and 150 mg/kg, estrogen receptor modulator (ERM) 1-3 from Table 1 at 100 mg/kg, the combination of ERM 1-3 and GDC-0941, and ERM 4-35 from Table 3. The HCI-011 breast tumor model is PI3K mutant E545K. ERM 1-3 enhances the efficacy of GDC-0941 by increasing tumor regressions in HCI-011.

FIG. 8 shows the fitted tumor volume change over 25 days in cohorts of 8 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, pan-PI3K inhibitor GDC-0941 at 50, 100 and 150 mg/kg, estrogen receptor modulator (ERM) 1-3 from Table 1 at 100 mg/kg, and the combination of ERM 1-3 (30 mg/kg) and GDC-0941. ERM 1-3 enhances the efficacy of 100 mg/kg GDC-0032 by increasing tumor regressions in TamR1.

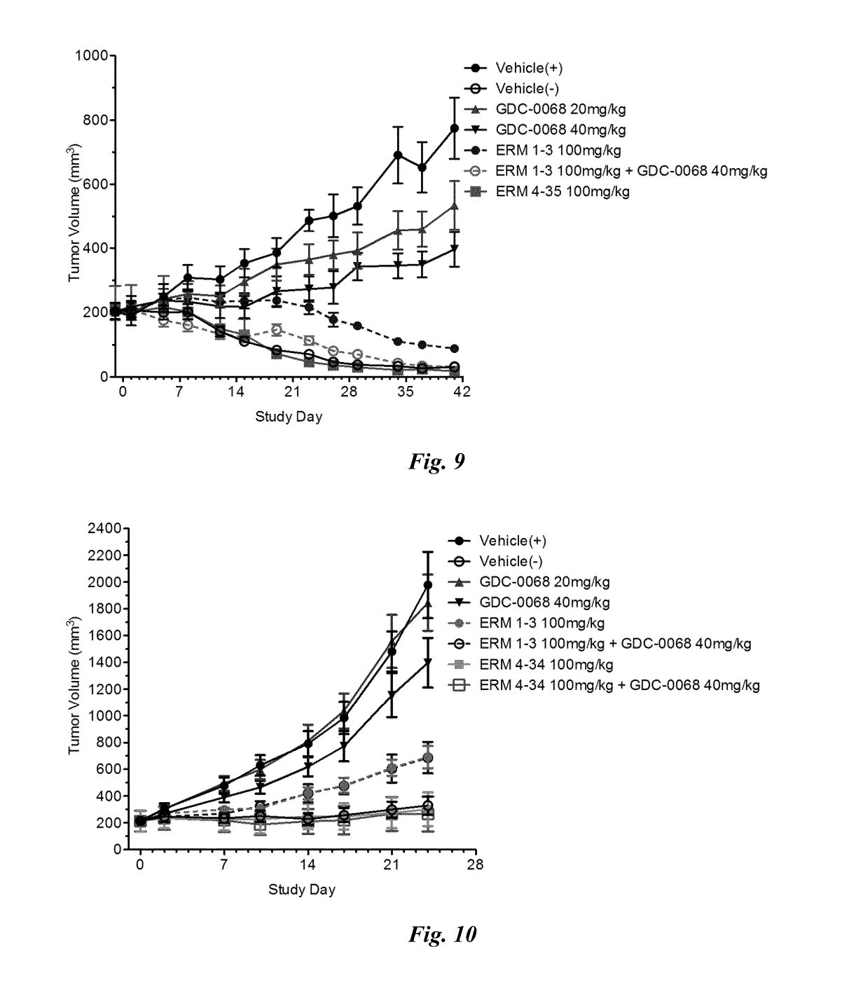

FIG. 9 shows the fitted tumor volume change over 41 days in cohorts of 7 immunocompromised mice bearing HCI-003 breast tumor (BC PDX model) xenografts harboring ESR1 WT (wild type) and PIK3CA H1047R (PI3Kα) mutation, dosed daily by 100 microliter (μl) PO (oral) administration with Vehicle (+), Vehicle (−), AKT inhibitor GDC-0068 at 20 and 40 mg/kg, estrogen receptor modulator (ERM) 1-3 from Table 1 at 100 mg/kg, the combination of ERM 1-3 and GDC-0068, and ERM 4-35 from Table 3. ERM 1-3 at 100 mg/kg enhances the efficacy of 40 mg/kg GDC-0068 by increasing tumor regressions in HCI-003.

FIG. 10 shows the fitted tumor volume change over 23 days in cohorts of 10 immunocompromised mice bearing HCI-011 breast tumor (BC PDX model) xenografts harboring ESR1 WT (wild type) and PIK3CA E545K (PI3Kα) mutation, dosed daily by 100 microliter (μl) PO (oral) administration with Vehicle (+), Vehicle (−), AKT inhibitor GDC-0068 at 20 and 40 mg/kg, estrogen receptor modulator (ERM) 1-3 from Table 1 at 100 mg/kg, the combination of ERM 1-3 and GDC-0941, estrogen receptor modulator (ERM) 4-34 from Table 3 at 100 mg/kg, and the combination of ERM 4-34 and GDC-0068. The combinations of ERM 1-3 and GDC-0068, and ERM 4-34 and GDC-0068 are not more efficacious than each single agent anti-tumor activity in HCI-011.

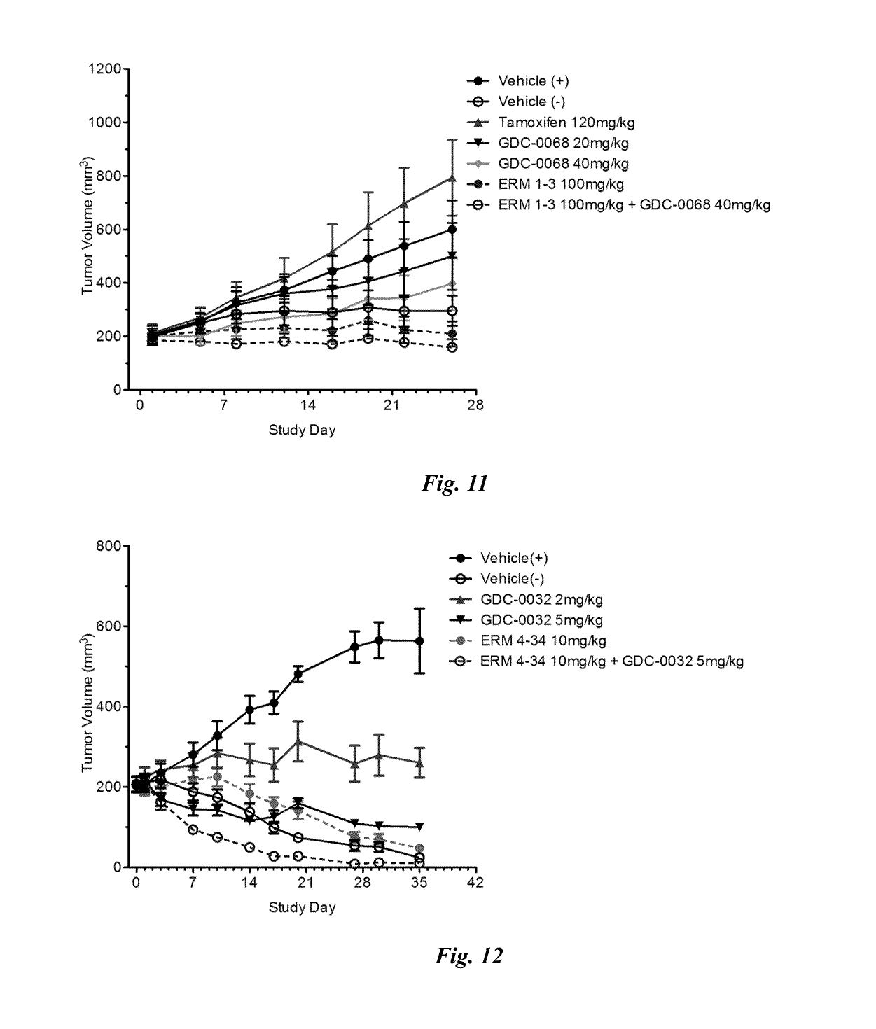

FIG. 11 shows the fitted tumor volume change over 26 days in cohorts of 9 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, AKT inhibitor GDC-0068 at 20 and 40 mg/kg, estrogen receptor modulator (ERM) 1-3 from Table 1 at 100 mg/kg, and the combination of ERM 1-3 (30 mg/kg) and GDC-0068.

FIG. 12 shows the fitted tumor volume change over 35 days in cohorts of 8 immunocompromised mice bearing HCI-003 breast tumor (BC PDX model) xenografts harboring ESR1 WT (wild type) and PIK3CA H1047R (PI3Kα) mutation, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 4-34 from Table 3 at 10 mg/kg, and the combination of ERM 4-34 and GDC-0032. The HCI-003 breast tumor model is a PI3K homozygous mutant and very sensitive to PI3K inhibition. ERM 4-34 enhances the efficacy of GDC-0032 by increasing tumor regressions.

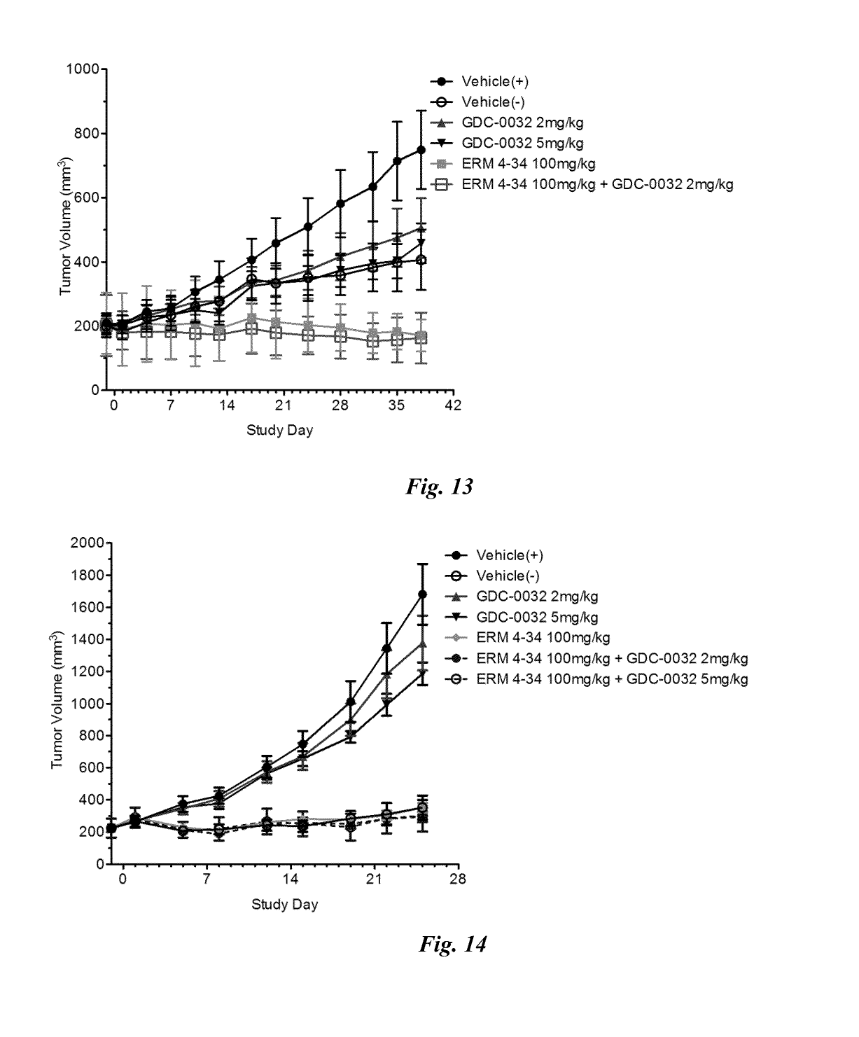

FIG. 13 shows the fitted tumor volume change over 38 days in cohorts of 8 immunocompromised mice bearing HCI-005 breast tumor (BC PDX model) xenografts harboring ESR1 L536P mutant, HER2+, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 4-34 from Table 3 at 100 mg/kg, and the combination of ERM 4-34 and GDC-0032. The HCI-005 breast tumor model is ESR1 mutant and HER2+. The combination of ERM 4-34 and GDC-0032 is not more efficacious than ERM 4-34 single agent anti-tumor activity in HCI-005.

FIG. 14 shows the fitted tumor volume change over 25 days in cohorts of 8 immunocompromised mice bearing HCI-011 breast tumor (BC PDX model) xenografts harboring ESR1 WT (wild type) and PIK3CA E545K (PI3Kα) mutation, dosed daily by 100 microliter (μl) PO (oral) administration with Vehicle (+), Vehicle (−), PI3K inhibitor GDC-0032 at 2 mg/kg and 5 mg/kg, estrogen receptor modulator (ERM) 4-34 from Table 3 at 100 mg/kg, and the combination of ERM 4-34 and GDC-0032. The HCI-011 breast tumor model is PI3K mutant E545K. The combination of ERM 4-34 and GDC-0032 is not more efficacious than ERM 4-34 single agent anti-tumor activity in HCI-011.

FIG. 15 shows the fitted tumor volume change over 26 days in cohorts of 8 or 9 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, PI3K inhibitor GDC-0032 at 2 mg/kg and 5 mg/kg, estrogen receptor modulator (ERM) 4-34 from Table 3 at 10 mg/kg and 100 mg/kg, and the combination of ERM 4-34 (10 mg/kg) and GDC-0032 (2 mg/kg). The combination of ERM 4-34 and GDC-0032 is not more efficacious than ERM 4-34 single agent anti-tumor activity in TamR1.

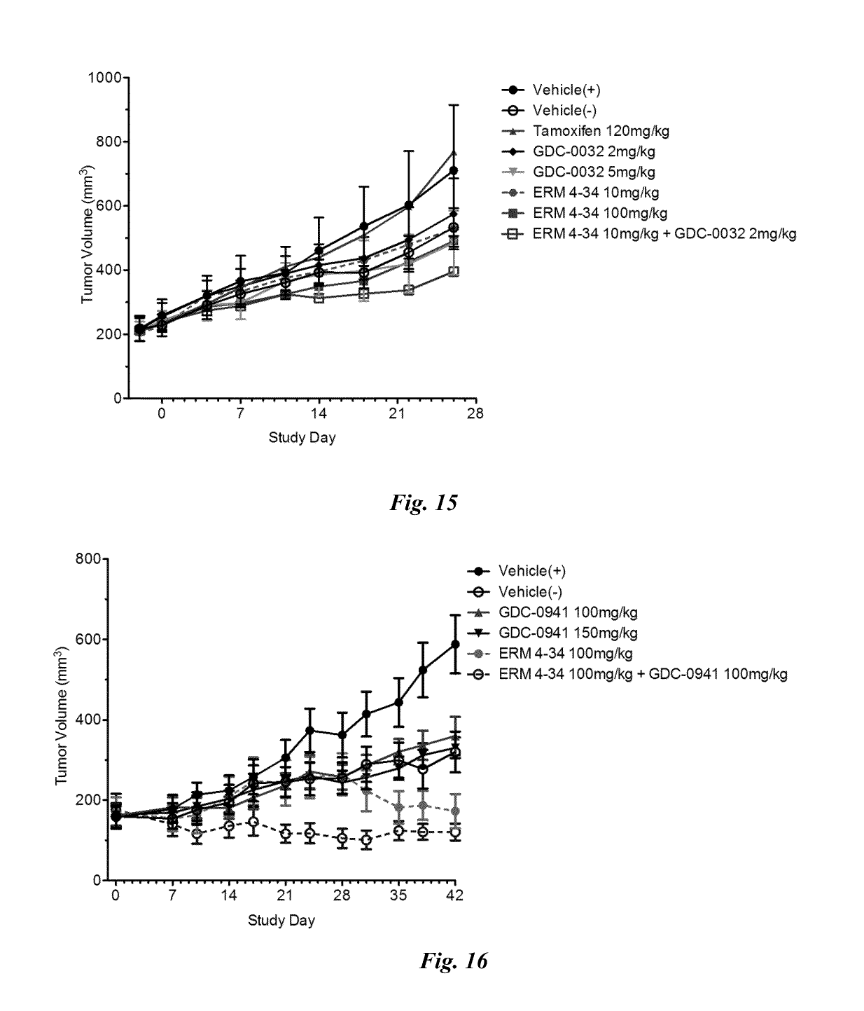

FIG. 16 shows the fitted tumor volume change over 42 days in cohorts of 7 immunocompromised mice bearing HCI-005 breast tumor (BC PDX model) xenografts harboring ESR1 L536P mutant, and HER2+, dosed daily by 100 microliter (μl) PO (oral) administration with Vehicle (+), Vehicle (−), pan-PI3K inhibitor GDC-0941 at 100 and 150 mg/kg, estrogen receptor modulator (ERM) 4-34 from Table 3 at 100 mg/kg, and the combination of ERM 4-34 (100 mg/kg) and GDC-0941 (100 mg/kg). The HCI-005 breast tumor model is ESR1 mutant and HER2+. ERM 4-34 enhances the efficacy of 100 mg/kg GDC-0941 by increasing tumor regressions in HCI-005.

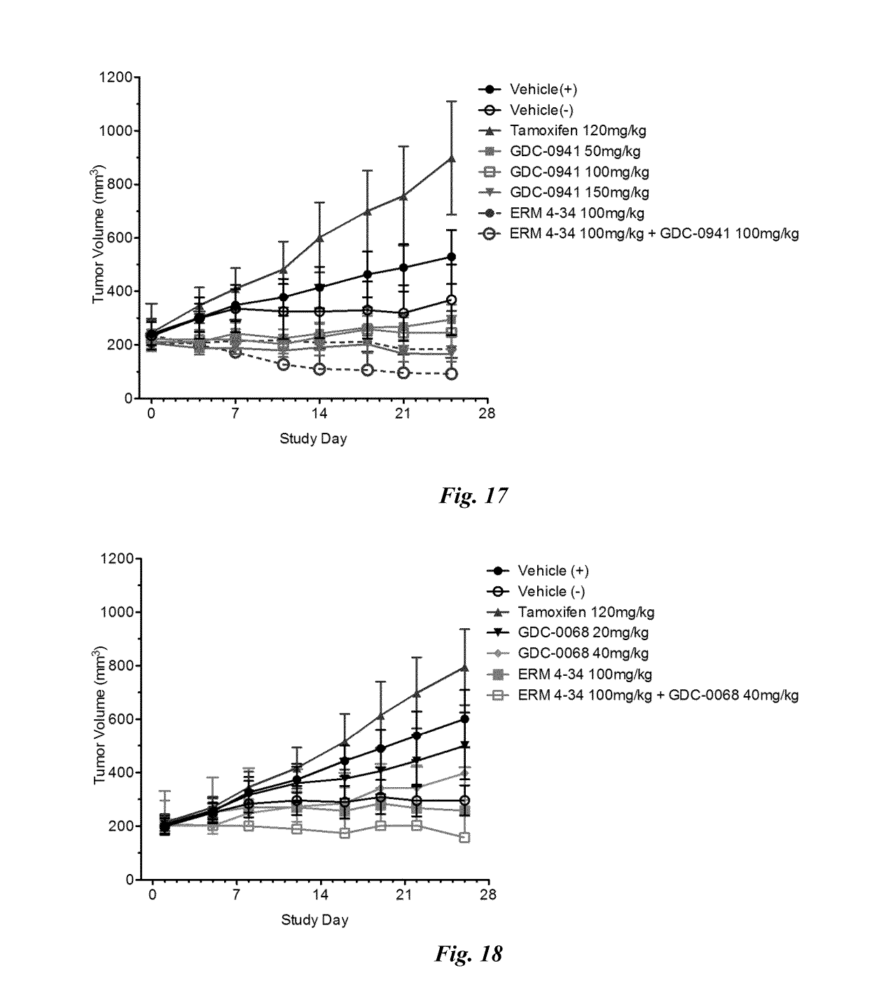

FIG. 17 shows the fitted tumor volume change over 25 days in cohorts of 8 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, pan-PI3K inhibitor GDC-0941 at 50, 100 and 150 mg/kg, estrogen receptor modulator (ERM) 4-34 from Table 3 at 100 mg/kg, and the combination of ERM 1-3 (100 mg/kg) and GDC-0941 (100 mg/kg). ERM 4-34 enhances the efficacy of 100 mg/kg GDC-0032 by increasing tumor regressions in TamR1.

FIG. 18 shows the fitted tumor volume change over 26 days in cohorts of 9 immunocompromised mice bearing TamR1 breast tumor model xenograft harboring PIK3CA E545K (PI3Kα) mutation and tamoxifen resistance, dosed daily by 100 microliter (μl) PO (oral) administration with Vehicle (+), Vehicle (−), tamoxifen citrate, AKT inhibitor GDC-0068 at 20 and 40 mg/kg, estrogen receptor modulator (ERM) 4-34 from Table 3 at 100 mg/kg, and the combination of ERM 4-34 (100 mg/kg) and GDC-0068 (40 mg/kg).

### EXAMPLES

These examples are provided for illustrative purposes only and not to limit the scope of the claims provided herein.

### Example 1

### Breast Cancer Clinical Trial

A non-limiting example of a breast cancer clinical trial in humans involving the use of a compound of Formula (A), (B), or (C), or a pharmaceutically acceptable salt thereof, is described below.

Purpose: The purposes of this study are to assess the efficacy of a compound of Formula (A), (B), or (C), or a pharmaceutically acceptable salt thereof, as single agent or in combination, as first- or second-line treatment of estrogen receptor (ER) positive metastatic breast cancer, collect information on any side effects the compound may cause, and evaluate the pharmacokinetic properties of the compound.

Intervention: Patients are administered 0.1-50 mg/kg of a compound of Formula (A), (B), or (C), or a pharmaceutically acceptable salt thereof, per day or twice a day as a single agent or in combination.

Outcome Measures: Primary Outcome Measures: tumor response and/or disease control.

Secondary Outcome Measures: (a) side-effects; (b) pharmacokinetic properties; (c) proportion of patients that have complete or partial response or stable disease at defined time points; (d) time to progression and overall survival; and (e) biomarkers predictive of clinical response.

Detailed Description: Patients will be given a compound of Formula (A), (B), or (C), or a pharmaceutically acceptable salt thereof, orally once or twice a day, alone or in combination. Prior to each dosing cycle, a physical exam, blood work and assessment of any side effects will be performed. Every 12 weeks the patient's cancer will be re-evaluated with either a CT scan or MRI to determine whether the treatment is working Participation in this study will last until disease progression or unacceptable toxicity.

Eligibility: Female subjects that are 18 years and older.

Inclusion Criteria: Histologically or cytologically confirmed diagnosis of invasive breast cancer, stage IV disease; at least one measurable target lesion as defined by RECIST that has not been previously treated with local therapy; post-menopausal status; ER positive breast cancer; HER2-negative breast cancer; up to one prior hormonal therapy for advanced or metastatic disease; ECOG performance status 0-1; life expectancy>12 weeks; adequate liver and bone marrow function: AST<2.5×ULN; Bilirubin<1.5×ULN; ANC>1,500/ul; platelet count>100,000/ul; normal PT and PTT; at least 2 weeks since prior radiation and recovered from treatment-related toxicity.

Exclusion Criteria: HER2-positive breast cancer; prior chemotherapy regimen for metastatic disease; history of, or presence of brain metastases; concurrent investigational drug treatment; prior bone marrow or stem cell transplant; history of other malignancy within the last 5 years, not including curatively-treated carcinoma in situ of the cervix or non-melanoma skin cancer; uncontrolled infection; active bleeding, or history of bleeding requiring transfusion; active cardiac disease; serious medical or psychiatric illness.

### Example 2

### Endometrial Carcinoma Clinical Trial

A non-limiting example of an endometrial carcinoma clinical trial in humans involving the use of a compound of Formula (A), (B), or (C), or a pharmaceutically acceptable salt thereof, is described below.

Purpose: The purposes of this study are to assess the efficacy of a compound of Formula (A), (B), or (C), or a pharmaceutically acceptable salt thereof, alone or in combination, in the treatment of advanced or metastatic endometrial carcinoma, collect information on any side effects the compound may cause, and evaluate the pharmacokinetic properties of the compound.

Intervention: Patients are administered 0.1-50 mg/kg of a compound of Formula (A), (B), or (C), or a pharmaceutically acceptable salt thereof, per day or twice a day, as a single agent or in combination.

Outcome Measures: Primary Outcome Measures: tumor response and/or disease control

Secondary Outcome Measures: (a) side-effects; (b) pharmacokinetic properties; (c) proportion of patients that have complete or partial response or stable disease at defined time points; (d) time to progression and overall survival; and (e) biomarkers predictive of clinical response.

Detailed Description: Patients will be given a compound of Formula (A), (B), or (C), or a pharmaceutically acceptable salt thereof, orally once or twice a day. Prior to each dosing cycle, a physical exam, blood work and assessment of any side effects will be performed. Every 12 weeks the patient's cancer will be re-evaluated with either a CT scan or MRI to determine whether the treatment is working Participation in this study will last until disease progression or unacceptable toxicity.

Eligibility: Female subjects that are 18 years and older.

Inclusion Criteria: Histologically or cytologically confirmed diagnosis of advanced or metastatic endometrial carcinoma; at least one measurable target lesion as defined by RECIST that has not been previously treated with local therapy; hormone receptor positive endometrial carcinoma; ECOG performance status 0-1; life expectancy>12 weeks; adequate liver and bone marrow function: AST<2.5×ULN; Bilirubin<1.5×ULN; ANC>1,500/ul; platelet count>100,000/ul; normal PT and PTT; at least 2 weeks since prior radiation and recovered from prior surgery or treatment-related toxicity.

Exclusion Criteria: History of, or presence of brain metastases; concurrent investigational drug treatment; prior bone marrow or stem cell transplant; history of other malignancy within the last 5 years, not including curatively-treated carcinoma in situ of the cervix or non-melanoma skin cancer; uncontrolled infection; active bleeding, or history of bleeding requiring transfusion; active cardiac disease; serious medical or psychiatric illness.

### Example 3

### Ovarian Cancer Clinical Trial

A non-limiting example of a ovarian cancer clinical trial in humans involving the use of a compound of Formula (A), (B), or (C), or a pharmaceutically acceptable salt thereof, is described below.

Purpose: The purposes of this study are to assess the efficacy of a compound of Formula (A), (B), or (C), or a pharmaceutically acceptable salt thereof, as a single agent or in combination, in the treatment of advanced ovarian cancer, collect information on any side effects the compound may cause, and evaluate the pharmacokinetic properties of the compound.

Intervention: Patients are administered 0.1-50 mg/kg of a compound of Formula (A), (B), or (C), or a pharmaceutically acceptable salt thereof, per day or twice a day, as a single agent or in combination.

Outcome Measures: Primary Outcome Measures: tumor response and/or disease control

Secondary Outcome Measures: (a) side-effects; (b) pharmacokinetic properties; (c) proportion of patients that have complete or partial response or stable disease at defined time points; (d) time to progression and overall survival; and (e) biomarkers predictive of clinical response.

Detailed Description: Patients will be given a compound of Formula (A), (B), or (C), or a pharmaceutically acceptable salt thereof, orally once or twice a day, as a single agent or in combination. Prior to each dosing cycle, a physical exam, blood work (including tumor markers, e.g., CA-125) and assessment of any side effects will be performed. Every 12 weeks the patient's cancer will be re-evaluated with either a CT scan or MRI to determine whether the treatment is working Participation in this study will last until disease progression or unacceptable toxicity.

Eligibility: Female subjects that are 18 years and older.

Inclusion Criteria: Histologically or cytologically confirmed diagnosis of advanced ovarian cancer; at least one measurable target lesion as defined by RECIST that has not been previously treated with local therapy; ER positive ovarian cancer; ECOG performance status 0-1; life expectancy>12 weeks; adequate liver and bone marrow function: AST<2.5×ULN; Bilirubin<1.5×ULN; ANC>1,500/ul; platelet count>100,000/ul; normal PT and PTT; at least 2 weeks since prior radiation and recovered from prior surgery or treatment-related toxicity.

Exclusion Criteria: History of, or presence of brain metastases; concurrent investigational drug treatment; prior bone marrow or stem cell transplant; history of other malignancy within the last 5 years, not including curatively-treated carcinoma in situ of the cervix or non-melanoma skin cancer; uncontrolled infection; active bleeding, or history of bleeding requiring transfusion; active cardiac disease; serious medical or psychiatric illness.

### Example 4

### ER-Positive NSCLC Clinical Trial

A non-limiting example of a ER-Positive NSCLC clinical trial in humans involving the use of a compound of Formula (A), (B), or (C), or a pharmaceutically acceptable salt thereof, is described below.

Purpose: The purposes of this study are to assess the efficacy of a compound of Formula (A), (B), or (C), or a pharmaceutically acceptable salt thereof, as single agent or in combination in the treatment of advanced or metastatic estrogen receptor (ER) positive non-small cell lung cancer (NSCLC), collect information on any side effects the compound may cause as single agent or in combination, and evaluate the pharmacokinetic properties of the compound as single agent or in combination.

Intervention: Patients are administered 0.1-50 mg/kg of a compound of Formula (A), (B), or (C), or a pharmaceutically acceptable salt thereof, per day or twice a day as single agent or in combination.

Outcome Measures: Primary Outcome Measures: tumor response and/or disease control.

Secondary Outcome Measures: (a) side-effects; (b) pharmacokinetic properties; (c) proportion of patients that have complete or partial response or stable disease at defined time points; (d) time to progression and overall survival; and (e) biomarkers predictive of clinical response.

Detailed Description: Patients will be given a compound of Formula (A), (B), or (C), or a pharmaceutically acceptable salt thereof, orally once or twice a day as single agent or in combination. Prior to each dosing cycle, a physical exam, blood work and assessment of any side effects will be performed. Every 12 weeks the patient's cancer will be re-evaluated with either a CT scan or MRI to determine whether the treatment is working. Participation in this study will last until disease progression or unacceptable toxicity.

Eligibility: Male and female subjects that are 18 years and older.

Inclusion Criteria: Histologically or cytologically confirmed diagnosis of advanced or metastatic ER-positive NSCLC; at least one measurable target lesion as defined by RECIST that has not been previously treated with local therapy; ECOG performance status 0-1; life expectancy>12 weeks; adequate liver and bone marrow function: AST<2.5×ULN; Bilirubin<1.5×ULN; ANC>1,500/ul; platelet count>100,000/ul; normal PT and PTT; at least 2 weeks since prior radiation and recovered from prior surgery or treatment-related toxicity.

Exclusion Criteria: History of, or presence of brain metastases; concurrent investigational drug treatment; prior bone marrow or stem cell transplant; history of other malignancy within the last 5 years, not including curatively-treated carcinoma in situ of the cervix or non-melanoma skin cancer; uncontrolled infection; active bleeding, or history of bleeding requiring transfusion; active cardiac disease; serious medical or psychiatric illness.

### Example 5

### Endometriosis Clinical Trial

A non-limiting example of an endometriosis clinical trial in humans involving the use of a compound of Formula (A), (B), or (C), or a pharmaceutically acceptable salt thereof, is described below.

Purpose: The purposes of this study are to assess the efficacy of a compound of Formula (A), (B), or (C), or a pharmaceutically acceptable salt thereof, as single agent or in combination in the treatment of patients with symptomatic/severe endometriosis, collect information on any side effects the compound may cause as single agent or in combination, and evaluate the pharmacokinetic properties of the compound as single agent or in combination.

Intervention: Patients are administered 0.1-50 mg/kg of a compound of Formula (A), (B), or (C), or a pharmaceutically acceptable salt thereof, per day or twice a day as single agent or in combination.

Outcome Measures: The outcome measures of this study are symptoms improvement and/or pain relief and shrinkage of endometrial tissue.

Detailed Description: Patients will be given a compound of Formula (A), (B), or (C), or a pharmaceutically acceptable salt thereof, orally once or twice a day as single agent or in combination. Prior to each dosing cycle, a physical exam, blood work and assessment of any side effects will be performed.

Eligibility: Female subjects that are 18 years and older.

Inclusion Criteria: Diagnosis of symptomatic endometriosis; pre- or peri-menopausal status; ECOG performance status 0-1; adequate liver and bone marrow function: AST<2.5×ULN; Bilirubin<1.5×ULN; ANC>1,500/ul; platelet count>100,000/ul; normal PT and PTT; at least 2 weeks since prior surgery or treatment-related toxicity.

Exclusion Criteria: Pregnancy or lactating; history of other malignancy within the last 5 years, not including curatively-treated carcinoma in situ of the cervix or non-melanoma skin cancer; concurrent investigational drug treatment; uncontrolled infection; active cardiac disease; aerious medical or psychiatric illness.

### Example 6

### Uterine Leiomyoma Clinical Trial

A non-limiting example of an uterine leiomyoma clinical trial in humans involving the use of a compound of Formula (A), (B), or (C), or a pharmaceutically acceptable salt thereof, is described below.

Purpose: The purposes of this study are to assess the efficacy of a compound of Formula (A), (B), or (C), or a pharmaceutically acceptable salt thereof, as single agent or in combination in the treatment of patients with symptomatic uterine leiomyoma, collect information on any side effects the compound may cause as single agent or in combination, and evaluate the pharmacokinetic properties of the compound as single agent or in combination.

Intervention: Patients are administered 0.1-50 mg/kg of a compound of Formula (A), (B), or (C), or a pharmaceutically acceptable salt thereof, per day or twice a day as single agent or in combination.

Outcome Measures: The outcome measures of this study are symptoms improvement and/or pain relief and shrinkage of leiomyomas.

Detailed Description: Patients will be given a compound of Formula (A), (B), or (C), or a pharmaceutically acceptable salt thereof, orally once or twice a day as single agent or in combination. Prior to each dosing cycle, a physical exam, blood work and assessment of any side effects will be performed.

Eligibility: Female subjects that are 18 years and older.

Inclusion Criteria: Diagnosis of symptomatic uterine leiomyoma; pre- or peri-menopausal status; ECOG performance status 0-1; adequate liver and bone marrow function: AST<2.5×ULN; Bilirubin<1.5×ULN; ANC>1,500/ul; platelet count>100,000/ul; normal PT and PTT; at least 2 weeks since prior surgery or treatment-related toxicity.

Exclusion Criteria: Pregnancy or lactating; history of other malignancy within the last 5 years, not including curatively-treated carcinoma in situ of the cervix or non-melanoma skin cancer; concurrent investigational drug treatment; uncontrolled infection; active cardiac disease; serious medical or psychiatric illness.

### Example 7

### In Vitro Cell Proliferation Assay

Efficacy of estrogen receptor modulator compounds and chemotherapeutic compounds are measured by a cell proliferation assay employing the following protocol (Mendoza et al (2002) Cancer Res. 62:5485-5488).

The CellTiter-Glo® Luminescent Cell Viability Assay is a homogeneous method to determine the number of viable cells in culture based on quantitation of the ATP present, which signals the presence of metabolically active cells. The CellTiter-Glo® Assay is designed for use with multiwell plate formats, making it ideal for automated high-throughput screening (HTS), cell proliferation and cytotoxicity assays. The homogeneous assay procedure involves adding a single reagent (CellTiter-Glo® Reagent) directly to cells cultured in serum-supplemented medium. Cell washing, removal of medium or multiple pipetting steps are not required. The Cell Titer-Gb® Luminescent Cell Viability Assay, including reagents and protocol are commercially available (Promega Corp., Madison, Wis., Technical Bulletin TB288).

The assay assesses the ability of compounds to enter cells and inhibit cell proliferation. The assay principle is based on the determination of the number of viable cells present by quantitating the ATP present in a homogenous assay where addition of the Cell Titer-Gloms reagent results in cell lysis and generation of a luminescent signal through the luciferase reaction. The luminescent signal is proportional to the amount of ATP present.

Procedure: Day 1—Seed Cell Plates (384-well black, clear bottom, microclear, TC plates with lid from Falcon #353962), Harvest cells, Seed cells at 1000 cells per 54 μl per well into 384 well Cell Plates for 3 days assay. Cell Culture Medium: RPMI or DMEM high glucose, 10% Fetal Bovine Serum, 2 mM L-Glutamine, P/S. Incubate O/N (overnight) at 37° C., 5% CO₂.

Day 2—Add Drug to Cells, Compound Dilution, DMSO Plates (serial 1:2 for 9 points). Add 20 μl of compound at 10 mM in the 2nd column of 96 well plate. Perform serial 1:2 across the plate (10 μl+20 μl 100% DMSO) for a total of 9 points using Precision Media Plates 96-well conical bottom polypropylene plates from Nunc (cat.#249946) (1:50 dilution). Add 147 μl of Media into all wells. Transfer 3 μl of DMSO+ compound from each well in the DMSO Plate to each corresponding well on Media Plate using Rapidplate® (Caliper, a Perkin-Elmer Co.). For 2 drug combination studies, transfer one drug 1.5 μl of DMSO+compound from each well in the DMSO Plate to each corresponding well on Media Plate using Rapidplate. Then, transfer another drug 1.5 μl to the medium plate.

Drug Addition to Cells, Cell Plate (1:10 dilution): Add 6 μl of media+compound directly to cells (54 μl of media on the cells already). Incubate 3 days at 37° C., 5% CO₂ in an incubator that will not be opened often.

Day 5—Develop Plates, Thaw Cell Titer Glo Buffer at room temperature: Remove Cell Plates from 37° C. and equilibrate to room temperature for about 30 minutes. Add Cell Titer-Glo® Buffer to Cell Titer-Glo® Substrate (bottle to bottle). Add 30 μl Cell Titer-Glo® Reagent (Promega cat.# G7572) to each well of cells. Place on plate shaker for about 30 minutes. Read luminescence on Analyst HT Plate Reader (half second per well).

Cell viability assays and combination assays: Cells were seeded at 1000-2000 cells/well in 384-well plates for 16 h. On day two, nine serial 1:2 compound dilutions were made in DMSO in a 96 well plate. The compounds were further diluted into growth media using a Rapidplate® robot (Zymark Corp., Hopkinton, Mass.). The diluted compounds were then added to quadruplicate wells in 384-well cell plates and incubated at 37° C. and 5% CO₂. After 4 days, relative numbers of viable cells were measured by luminescence using Cell Titer-Glo® (Promega) according to the manufacturer's instructions and read on a Wallac Multilabel Reader® (PerkinElmer, Foster City). EC50 values were calculated using Prism® 4.0 software (GraphPad, San Diego). Drugs in combination assays were dosed starting at 4× EC₅₀ concentrations. If cases where the EC50 of the drug was >2.5 μM, the highest concentration used was 10 μM. Estrogen receptor modulator compounds and chemotherapeutic agents were added simultaneously or separated by 4 hours (one before the other) in all assays.

An additional exemplary in vitro cell proliferation assay includes the following steps:

1. An aliquot of 100 μl of cell culture containing about 10⁴ cells (see Table 3 for cell lines and tumor type) in medium was deposited in each well of a 384-well, opaque-walled plate.

2. Control wells were prepared containing medium and without cells.

3. The compound was added to the experimental wells and incubated for 3-5 days.

4. The plates were equilibrated to room temperature for approximately 30 minutes.

5. A volume of CellTiter-Glo® Reagent equal to the volume of cell culture medium present in each well was added.

6. The contents were mixed for 2 minutes on an orbital shaker to induce cell lysis.

7. The plate was incubated at room temperature for 10 minutes to stabilize the luminescence signal.

8. Luminescence was recorded and reported in graphs as RLU=relative luminescence units.

9. Analyze using the Chou and Talalay combination method and Dose-Effect Analysis with CalcuSyn® software (Biosoft, Cambridge, UK) in order to obtain a Combination Index.

Alternatively, cells were seeded at optimal density in a 96 well plate and incubated for 4 days in the presence of test compound. Alamar Blue™ was subsequently added to the assay medium, and cells were incubated for 6 h before reading at 544 nm excitation, 590 nm emission. EC₅₀ values were calculated using a sigmoidal dose response curve fit.

Alternatively, Proliferation/Viability was analyzed after 48 hr of drug treatment using Cell Titer-Glo® reagent (Promega Inc., Madison, Wis.). DMSO treatment was used as control in all viability assays. IC₅₀ values were calculated using XL fit software (IDBS, Alameda, Calif.)

The cell lines were obtained from either ATCC (American Type Culture Collection, Manassas, Va.) or DSMZ (Deutsche Sammlung von Mikroorganismen and Zellkulturen GmbH, Braunschweig, DE). Cells were cultured in RPMI 1640 medium supplemented with 10% fetal bovine serum, 100 units/ml penicillin, 2 mM L-glutamine, and 100 mg/ml streptomycin (Life Technology, Grand Island, N.Y.) at 37° C. under 5% CO₂.

### Example 8

### In Vivo Mouse Tumor Xenograft Efficacy

Mice: Female severe combined immunodeficiency mice (Fox Chase SCID®, C.B-17/IcrHsd, Harlan) or nude mice (Taconic Farms, Harlan) were 8 to 9 weeks old and had a BW range of 15.1 to 21.4 grams on Day 0 of the study. The animals were fed ad libitum water (reverse osmosis, 1 ppm Cl) and NIH 31 Modified and Irradiated Lab Diet® consisting of 18.0% crude protein, 5.0% crude fat, and 5.0% crude fiber. The mice were housed on irradiated ALPHA-Dri® bed-o'Cobs® Laboratory Animal Bedding in static microisolators on a 12-hour light cycle at 21-22° C. (70-72° F.) and 40-60% humidity. PRC specifically complies with the recommendations of the Guide for Care and Use of Laboratory Animals with respect to restraint, husbandry, surgical procedures, feed and fluid regulation, and veterinary care. The animal care and use program at PRC is accredited by the Association for Assessment and Accreditation of Laboratory Animal Care International (AAALAC), which assures compliance with accepted standards for the care and use of laboratory animals.

Tumor Implantation: Xenografts were initiated with cancer cells. Cells were cultured in RPMI 1640 medium supplemented with 10% fetal bovine serum, 2 mM glutamine, 100 units/mL penicillin, 100 μg/mL streptomycin sulfate and 25 μg/mL gentamicin. The cells were harvested during exponential growth and resuspended in phosphate buffered saline (PBS) at a concentration of 5×10⁶ or 10×10⁶ cells/mL depending on the doubling time of the cell line. Tumor cells were implanted subcutaneously in the right flank, and tumor growth was monitored as the average size approached the target range of 100 to 150 mm3. Twenty-one days after tumor implantation, designated as Day 0 of the study, the mice were placed into four groups each consisting of ten mice with individual tumor volumes ranging from 75-172 mm3 and group mean tumor volumes from 120-121 mm3 (see Appendix A). Volume was calculated using the formula:

Tumor Volume (mm³)=(w²×1)/2, where w=width and 1=length in mm of a tumor. Tumor weight may be estimated with the assumption that 1 mg is equivalent to 1 mm3 of tumor volume.

Therapeutic Agents: estrogen receptor modulator compounds and chemotherapeutic agents were typically prepared from dry powders, stored at room temperature, and protected from light. Drug doses were prepared weekly in 0.5% methylcellulose: 0.2% Tween 80 in deionized water (“Vehicle”) and stored at 4° C. Vehicle (+) is solvent/buffer with ethynyl estradiol (ethinyl estradiol, EE2) at 0.1 mg/kg. Vehicle (−) is solvent/buffer without ethynyl estradiol. Doses of compounds were prepared on each day of dosing by diluting an aliquot of the stock with sterile saline (0.9% NaCl). All doses were formulated to deliver the stated mg/kg dosage in a volume of 0.2 mL per 20 grams of body weight (10 mL/kg).

Treatment: All doses were scaled to the body weights of the individual animals and were provided by the route indicated in each of the figures.

Endpoint: Tumor volume was measured in 2 dimensions (length and width), using Ultra Cal IV calipers (Model 54 10 111; Fred V. Fowler Company), as follows: tumor volume (mm³)=(length×width)×0.5 and analyzed using Excel version 11.2 (Microsoft Corporation). A linear mixed effect (LME) modeling approach was used to analyze the repeated measurement of tumor volumes from the same animals over time (Pinheiro J, et al. nlme: linear and nonlinear mixed effects models. R package version 3.1 92. 2009; Tan N, et al. Clin. Cancer Res. 2011; 17(6):1394-1404). This approach addresses both repeated measurements and modest dropouts due to any non-treatment-related death of animals before study end. Cubic regression splines were used to fit a nonlinear profile to the time courses of log 2 tumor volume at each dose level. These nonlinear profiles were then related to dose within the mixed model. Tumor growth inhibition as a percentage of vehicle control (% TGI) was calculated as the percentage of the area under the fitted curve (AUC) for the respective dose group per day in relation to the vehicle, using the following formula: % TGI=100×(1−AUC_{dose}/AUCᵥₑₕ). Using this formula, a TGI value of 100% indicates tumor stasis, a TGI value of >1% but <100% indicates tumor growth delay, and a TGI value of >100% indicates tumor regression. Partial response (PR) for an animal was defined as a tumor regression of >50% but <100% of the starting tumor volume. Complete response (CR) was defined as 100% tumor regression (i.e., no measurable tumor) on any day during the study.

Toxicity: Animals were weighed daily for the first five days of the study and twice weekly thereafter. Animal body weights were measured using an Adventurer Pro® AV812 scale (Ohaus Corporation). Percent weight change was calculated as follows: body weight change (%)=[(weight_{day new}−weight_{day 0})/weight_{day 0}]×100. The mice were observed frequently for overt signs of any adverse, treatment-related side effects, and clinical signs of toxicity were recorded when observed. Acceptable toxicity is defined as a group mean body weight (BW) loss of less than 20% during the study and not more than one treatment-related (TR) death among ten treated animals. Any dosing regimen that results in greater toxicity is considered above the maximum tolerated dose (MTD). A death is classified as TR if attributable to treatment side effects as evidenced by clinical signs and/or necropsy, or may also be classified as TR if due to unknown causes during the dosing period or within 10 days of the last dose. A death is classified as NTR if there is no evidence that death was related to treatment side effects.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method for treating an ER-related disease or condition in a patient comprising administering a therapeutic combination as a combined formulation or by alternation to the patient, wherein the therapeutic combination comprises a therapeutically effective amount of an estrogen receptor modulator compound selected from Formulas (A), (B), or (C), and a therapeutically effective amount of a second therapeutic agent,
wherein the compound of Formula (A) has the structure: 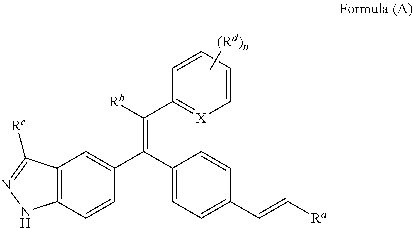
where,
R^{a} is —CO₂H or a 5-membered heterocycle selected from the group consisting of 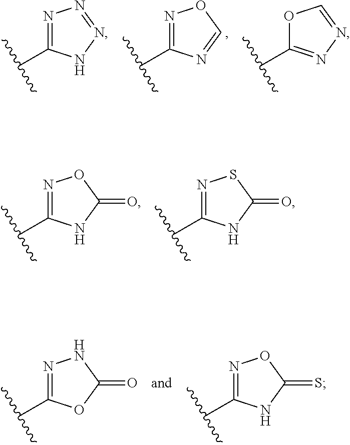
R^{b} is C₁-C₆ alkyl or C₃-C₆ cycloalkyl;
R^{c} is H or F;
each R^{d} is independently selected from H, halogen, —CN, —OR^{e}, —NHR^{e}, —NR^{e}R^{f}, —SR^{e}, —S(═O)R^{f}, —S(═O)₂R^{f}, C₁-C₆ alkyl, and C₁-C₆ fluoroalkyl;
each R^{e} is independently selected from H, —C(═O)R^{f}, —C(═O)OR^{f}, —C(═O)NHR^{f}, C₁-C₆ alkyl, C₁-C₆ fluoroalkyl, C₃-C₆ cycloalkyl, substituted or unsubstituted monocyclic C₂-C₆ heterocycloalkyl, substituted or unsubstituted phenyl, and substituted or unsubstituted monocyclic heteroaryl;
each R^{f} is independently selected from C₁-C₆ alkyl, C₁-C₆ fluoroalkyl, C₃-C₆ cycloalkyl, substituted or unsubstituted monocyclic C₂-C₆ heterocycloalkyl, substituted or unsubstituted phenyl, and substituted or unsubstituted monocyclic heteroaryl;
X is CH or N; and
n is 0, 1, or 2, or a pharmaceutically acceptable salt, or solvate thereof;
wherein the compound of Formula (B) has the structure: 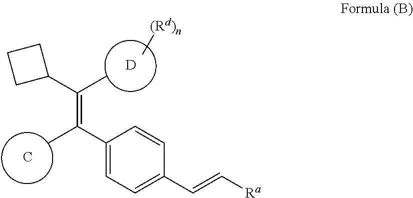
where,
R^{a} is —CO₂H or a 5-membered heterocycle selected from the group consisting of 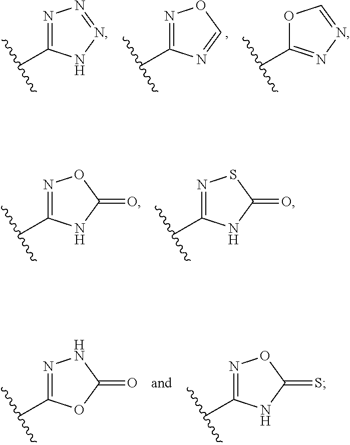
ring C is 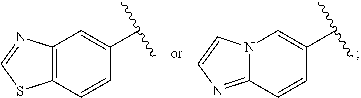 [SMILES: *c1ccc2scnc2c1 ; *c1ccc2nccn2c1]
ring D is phenyl or thienyl;
each R^{d} is independently selected from H, halogen, —CN, —OR^{e}, —NHR^{e}, —NR^{e}R^{f}, —SR^{e}, —S(═O)R^{f}, —S(═O)₂R^{f}, C₁-C₆ alkyl, and C₁-C₆ fluoroalkyl;
each R^{e} is independently selected from H, —C(═O)R^{f}, —C(═O)OR^{f}, —C(═O)NHR^{f}, C₁-C₆ alkyl, C₁-C₆ fluoroalkyl, C₃-C₆ cycloalkyl, substituted or unsubstituted monocyclic C₂-C₆ heterocycloalkyl, substituted or unsubstituted phenyl, and substituted or unsubstituted monocyclic heteroaryl;
each R^{f} is independently selected from C₁-C₆ alkyl, C₁-C₆ fluoroalkyl, C₃-C₆ cycloalkyl, substituted or unsubstituted monocyclic C₂-C₆ heterocycloalkyl, substituted or unsubstituted phenyl, and substituted or unsubstituted monocyclic heteroaryl;
n is 0, 1, or 2; or a pharmaceutically acceptable salt, or solvate thereof;
wherein the compound of Formula (C) has the structure: 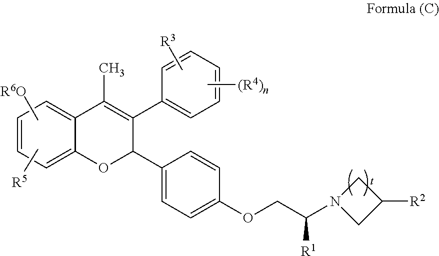
where,
R¹ is H, C₁-C₄ alkyl, or C₁-C₄ fluoroalkyl;
R² is H, F, C₁-C₄ alkyl or C₁-C₄ fluoroalkyl;
R³ is H, halogen, —CN, —OR⁶, —NHR⁶, —NR⁶R⁷, —SR⁶, —S(═O)R⁷, —S(═O)₂R⁷, C₁-C₄ alkyl, or C₁-C₄ fluoroalkyl;
each R⁴ is independently selected from H, halogen, —CN, —OH, C₁-C₆ alkyl, C₁-C₄ fluoroalkyl, C₁-C₄ fluoroalkoxy, and C₁-C₄ alkoxy;
each R⁵ is H, F, Cl, —OH, —CH₃, —CF₃, or —OCH₃;
each R⁶ is independently selected from H, —C(═O)R⁷, —C(═O)OR⁷, —C(═O)NHR⁷, C₁-C₆ alkyl, C₁-C₆ fluoroalkyl, C₃-C₆ cycloalkyl, substituted or unsubstituted C₂-C₆ heterocycloalkyl, substituted or unsubstituted phenyl, and substituted or unsubstituted monocyclic heteroaryl; each R⁷ is independently selected from C₁-C₆ alkyl, C₁-C₆ fluoroalkyl, substituted or unsubstituted C₃-C₆ cycloalkyl, substituted or unsubstituted monocyclic C₂-C₆ heterocycloalkyl, substituted or unsubstituted phenyl, and substituted or unsubstituted monocyclic heteroaryl;
n is 0, 1, or 2;
t is 1 or 2;
or a pharmaceutically acceptable salt, or solvate thereof;
wherein the second therapeutic agent is an aromatase inhibitor, a phosphoinositide 3-kinase (PI3K)/mTOR pathway inhibitor, a CDK 4/6 inhibitor, a HER-2 inhibitor, an EGFR inhibitor, a PD-1 inhibitor, poly ADP-ribose polymerase (PARP) inhibitor, a histone deacetylase (HDAC) inhibitor, an HSP90 inhibitor, a VEGFR inhibitor, an AKT inhibitor, chemotherapy, or any combination thereof.

2. The method of claim 1 wherein the therapeutically effective amounts of the estrogen receptor modulator compound and the second therapeutic agent are administered as a combined formulation.

3. The method of claim 1 wherein the therapeutically effective amounts of the estrogen receptor modulator compound and the second therapeutic agent are administered by alternation.

4. The method of claim 1 wherein the mammal is administered with the estrogen receptor modulator compound and subsequently administered with the second therapeutic agent.

5. The method of claim 1 wherein the therapeutic combination is administered by a dosing regimen where the therapeutically effective amount of the estrogen receptor modulator compound is administered in a range from twice daily to once every three weeks, and the therapeutically effective amount of the second therapeutic agent is administered in a range from twice daily to once every three weeks.

6. The method of claim 5 wherein the dosing regimen is repeated one or more times.

7. The method of claim 1 wherein the estrogen receptor modulator compound and the second therapeutic agent are each administered in an amount from about 1 mg to about 1000 mg per unit dosage form.

8. The method of claim 1 wherein the estrogen receptor modulator compound and the second therapeutic agent are administered in a ratio of about 1:50 to about 50:1 by weight.

9. The method of claim 1 wherein administration of the therapeutic combination results in a synergistic effect.

10. The method of claim 1 wherein the ER-related disease or condition is cancer.

11. The method of claim 10 wherein the cancer is selected from breast, cervical, colon, endometrial, glioma, lung, melanoma, ovarian, pancreatic, and prostate.

12. The method of claim 11 wherein the cancer expresses a PIK3CA mutant selected from E542K, E545K, Q546R, H1047L and H1047R, a K-ras mutant, or a PTEN mutant.

13. The method of claim 11 wherein the cancer is breast cancer.

14. The method of claim 13 wherein the breast cancer is metastatic, hormone resistant, estrogen receptor positive, estrogen receptor negative, progesterone receptor negative, HER2 positive, or HER2 negative breast cancer.

15. The method of claim 13 wherein the breast cancer is resistant to treatment with an aromatase inhibitor.

16. The method of claim 15 wherein the aromatase inhibitor is anastrozole, letrozole, or exemestane.

17. The method of claim 14 wherein the breast cancer is Basal or Luminal subtype.

18. The method of any of claim 1 wherein the patient is a pre-menopausal or post-menopausal female patient.

19. The method of claim 1 wherein the patient has failed one or more anti-cancer therapies.

20. The method of claim 1 wherein the second therapeutic agent is a phosphoinositide 3-kinase (PI3K)/mTOR pathway inhibitor.

21. The method claim 20 wherein the phosphoinositide 3-kinase (PI3K)/mTOR pathway inhibitor is GDC-0032 or GDC-0941.

22. The method of claim 20 wherein a biological sample obtained from the patient, prior to administration of the therapeutic combination to the patient, has been tested for PIK3CA or PTEN mutation status, and wherein PIK3CA or PTEN mutation status is indicative of therapeutic responsiveness by the patient to the therapeutic combination.

23. The method of claim 20 wherein a biological sample has been tested by measuring functional PI3K protein level after administration of the therapeutic combination, wherein a change in the level of functional PI3K protein indicates that the patient will be resistant or responsive to the therapeutic combination.

24. The method of claim 1 wherein the second therapeutic agent is an AKT inhibitor.

25. The method of claim 24 wherein the AKT inhibitor is GDC-0068.

26. The method of claim 1 wherein the estrogen receptor modulator is a compound of Formula (A-1) having the structure: 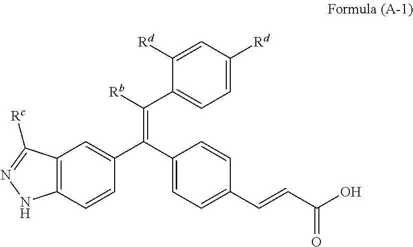
or a pharmaceutically acceptable salt, or solvate thereof.

27. The method of claim 26 wherein the estrogen receptor modulator is a Formula (A) compound selected from 1-1, 1-2, 1-3, 1-4, 1-5, 1-6, 1-7, 1-8, 1-9, 1-10, 1-11, or 1-12, or a pharmaceutically acceptable salt, or solvate thereof.

28. The method of claim 27 wherein the estrogen receptor modulator is 1-3.

29. The method of claim 1 wherein the estrogen receptor modulator is a Formula (B) compound selected from 2-1, 2-2, 2-3, 2-4, or 2-5, or a pharmaceutically acceptable salt, or solvate thereof.

30. The method of claim 1 wherein the estrogen receptor modulator of Formula (C) is selected from Formulas (C-1), (C-2), (C-3), (C-4), (C-5), and (C-6), having the structures: 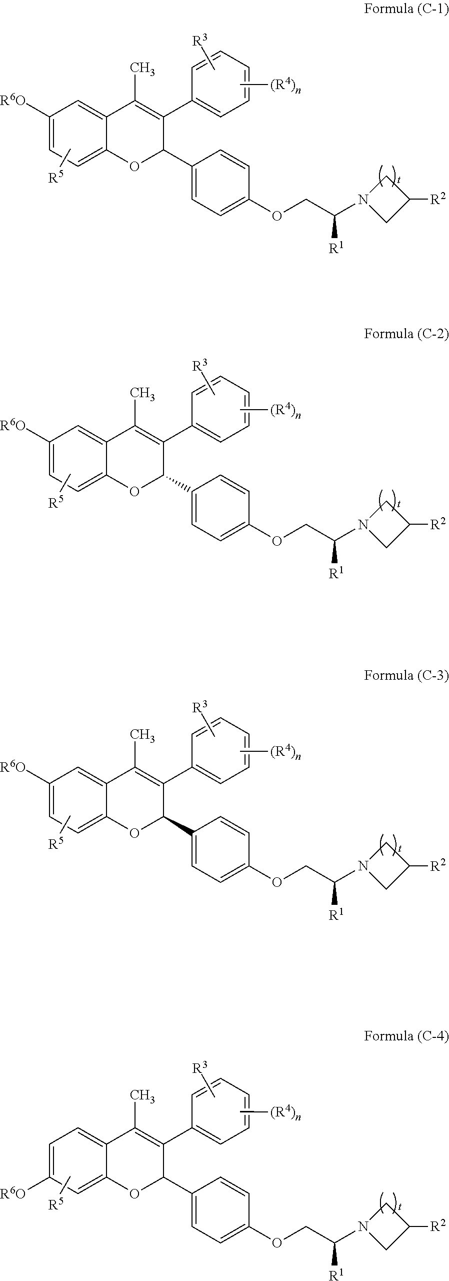 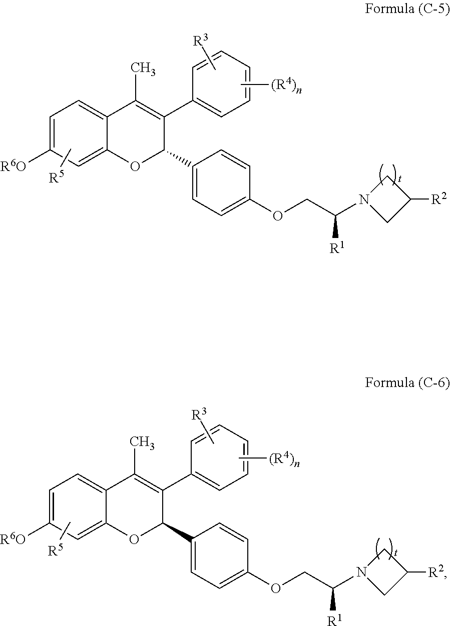
or a pharmaceutically acceptable salt, or solvate thereof.

31. The method of claim 30, wherein the estrogen receptor modulator is a Formula (C) compound selected from 4-1, 4-2, 4-3, 4-4, 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 4-25, 4-26, 4-27, 4-28, 4-29, 4-30, 4-31, 4-32, 4-33, 4-34, 4-35; or a pharmaceutically acceptable salt, or solvate thereof.

32. The method of claim 31, wherein the estrogen receptor modulator is 4-34.
